(19) United States
(12) Patent Application Publication
(10) Pub. No.: US 2024/0377385 A1
(43) Pub. Date: Nov. 14, 2024

(54) METHODS AND COMPOSITIONS OF MATTER FOR TREATING CANCER AND PREDICTING RESPONSE TO THERAPY

(71) Applicant: ICCA INC., Las Vegas, NV (US)

(72) Inventors: Thomas E. Ichim, San Diego, CA (US); Gregory DiRienzo, Dana Point, CA (US); Yi Ma, Dana Point, CA (US); Nicole DiRienzo, Newport Beach, CA (US)

(21) Appl. No.: 18/661,162

(22) Filed: May 10, 2024

Related U.S. Application Data
(60) Provisional application No. 63/501,833, filed on May 12, 2023.

(51) Int. Cl. G01N 33/50; C12N 5/09

(52) U.S. Cl. CPC G01N 33/5011; C12N 5/0693; C12N 2500/02; C12N 2500/12; C12N 2502/1323; C12N 2503/02; C12N 2510/00; G01N 2800/52

(57) ABSTRACT

Disclosed herein are methods and compositions for treating cancer. Further disclosed herein are methods and compositions useful for predicting the response of a patient bearing a tumor to therapy of such tumor.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims priority to U.S. Provisional Patent Application No. 63/501,833, filed May 12, 2023, which is hereby incorporated by reference in its entirety.

### BACKGROUND

### Field

The present disclosure relates to cancer treatment and prediction of cancer response to therapeutic agents.

### Description

Chemotherapy and other types of cancer therapy have been characterized by excellent efficacy in some patients and poor responsiveness in others. In many cases this is due to heterogeneity of tumors. Some tumors possess sensitivity to one type of therapy, whereas others to other types, even though many times tumors histologically and even molecularly look identical.

Previous attempts to define in vitro sensitivity of cancers to various therapies included in vitro assays in which tumor cells were extracted, expanded ex vivo, and assayed against a variety of chemotherapeutic agents. These tests have provided some degree of in vivo predictive activity, however they still possessed a degree of unreliability.

It is known that tumor cells that are extracted possess primarily differentiated cancer cells and not tumor stem cells. Tumor stem cells comprise a minority of biopsy samples and also are not expanded properly ex vivo under standard culture conditions.

Since it is the tumor stem cells that cause the cancer to come back, it is important to identify chemotherapeutic and other agents that have potential to induce cure as opposed to temporary remission.

Although it seems like an overly profound statement, it is believed that cancer stem cells (CSCs) are the origins of cancer. The reason is that these cells have the ability to self-renew and differentiate to form a tumor hierarchy. Furthermore, CSCs can migrate and be resistant to anti-cancer drug therapy. Since CSCs are believed to be a rare subset in tumors, they have been characterized based on cell surface markers and tumor-initiating activity in xenograft transplantations. Particular studies have shown that stem cell markers are capable of predicting cancer stem cells. For example, markers such as CD133, CD44, EpCAM, CD 166, ALDH and CD26 have been demonstrated to possess ability to be useful in isolation of cancer stem cells.

### SUMMARY

Disclosed are means, treatments, and compositions of matter useful for reducing and/or eradicating one or more neoplastic masses while inducing systemic immunological memory to prevent relapse. In one embodiment the present disclosure provides enhancement of immunity, modulation/repair of microbiome, manipulation of the cancer microenvironment, augmentation of tumor immunogenicity, tumor reductive procedures, enhancement of immunogenicity of tumor reductive procedures, and induction of antigen-specific immunological memory.

The present disclosure also relates to methods and compositions of matter useful for predicting the response of a patient bearing a tumor to therapy of such tumor. In one embodiment tumor tissue is collected either through biopsy, or circulating tumor cells, or tumor cell released extracellular vesicles. In one embodiment tumor cells are grown in media conditioned by regenerative cells in order to provide a more accurate representation of in vivo tumor conditions which is not represented by existing means in the art. In contrast to current means of predicting tumor responses, the present disclosure provides means of assessing response of tumor stem cells to therapy. This provides more accuracy in predicting the in vivo response to cancer therapy with higher efficiency.

Disclosed herein are methods of treating cancer. In some embodiments, the method includes the steps of: a) assessing a cancer patient for sensitivity of the cancer to a variety of therapeutic approaches; b) reducing tumor hypoxia and/or tumor acidosis in the cancer; c) altering a tumor microenvironment of the cancer; and d) administering said cancer therapeutic approaches.

In some embodiments, assessing a cancer patient for sensitivity to therapeutic approaches comprises assessing cancer samples ex vivo for sensitivity to chemotherapy.

In some embodiments, reducing tumor hypoxia and/or tumor acidosis comprises administering sodium bicarbonate, ozone therapy or hydrogen peroxide.

In some embodiments, altering the tumor microenvironment comprises inducing localized hyperthermia, administering localized high intensity ultrasound, administering localized radiation therapy, localized administration of an immunogenic substance, localized administration of a vaccine adjuvant, localized administration of a vaccine adjuvant, administering low dose chemotherapy, or administering immunogenic chemotherapy. In some embodiments, the vaccine adjuvant is selected from the group consisting of: Poly IC, incomplete Freunds Adjuvant, and BCG.

In some embodiments, the method further includes administering an antigen-specific immune stimulatory means or an antigen-nonspecific stimulatory means. In some embodiments, the antigen-specific immune stimulatory means comprises autologous tumor immunization. In some embodiments, said autologous tumor immunization comprises administration of tumor lysate, tumor exosome, tumor lysate pulsed dendritic cells, tumor exosomes pulsed dendritic cells, or allogeneic tumor cell lines. In some embodiments, the allogeneic tumor cell lines are made immunogenic by transfection with one or more immunogenic genes. In some embodiments, said immunogenic genes are selected from the group consisting of: CD40, CD80, CD86, and GM-CSF.

In some embodiments, assessing sensitivity to chemotherapy comprises assessing cancer cell death. In some embodiments, cancer cell death is assessed by mitochondrial depolarization or by increased membrane permeability to molecules which normally reside outside of said membrane. In some embodiments, said cancer cell death comprises apoptosis, necrosis, or immunogenic cell death. In some embodiments, said immunogenic cell death comprises ferroptosis, pyroptosis, or necroptosis.

In some embodiments, assessing the cancer patient for sensitivity to therapeutic approaches comprises assessing cancer samples ex vivo for sensitivity to immunotherapy, metabolic therapy, nucleic acid therapy, electromagnetic therapy, radiation therapy, cryotherapy, hyperthermia, or treatment.

Further disclosed herein are methods of predicting a cancer patient's response to therapy. In some embodiments, the method includes the steps of: a) obtaining a tumor sample from said cancer patient; b) exposing said tumor samples to a matrix replicating conditions found inside said tumor microenvironment; c) exposing said tumor sample in said conditions replicating said cancer microenvironment to various cancer treatments being contemplated for us in said cancer patients; d) observing effects of said treatments on said cancer cells; and e) recommending to caregiver of said patient which treatments have evoked an in vitro response.

In some embodiments, the tumor sample is obtained as circulating tumor cells from peripheral blood. In some embodiments, the circulating tumor cells are selected based on expression of PECAM, CD133, CD34, stem cell factor receptor, c-met, TGF-beta receptor, interleukin-1 receptor, interleukin-17 receptor, mucin-1, LIF receptor, IL-10 receptor, based on ability to export rhodamine 231, or based on enhanced binding to lectins.

In some embodiments, the tumor sample comprises a tumor-derived microvesicle. In some embodiments, the tumor derived microvesicles are concentrated and exposed to a fibroblast or fibroblast like-population. In some embodiments, the fibroblast or fibroblast like-population is transfected with one or more oncogenes.

In some embodiments, the tumor sample comprises cancer cells, and the method comprises growing cancer cells under conditions of stem cell supernatant.

### BRIEF DESCRIPTION OF THE DRAWINGS

Features of examples of the present disclosure will become apparent by reference to the following detailed description and drawings. In addition to the features described herein, additional features and variations will be readily apparent from the following descriptions of the drawings and exemplary embodiments. It is to be understood that these drawings depict typical embodiments, and are not intended to be limiting in scope.

FIG. 1 is a bar graph illustrating results from treating mice with a first exemplary combination immunotherapy protocol.

FIG. 2 is a bar graph illustrating results from treating mice with a second exemplary combination immunotherapy protocol.

### DETAILED DESCRIPTION

The foregoing and other aspects of the present disclosure will now be described in more detail with respect to the description and methodologies provided herein. This description is not intended to be a detailed catalogue of all the ways in which the embodiments of the present disclosure may be implemented, or of all the features that may be added to the present disclosure. For example, features illustrated with respect to one embodiment may be incorporated into other embodiments, and features illustrated with respect to a particular embodiment may be deleted from that embodiment. In addition, numerous variations and additions to the various embodiments suggested herein, which do not depart from the instant disclosure, will be apparent to those skilled in the art in light of the instant detailed description, figures and claims. Hence, the following specification is intended to illustrate some particular embodiments, and not to exhaustively specify all permutations, combinations and variations thereof.

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

The present disclosure relates to a multidisciplinary attack on neoplastically transformed cells at various grades and stages through utilizing testing of tumor for sensitivity to tumor-reducing agents, administration/implementation of one or more tumor reduction means, administration of agents targeting of tumor hypoxia, creating a tumor microenvironment conducive to immune mediated killing, administration of immunogens, assessment of immunity, and if needed augmentation of immunity. In some embodiments the methods and compositions disclosed herein utilize a combination of: a) Hypoxia Tumor Targeting (address hypoxic tumors to generate global control of immunosuppression, treatment resistance, tumor growth, and increased metastasis); b) Heat Tumor Immune Microenvironment (systemic immunotherapy relies on immune cells effectively infiltrating cancer cells (“immunologically hot”). To achieve maximum treatment benefit, tumors may be “heated up” or “primed”-which involves injecting specific immune agents directly into the tumor to attract immune cells to cancer; and c) Advanced testing to predict/target immunotherapeutic escape mechanisms; specific immunotherapies upregulate receptors to allow for escape and/or perhaps immunotherapeutic targets which means some combination of agents that work well in some cases, can still adjust and adapt therapy.

The present disclosure also provides means of assessing the sensitivity of cancer stem cells to therapeutic agents ex vivo in order to determine which agents have the most likely change to exert a therapeutic effect when administered in a clinical situation to a cancer patient. The present disclosure provides for the extraction of cancer cells, isolation of cancer stem cells, either through culture means of enrichment and/or using cell selection techniques, and testing cancer inhibitory compounds on these cells. In contrast to approaches that test cancer therapeutics on typical biopsy samples, which represent rapidly growing progeny of cancer stem cells, the methods and compositions of matter disclosed herein provide means of testing sensitivity of cancer stem cells to therapeutics. This approach provides long term remission and/cures since it is addressing the core of the cancer, which is the stem cell.

### Definitions

Although the following terms are believed to be well understood by one of skill in the art, the following definitions are set forth to facilitate understanding of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

As used herein, the terms “a” or “an” or “the” may refer to one or more than one. For example, “a” marker can mean one marker or a plurality of markers.

As used herein, the term “about,” when used in reference to a measurable value such as an amount of mass, dose, time, temperature, and the like, is meant to encompass variations of 20%, 10%, 5%, 1%, 0.5%, or even 0.1% of the specified amount.

As used herein, the term “and/or” refers to and encompasses any and all possible combinations of one or more of the associated listed items, as well as the lack of combinations when interpreted in the alternative (“or”).

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

As used herein, the term “consists essentially of” (and grammatical variants thereof), as applied to the compositions and methods of the present disclosure, means that the compositions/methods may contain additional components so long as the additional components do not materially alter the composition/method.

The term “materially alter,” as applied to a composition/method, refers to an increase or decrease in the effectiveness of the composition/method of at least about 20% or more. For example, a component added to a composition of the present disclosure would “materially alter” the composition if it increases or decreases the composition's ability to inhibit tumor growth by at least 20%.

The term “about” is used to indicate that a value includes the inherent variation of error for the device, the method being employed to determine the value, or the variation that exists among the study subjects.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. Other specific types of cancer include cinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer. In some particular embodiments, the cancer treated is a melanoma. The term “melanoma” is taken to mean a tumor arising from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. In some embodiments, CARs comprise an intracellular activation domain, a transmembrane domain, and an extracellular domain comprising a tumor associated antigen binding region. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (for example, peptides) or from pattern-recognition receptors, such as Dectins. In certain cases, the spacing of the antigen-recognition domain can be modified to reduce activation-induced cell death. In certain cases, CARs comprise domains for additional co-stimulatory signaling, such as CD3.zeta., FcR, CD27, CD28, CD137, DAP10, DAP12 and/or OX40. In some cases, molecules can be co-expressed with the CAR, including co-stimulatory molecules, reporter genes for imaging (for example, for positron emission tomography), gene products that conditionally ablate the T cells upon addition of a pro-drug, homing receptors, chemokines, chemokine receptors, cytokines, and cytokine receptors.

The term “Costimulatory ligand” or “costimulatory molecule” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand can include, but is not limited to, CD7, B7-1 (CD80), B7-2 (CD86), programmed death (PD) L1, PD-L2, 4-1BB ligand, OX40 ligand, inducible costimulatory ligand (ICOS-L), intercellular adhesion molecule (ICAM), CD30 ligand, CD40, CD70, CD83, human leukocyte antigen G (HLA-G), MHC class I chain-related protein A (MICA), MHC class I chain-related protein B (MICB), herpes virus entry mediator (HVEM), lymphotoxin beta receptor, 3/TR6, immunoglobulin-like transcript (ILT) 3, ILT4, an agonist or antibody that binds Toll ligand receptor and a ligand that specifically binds with B7-H3. A co-stimulatory ligand includes, without limitation, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, tumor necrosis factor superfamily member 14 (TNFSF14 or LIGHT), natural killer cell receptor C (NKG2C), B7-H3, and a ligand that specifically binds with CD83.

The term “cytokine”, as used herein, refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature.

The term “expression construct” or “expression cassette” refers to a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

The term “vector” or “construct” (sometimes referred to as a gene delivery system or gene transfer “vehicle”) refers to a macromolecule or complex of molecules comprising a polynucleotide to be delivered to a host cell, either in vitro or in vivo.

The term “plasmid,” a common type of a vector, is an extra-chromosomal DNA molecule separate from the chromosomal DNA that is capable of replicating independently of the chromosomal DNA. In certain cases, it is circular and double-stranded.

The term “origin of replication” (“ori”) or “replication origin” is a DNA sequence, for example, in a lymphotrophic herpes virus, that when present in a plasmid in a cell is capable of maintaining linked sequences in the plasmid and/or a site at or near where DNA synthesis initiates. As an example, an ori for EBV (Ebstein-Barr virus) includes FR sequences (20 imperfect copies of a 30 bp repeat), and preferably DS sequences; however, other sites in EBV bind EBNA-1, for example, Rep* sequences can substitute for DS as an origin of replication (Kirshmaier and Sugden, 1998). Thus, a replication origin of EBV includes FR, DS or Rep* sequences or any functionally equivalent sequences through nucleic acid modifications or synthetic combination derived therefrom. For example, methods of the present disclosure may also use genetically engineered replication origin of EBV, such as by insertion or mutation of individual elements.

The term “gene,” “polynucleotide,” “coding region,” “sequence,” “segment,” “fragment,” or “transgene” that “encodes” a particular protein, is a nucleic acid molecule that is transcribed and optionally also translated into a gene product, for example, a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need be present so long as the selected coding sequence is capable of being replicated, transcribed, and translated in an appropriate host cell.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

The term “enhancer” is meant a nucleic acid sequence that, when positioned proximate to a promoter, confers increased transcription activity relative to the transcription activity resulting from the promoter in the absence of the enhancer domain.

The term “operably linked” or co-expressed” with reference to nucleic acid molecules is meant that two or more nucleic acid molecules (for example, a nucleic acid molecule to be transcribed, a promoter, and an enhancer element) are connected in such a way as to permit transcription of the nucleic acid molecule. “Operably linked” or “co-expressed” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of heterologous molecules.

The term “Homology” refers to the percent of identity between two polynucleotides or two polypeptides. The correspondence between one sequence and another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions that promote the formation of stable duplexes between homologous regions, followed by digestion with single strand-specific nuclease(s), and size determination of the digested fragments. Two DNA, or two polypeptide, sequences are “substantially homologous” to each other when at least about 80%, preferably at least about 90%, and most preferably at least about 95% of the nucleotides, or amino acids, respectively match over a defined length of the molecules, as determined using the methods above.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring cells or artificially modified cells (for example, fusion cells, genetically modified cells, etc.).

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPScs or iPS cells”.

An “embryonic stem(ES) cell” is an undifferentiated pluripotent cell which is obtained from an embryo in an early stage, such as the inner cell mass at the blastocyst stage, or produced by artificial means (for example nuclear transfer) and can give rise to any differentiated cell type in an embryo or an adult, including germ cells (for example sperm and eggs).

“Induced pluripotent stem cells (iPSes or iPS cells)” are cells generated by reprogramming a somatic cell by expressing or inducing expression of a combination of factors (herein referred to as reprogramming factors). iPS cells can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, Klf4, Nanog, and Lin28. In some embodiments, somatic cells are reprogrammed by expressing at least two reprogramming factors, at least three reprogramming factors, at least four reprogramming factors, at least five reprogramming factors, at least six reprogramming factors, or at least seven reprogramming factors to reprogram a somatic cell to a pluripotent stem cell.

“Hematopoietic progenitor cells” or “hematopoietic precursor cells” refers to cells which are committed to a hematopoietic lineage but are capable of further hematopoietic differentiation and include hematopoietic stem cells, multipotential hematopoietic stem cells, common myeloid progenitors, megakaryocyte progenitors, erythrocyte progenitors, and lymphoid progenitors. Hematopoietic stem cells (HSCs) are multipotent stem cells that give rise to all the blood cell types including myeloid (monocytes and macrophages, granulocytes (neutrophils, basophils, eosinophils, and mast cells), erythrocytes, megakaryocytes/platelets, dendritic cells), and lymphoid lineages (T-cells, B-cells, NK-cells) (see for example, Doulatov et al., 2012; Notta et al., 2015). A “multilymphoid progenitor” (MLP) is defined to describe any progenitor that gives rise to all lymphoid lineages (B, T, and NK cells), but that may or may not have other (myeloid) potentials (Doulatov et al., 2010) and is CD45RA.sup.+, /CD10.sup.+/CD7.sup.−. Any B, T, and NK progenitor can be referred to as an MLP. A “common myeloid progenitor” (CMP) refers to CD45RA.sup.−/CD135.sup.+/CD10.sup.−/CD7.sup.− cells that can give rise to granulocytes, monocytes, megakaryocytes and erythrocytes.

“Pluripotent stem cell” refers to a stem cell that has the potential to differentiate into all cells constituting one or more tissues or organs, or preferably, any of the three germ layers: endoderm (interior stomach lining, gastrointestinal tract, the lungs), mesoderm (muscle, bone, blood, urogenital), or ectoderm (epidermal tissues and nervous system).

The term “somatic cell” refers to any cell other than germ cells, such as an egg, a sperm, or the like, which does not directly transfer its DNA to the next generation. Typically, somatic cells have limited or no pluripotency. Somatic cells used herein may be naturally-occurring or genetically modified.

The term “Programming” is a process that alters the type of progeny a cell can produce. For example, a cell has been programmed when it has been altered so that it can form progeny of at least one new cell type, either in culture or in vivo, as compared to what it would have been able to form under the same conditions without programming. This means that after sufficient proliferation, a measurable proportion of progeny having phenotypic characteristics of the new cell type are observed, if essentially no such progeny could form before programming; alternatively, the proportion having characteristics of the new cell type is measurably more than before programming. This process includes differentiation, dedifferentiation and transdifferentiation.

The term “Differentiation” is the process by which a less specialized cell becomes a more specialized cell type. “Dedifferentiation” is a cellular process in which a partially or terminally differentiated cell reverts to an earlier developmental stage, such as pluripotency or multipotency. “Transdifferentiation” is a process of transforming one differentiated cell type into another differentiated cell type. Typically, transdifferentiation by programming occurs without the cells passing through an intermediate pluripotency stage—i.e., the cells are programmed directly from one differentiated cell type to another differentiated cell type. Under certain conditions, the proportion of progeny with characteristics of the new cell type may be at least about 1%, 5%, 25% or more in order of increasing preference.

The term “subject” or “subject in need thereof” refers to a mammal, preferably a human being, male or female at any age that is in need of a cell or tissue transplantation. Typically the subject is in need of cell or tissue transplantation (also referred to herein as recipient) due to a disorder or a pathological or undesired condition, state, or syndrome, or a physical, morphological or physiological abnormality which is amenable to treatment via cell or tissue transplantation.

The term “disruption” or “alteration” of a gene refers to the elimination or reduction of expression of one or more gene products encoded by the subject gene in a cell, compared to the level of expression of the gene product in the absence of the alteration. Exemplary gene products include mRNA and protein products encoded by the gene. Alteration in some cases is transient or reversible and in other cases is permanent. Alteration in some cases is of a functional or full length protein or mRNA, despite the fact that a truncated or non-functional product may be produced. In some embodiments herein, gene activity or function, as opposed to expression, is disrupted. Gene alteration is generally induced by artificial methods, i.e., by addition or introduction of a compound, molecule, complex, or composition, and/or by alteration of nucleic acid of or associated with the gene, such as at the DNA level. Exemplary methods for gene alteration include gene silencing, knockdown, knockout, and/or gene alteration techniques, such as gene editing. Examples include antisense technology, such as RNAi, siRNA, shRNA, and/or ribozymes, which generally result in transient reduction of expression, as well as gene editing techniques which result in targeted gene inactivation or alteration, for example, by induction of breaks and/or homologous recombination. Examples include insertions, mutations, and deletions. The alterations typically result in the repression and/or complete absence of expression of a normal or “wild type” product encoded by the gene. Exemplary of such gene alterations are insertions, frameshift and missense mutations, deletions, knock-in, and knock-out of the gene or part of the gene, including deletions of the entire gene. Such alterations can occur in the coding region, for example, in one or more exons, resulting in the inability to produce a full-length product, functional product, or any product, such as by insertion of a stop codon. Such alterations may also occur by alterations in the promoter or enhancer or other region affecting activation of transcription, so as to prevent transcription of the gene. Gene alterations include gene targeting, including targeted gene inactivation by homologous recombination.

The term “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

The term “immune response” is a response of a cell of the immune system, such as a B cell, or a T cell, or innate immune cell to a stimulus. In one embodiment, the response is specific for a particular antigen (an “antigen-specific response”).

The term “antigen” is a molecule capable of being bound by an antibody or T-cell receptor. An antigen may generally be used to induce a humoral immune response and/or a cellular immune response leading to the production of B and/or T lymphocytes.

The terms “tumor-associated antigen,” “tumor antigen” and “cancer cell antigen” are used interchangeably herein. In each case, the terms refer to proteins, glycoproteins or carbohydrates that are specifically or preferentially expressed by cancer cells.

The term “epitope” is the site on an antigen recognized by an antibody as determined by the specificity of the amino acid sequence. Two antibodies are said to bind to the same epitope if each competitively inhibits (blocks) binding of the other to the antigen as measured in a competitive binding assay. Alternatively, two antibodies have the same epitope if most amino acid mutations in the antigen that reduce or eliminate binding of one antibody reduce or eliminate binding of the other. Two antibodies are said to have overlapping epitopes if each partially inhibits binding of the other to the antigen, and/or if some amino acid mutations that reduce or eliminate binding of one antibody reduce or eliminate binding of the other.

“Adjuvant” refers to a substance that is capable of enhancing, accelerating, or prolonging an immune response when given with a vaccine immunogen. Any adjuvant may be used, for example ABM2, AS01B, AS02, AS02A, Adjumer, Adjuvax, Algammulin, alum, aluminum phosphate, aluminum potassium sulfate, Bordetella pertussis, calcitriol, chitosan, cholera toxin, CpG, dibutyl phthalate, dimethyldioctadecylammonium bromide (DDA), Freund's adjuvant, Freund's complete, Freund's incomplete (IFA), GM-CSF, GMDP, gamma inulin, glycerol, HBSS (Hank's Balanced Salt Solution), Hiltonol, IL-12, IL-2, imiquimod, interferon-gamma, ISCOM, lipid core peptide (LCP), Lipofectin, lipopolysaccharide (LPS), liposomes, MF59, MLP+TDM, monophosphoryl lipid A, Montanide IMS-1313, Montanide ISA 206, Montanide ISA 720, Montanide ISA-51, Montanide ISA-50, nor-MDP, oil-in-water emulsion, P1005 (non-ionic copolymer), Pam3Cys (lipoprotein), Pertussis toxin, poloxamer, QS21, RaLPS, Ribi, saponin, Seppic ISA 720, soybean oil, squalene, Syntex adjuvant formulation (SAF), synthetic polynucleotides (poly IC/poly AU), TiterMax Tomatine, Vaxfectin, XtendIII, or Zymosan.

“Agonist” refers to is a substance which promotes (induces, causes, enhances or increases) the activity of another molecule or a receptor. The term agonist encompasses substances which bind receptor (for example, an antibody, a homolog of a natural ligand from another species) and substances which promote receptor function without binding thereto (for example, by activating an associated protein).

“Antagonist” or “inhibitor” refers to a substance that partially or fully blocks, inhibits, or neutralizes a biological activity of another molecule or receptor.

“Co-administration” refers to administration of two or more agents to the same subject during a treatment period. The two or more agents may be encompassed in a single formulation and thus be administered simultaneously. Alternatively, the two or more agents may be in separate physical formulations and administered separately, either sequentially or simultaneously to the subject. The term “administered simultaneously” or “simultaneous administration” means that the administration of the first agent and that of a second agent overlap in time with each other, while the term “administered sequentially” or “sequential administration” means that the administration of the first agent and that of a second agent does not overlap in time with each other.

“Immune response” refers to any detectable response to a particular substance (such as an antigen or immunogen) by the immune system of a host vertebrate animal, including, but not limited to, innate immune responses (for example, activation of Toll receptor signaling cascade), cell-mediated immune responses (for example, responses mediated by T cells, such as antigen-specific T cells, and non-specific cells of the immune system), and humoral immune responses (for example, responses mediated by B cells, such as generation and secretion of antibodies into the plasma, lymph, and/or tissue fluids). Examples of immune responses include an alteration (for example, increase) in Toll-like receptor activation, lymphokine (for example, cytokine (for example, Th1, Th2 or Th17 type cytokines) or chemokine) expression or secretion, macrophage activation, dendritic cell activation, T cell (for example, CD4+ or CD8+ T cell) activation, NK cell activation, B cell activation (for example, antibody generation and/or secretion), binding of an immunogen (for example, antigen (for example, immunogenic polypolypeptide)) to an MHC molecule, induction of a cytotoxic T lymphocyte (“CTL”) response, induction of a B cell response (for example, antibody production), and, expansion (for example, growth of a population of cells) of cells of the immune system (for example, T cells and B cells), and increased processing and presentation of antigen by antigen presenting cells. The term “immune response” also encompasses any detectable response to a particular substance (such as an antigen or immunogen) by one or more components of the immune system of a vertebrate animal in vitro.

“Treating a cancer”, “inhibiting cancer”, “reducing cancer growth” refers to inhibiting or preventing oncogenic activity of cancer cells. Oncogenic activity can comprise inhibiting migration, invasion, drug resistance, cell survival, anchorage-independent growth, non-responsiveness to cell death signals, angiogenesis, or combinations thereof of the cancer cells.

The terms “cancer”, “cancer cell”, “tumor”, and “tumor cell” are used interchangeably herein and refer generally to a group of diseases characterized by uncontrolled, abnormal growth of cells (for example, a neoplasia). In some forms of cancer, the cancer cells can spread locally or through the bloodstream and lymphatic system to other parts of the body (“metastatic cancer”). “Ex vivo activated lymphocytes”, “lymphocytes with enhanced antitumor activity” and “dendritic cell cytokine induced killers” are terms used interchangeably to refer to composition of cells that have been activated ex vivo and subsequently reintroduced within the context of the current disclosure. Although the word “lymphocyte” is used, this also includes heterogenous cells that have been expanded during the ex vivo culturing process including dendritic cells, NKT cells, gamma delta T cells, and various other innate and adaptive immune cells. As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma. The term “leukemia” is meant broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

The term “carcinoma” refers to a malignant new growth made up of epithelial cells tending to infiltrate the surrounding tissues, and/or resist physiological and non-physiological cell death signals and give rise to metastases. Exemplary carcinomas include, for example, /pindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some particular embodiments, the cancer treated is a melanoma. The term “melanoma” is taken to mean a tumor arising from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma. The term “polypeptide” is used interchangeably with “peptide”, “altered peptide ligand”, and “flourocarbonated peptides.” The term “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

The term “T cell” is also referred to as T lymphocyte, and means a cell derived from thymus among lymphocytes involved in an immune response. The T cell includes any of a CD8-positive T cell (cytotoxic T cell: CTL), a CD4-positive T cell (helper T cell), a suppressor T cell, a regulatory T cell such as a controlling T cell, an effector cell, a naive T cell, a memory T cell, an .alpha.beta.T cell expressing TCR .alpha. and .beta. chains, and a .gamma.delta.T cell expressing TCR .gamma. and .delta. chains. The T cell includes a precursor cell of a T cell in which differentiation into a T cell is directed. Examples of “cell populations containing T cells” include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids. Further, a variety of cell populations containing T cells and derived from hematopoietic cells can be used. These cells may have been activated by cytokine such as IL-2 in vivo or ex vivo. As these cells, any of cells collected from a living body, or cells obtained via ex vivo culture, for example, a T cell population obtained by the method as disclosed herein as it is, or obtained by freeze preservation, can be used. The term “antibody” is meant to include both intact molecules as well as fragments thereof that include the antigen-binding site. Whole antibody structure is often given as H.sub.2L.sub.2 and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity. The variable regions of either H or L chains contains the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure. The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The antibodies disclosed herein may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain. The term “effective amount” or “therapeutically effective amount” means a dosage sufficient to treat, inhibit, or alleviate one or more symptoms of a disease state being treated or to otherwise provide a desired pharmacologic and/or physiologic effect, especially enhancing T cell response to a selected antigen. The precise dosage will vary according to a variety of factors such as subject-dependent variables (for example, age, immune system health, etc.), the disease, and the treatment being administered. The terms “individual”, “host”, “subject”, and “patient” are used interchangeably herein, and refer to a mammal, including, but not limited to, primates, for example, human beings, as well as rodents, such as mice and rats, and other laboratory animals.

The term “treatment regimen” refers to a treatment of a disease or a method for achieving a desired physiological change, such as increased or decreased response of the immune system to an antigen or immunogen, such as an increase or decrease in the number or activity of one or more cells, or cell types, that are involved in such response, wherein said treatment or method comprises administering to an animal, such as a mammal, especially a human being, a sufficient amount of two or more chemical agents or components of said regimen to effectively treat a disease or to produce said physiological change, wherein said chemical agents or components are administered together, such as part of the same composition, or administered separately and independently at the same time or at different times (i.e., administration of each agent or component is separated by a finite period of time from one or more of the agents or components) and where administration of said one or more agents or components achieves a result greater than that of any of said agents or components when administered alone or in isolation.

The term “anergy” and “unresponsiveness” includes unresponsiveness to an immune cell to stimulation, for example, stimulation by an activation receptor or cytokine. The anergy may occur due to, for example, exposure to an immune suppressor or exposure to an antigen in a high dose. Such anergy is generally antigen-specific, and continues even after completion of exposure to a tolerized antigen. For example, the anergy in a T cell and/or NK cell is characterized by failure of production of cytokine, for example, interleukin (IL)-2. The T cell anergy and/or NK cell anergy occurs in part when a first signal (signal via TCR or CD-3) is received in the absence of a second signal (costimulatory signal) upon exposure of a T cell and/or NK cell to an antigen. The term “enhanced function of a T cell”, “enhanced cytotoxicity” and “augmented activity” means that the effector function of the T cell and/or NK cell is improved. The enhanced function of the T cell and/or NK cell, as a non-limiting example, includes an improvement in the proliferation rate of the T cell and/or NK cell, an increase in the production amount of cytokine, or an improvement in cytotoxity. Further, the enhanced function of the T cell and/or NK cell includes cancellation and suppression of tolerance of the T cell and/or NK cell in the suppressed state such as the anergy (unresponsive) state, or the rest state, that is, transfer of the T cell and/or NK cell from the suppressed state into the state where the T cell and/or NK cell responds to stimulation from the outside.

The term “expression” means generation of mRNA by transcription from nucleic acids such as genes, polynucleotides, and oligonucleotides, or generation of a protein or a polypeptide by transcription from mRNA. Expression may be detected by means including RT-PCR, Northern Blot, or in situ hybridization, “Suppression of expression” refers to a decrease of a transcription product or a translation product in a significant amount as compared with the case of no suppression. The suppression of expression herein shows, for example, a decrease of a transcription product or a translation product in an amount of 30% or more, preferably 50% or more, more preferably 70% or more, and further preferably 90% or more.

### Cancer Treatment

Various embodiments of the present disclosure provide a method of treating, reducing the severity of and/or slowing the progression of a tumor in a subject. The method may consist of or may comprise: providing an immunization means which activates effector cells to expand in vivo, followed by administration of a therapeutically effective amount of antigen presenting cells, such as dendritic cells into the local tumor microenvironment, followed by induction of immunogenic tumor cell death, followed by administration of agents, or vaccines capable of eliciting immunosurveillance to prevent tumor relapse, as well as to induce an abscopal effect. In various embodiments, the immune cell is primed against a tumor cell lysate, tumor cell antigen, tumor cell cytokine, and/or stem cell lysate.

In one embodiment the disclosure provides the utilization of cellular vaccines prior to ablative therapy, and/or concurrent with, and/or subsequent to said therapy. Said ablative therapy may be radiotherapy, and/or cryoablation and/or ultrasound mediated ablation and/or hyperthermia ablation and/or surgery. The aim of administering said cellular vaccine is to induce immunity that acts locally and systemically. One of the objectives of administration of said cellular vaccines is to reduce metastasis, another objective is to encapsulate the tumor prior to ablative therapy. Within the practice of the present disclosure, whole cancer cells may be xenogeneic, allogeneic, syngeneic, or autologous to the treatment recipient. Typically they may be treated to make them proliferation incompetent by a technique which preserves preserve their immunogenicity and their metabolic activity. One typically used technique is irradiation or mitomycin C. Typically the same general type of tumor cell is used that the patient bears. For example, a patient suffering from melanoma will typically be administered proliferation incompetent melanoma cells. The cells may express and secrete a cytokine naturally or by transfection with a nucleic acid which directs such expression and secretion. One suitable cytokine is GM-CSF. For example, the tumor cell may express a transgene encoding GM-CSF as described in U.S. Pat. Nos. 5,637,483, 5,904,920, 6,277,368 and 6,350,445, as well as in US Patent Publication No. 20100150946, each of which is expressly incorporated by reference. One example of a GM-CSF-expressing, genetically modified cancer cell for the treatment of pancreatic cancer is described in U.S. Pat. Nos. 6,033,674 and 5,985,290, both of which are expressly incorporated by reference herein. Other cytokines can be used. Suitable cytokines which may be used include cytokines which stimulate dendritic cell induction, recruitment, and/or maturation. Such cytokines include, but are not limited to, one or more of GM-CSF, CD40 ligand, IL-12, CCL3, CCL20, and CCL21. Granulocyte-macrophage colony stimulating factor (GM-CSF) polypeptide is a cytokine or fragment having immunomodulatory activity and having at least about 85% amino acid sequence identity to GenBank Accession No. AAA52122.1.

In some embodiments immunogenicity of the tumor is increased by administration of “danger” signals. These may come in the form of toll like receptors (TLR), are a family of proteins that sense a microbial product and/or initiates an adaptive immune response. TLR activate a dendritic cell (DC). TLRs are conserved membrane spanning molecules containing an ectodomain of leucine-rich repeats, a transmembrane domain and an intracellular TIR. (Toll/IL-1R) domain. TLRs recognize distinct structures in microbes; often referred to as “PAMPs” (pathogen associated molecular patterns). Ligand binding to TLRs invokes a cascade of intra-cellular signaling pathways that induce the production of factors involved in inflammation and immunity.

Exemplary agonists which may be used for these receptors include, without limitation lipoproteins, lipopolypeptides, peptidoglycans, zymosan, lipopolysaccharide; neisserial porins, flagellin, profillin, galactoceramide, muramyl dipeptide, glucopyranosyl lipid A (GLA), and resiquimod (R848). Peptidoglycans, lipoproteins, and lipoteichoic acids are cell wall components of Gram-positive. Lipopolysaccharides are expressed by most bacteria. Flagellin is the structural component of bacterial flagella that is secreted by pathogenic and commensal bacterial. A Galactosylceramide (α-GalCer) is an activator of natural killer T (NKT) cells. Muramyl dipeptide is a bioactive peptidoglycan motif common to all bacteria. Such agonists mediate innate immune activation via Toll-like Receptors. Specific binding of an agonist for its cognate receptor is often expressed in terms of an affinity. The ligands of the present disclosure may bind with affinities of between about 104 M−1 and about 108 M−1. Affinity is calculated as K_{d}=K_{off}/kₒₙ (k_{off} is the dissociation rate constant, Kₒₙ is the association rate constant and K_{d} is the equilibrium constant). Single or multiple agonists may be used.

In humans, ten TLR have been identified. TLRs that are expressed on the surface of cells include TLR-1, -2, -4, -5, and -6, while TLR-3, -7/8, and -9 are expressed with the ER compartment. Human dendritic cell subsets can be identified on the basis of distinct TLR expression patterns. By way of example, the myeloid or “conventional” subset of DC (mDC) expresses TLRs 1-8 when stimulated, and a cascade of activation markers (for example CD80, CD86, WIC class I and II, CCR7), pro-inflammatory cytokines, and chemokines are produced. A result of this stimulation and resulting expression is antigen-specific CD4+ and CD8+ T cell priming. These DCs acquire an enhanced capacity to take up antigens and present them in an appropriate form to T cells. In contrast, the plasmacytoid subset of DC (pDC) expresses only TLR7 and TLR9 upon activation, with a resulting activation of NK cells as well as T-cells. As dying tumor cells may adversely affect DC function, it has been suggested that activating DC with TLR agonists may be beneficial for priming anti-tumor immunity in an immunotherapy approach to the treatment of cancer. It has also been suggested that successful treatment of breast cancer using radiation and chemotherapy requires TLR4 activation.

In one embodiment of the present disclosure, immunization to tumors of the same type the patient is suffering from is provided prior to cytotoxic, or immunogenic cell death induction of the tumor. Immunization of the patient may be performed using known means in the art, using suitable adjuvants. Assessment of immunity is performed by quantifying reactivity of T cells or B cells in response to protein antigens or derivatives thereof, derivatives including peptide antigens or other antigenic epitopes. Responses may be assessed in terms of proliferative responses, cytokine release, antibody responses, or generation of cytotoxic T cells. Methods of assessing said responses are well known in the art. In a preferred embodiment, antibody responses are assessed to a panel of tumor associated proteins subsequent to immunization of patient. Antibody responses are utilized to guide which peptides will be utilized for prior immunization. For example, if a patient is immunized with tumor antigen on a weekly basis, the subsequent assessment of antibody responses is performed at approximately 1-3 months after initiation of immunization. Protocols for immunization include weekly, biweekly, or monthly. Assessment of antibody responses is performed utilizing standard enzyme linked immunosorbent (ELISA) assay. Assessment of antibodies is performed, in one embodiment of the present disclosure, against proteins associated with tumor.

In some embodiments, culture of the immune effectors cells is performed after extracting from a patient that has been immunized with a polyvalent antigenic preparation. Specifically separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used. In some embodiments of the present disclosure, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-.gamma., transforming growth factor (TGF)-.beta., IL-15, IL-7, IFN-.alpha., IL-12, CD40L, and IL-27. From the viewpoint of enhancing cellular immunity, particularly suitably, IL-2, IFN-gamma., or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1.alpha., MIP1.beta., CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the present disclosure to optimize the cellular product based on other means of assessing T cell activity, for example, the function enhancement of the T cell in the method of the present disclosure can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the present disclosure may be assessed in a living body before first administration of the T cell with enhanced function of the present disclosure, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects. Said cells can be expanded in the presence of specific antigens associated with tumors and subsequently injected into the patient in need of treatment. Expansion with specific antigens includes coculture with proteins selected from a group comprising of: a) ROBO; b) VEGF-R2; c) FGF-R; d) CD105; c) TEM-1; and f) survivin. Other tumor specific or semi-specific antigens are known in the art that may be used.

Within the context of the present disclosure, teachings are provided to amplify an antigen specific immune response following immunization with a polyvalent vaccine, in which the antigenic epitopes are used for immunization together with adjuvants such as toll like receptors (TLRs). These molecules are type 1 membrane receptors that are expressed on hematopoietic and non-hematopoietic cells. At least 11 members have been identified in the TLR family. These receptors are characterized by their capacity to recognize pathogen-associated molecular patterns (PAMP) expressed by pathogenic organisms. It has been found that triggering of TLR elicits profound inflammatory responses through enhanced cytokine production, chemokine receptor expression (CCR2, CCR5 and CCR7), and costimulatory molecule expression. As such, these receptors in the innate immune systems exert control over the polarity of the ensuing acquired immune response. Among the TLRs, TLR9 has been extensively investigated for its functions in immune responses. Stimulation of the TLR9 receptor directs antigen-presenting cells (APCs) towards priming potent, T.sub.H1-dominated T-cell responses, by increasing the production of pro-inflammatory cytokines and the presentation of co-stimulatory molecules to T cells. CpG oligonucleotides, ligands for TLR9, were found to be a class of potent immunostimulatory factors. CpG therapy has been tested against a wide variety of tumor models in mice, and has consistently been shown to promote tumor inhibition or regression.

In some embodiments, specific antigens are immunized following polyvalent immunization, said specific antigens administered in the form of DNA vaccines. Numerous publications have reported animal and clinical efficacy of DNA vaccines which are incorporated by reference [1-3]. In addition to direct DNA injection techniques, DNA vaccines can be administered by electroporation [4]. The nucleic acid compositions, including the DNA vaccine compositions, may further comprise a pharmaceutically acceptable excipient. Examples of suitable pharmaceutically acceptable excipients for nucleic acid compositions, including DNA vaccine compositions, are well known to those skilled in the art and include sugars, etc. Such excipients may be aqueous or non aqueous solutions, suspensions, and emulsions. Examples of non-aqueous excipients include propylene glycol, polyethylene glycol, vegetable oils such as olive oil, and injectable organic esters such as ethyl oleate. Examples of aqueous excipient include water, alcoholic/aqueous solutions, emulsions or suspensions, including saline and buffered media. Suitable excipients also include agents that assist in cellular uptake of the polynucleotide molecule. Examples of such agents are (i) chemicals that modify cellular permeability, such as bupivacaine, (ii) liposomes or viral particles for encapsulation of the polynucleotide, or (iii) cationic lipids or silica, gold, or tungsten microparticles which associate themselves with the polynucleotides. Anionic and neutral liposomes are well-known in the art (see, for example, Liposomes: A Practical Approach, RPC New Ed, IRL press (1990), for a detailed description of methods for making liposomes) and are useful for delivering a large range of products, including polynucleotides. Cationic lipids are also known in the art and are commonly used for gene delivery. Such lipids include Lipofectin.™. also known as DOTMA (N—[I-(2,3-dioleyloxy) propyls N,N,N-trimethylammonium chloride), DOTAP (1,2-bis(oleoyloxy)-3 (trimethylammonio) propane), DDAB (dimethyldioctadecyl-ammonium bromide), DOGS (dioctadecylamidoglycyl spermine) and cholesterol derivatives such as DCChol (3 beta-(N—(N′,N′-dimethyl aminomethane)-carbamoyl) cholesterol). A description of these cationic lipids can be found in EP 187,702, WO 90/11092, U.S. Pat. No. 5,283,185, WO 91/15501, WO 95/26356, and U.S. Pat. No. 5,527,928. A particular useful cationic lipid formulation that may be used with the nucleic vaccine provided by the disclosure is VAXFECTIN, which is a commixture of a cationic lipid (GAP-DMORIE) and a neutral phospholipid (DPyPE) which, when combined in an aqueous vehicle, self-assemble to form liposomes. Cationic lipids for gene delivery are preferably used in association with a neutral lipid such as DOPE (dioleyl phosphatidylethanolamine), as described in WO 90/11092 as an example. In addition, a DNA vaccine can also be formulated with a nonionic block copolymer such as CRL1005. Other immunization means include prime boost regiments [5]. The polypeptide and nucleic acid compositions can be administered to an animal, including human, by a number of methods known in the art. Examples of suitable methods include: (1) intramuscular, intradermal, intraepidermal, intravenous, intraarterial, subcutaneous, or intraperitoneal administration, (2) oral administration, and (3) topical application (such as ocular, intranasal, and intravaginal application). One particular method of intradermal or intraepidermal administration of a nucleic acid vaccine composition that may be used is gene gun delivery using the Particle Mediated Epidermal Delivery (PMED.™) vaccine delivery device marketed by PowderMed [6]. PMED is a needle-free method of administering vaccines to animals or humans. The PMED system involves the precipitation of DNA onto microscopic gold particles that are then propelled by helium gas into the epidermis [7]. The DNA-coated gold particles are delivered to the APCs and keratinocytes of the epidermis, and once inside the nuclei of these cells, the DNA elutes off the gold and becomes transcriptionally active, producing encoded protein. This protein is then presented by the APCs to the lymphocytes to induce a T-cell-mediated immune response. Another particular method for intramuscular administration of a nucleic acid vaccine provided by the present disclosure is electroporation [8]. Electroporation uses controlled electrical pulses to create temporary pores in the cell membrane, which facilitates cellular uptake of the nucleic acid vaccine injected into the muscle [9-12]. Where a CpG is used in combination with a nucleic acid vaccine, it is preferred that the CpG and nucleic acid vaccine are co-formulated in one formulation and the formulation is administered intramuscularly by electroporation. A helper T cell and cytotoxic T cell stimulatory polypeptide can be introduced into a mammalian host, including humans, linked to its own carrier or as a homopolymer or heteropolymer of active polypeptide units. Such a polymer can elicit increase immunological reaction and, where different polypeptides are used to make up the polymer, the additional ability to induce antibodies and/or T cells that react with different antigenic determinants of the tumor. Useful carriers known in the art include, for example, thyroglobulin, albumins such as human serum albumin, tetanus toxoid, polyamino acids such as poly(D-lysine: D-glutamic acid), influenza polypeptide, and the like. Adjuvants such as incomplete Freunds adjuvant, GM-CSF, aluminum phosphate, CpG containing DNA, inulin, Poly (IC), aluminum hydroxide, alum, or montanide can also be used in the administration of an helper T cell and cytotoxic T cell stimulatory polypeptide.

Subsequent to augmentation of lymphocyte numbers specific for killing of the tumor, modification of the tumor microenvironment may be performed. In one embodiment, macrophage modulators are used.

Macrophages are key components of the innate immune system which play a principal role in the regulation of inflammation as well as physiological processes such as tissue remodeling [13, 14]. The diverse role of macrophages can be seen in conditions ranging from wound healing [15-18], to myocardial infarction [19-25], to renal failure [26-29] and liver failure [30].

Differentiated macrophages and their precursors are versatile cells that can adapt to microenvironmental signals by altering their phenotype and function [31]. Although they have been studied for many years, it has only recently been shown that these cells comprise distinct sub-populations, known as classical M1 and alternative M2 [32]. Mirroring the nomenclature of Th1 cells, M1 macrophages are described as the pro-inflammatory sub-type of macrophages induced by IFN-.gamma. and LPS. They produce effector molecules (for example, reactive oxygen species) and pro-inflammatory cytokines (for example, IL-12, TNF-.alpha. and IL-6) and they trigger Th1 polarized responses [33].

Macrophages can play a tumor inhibitory, as well as a tumor stimulatory role. Initial studies supported the role of macrophages in mediating antibody dependent cellular cytotoxicity in tumors [34-41], and thus being associated with potentiation of antitumor immune responses. Macrophages also possess the ability to directly recognize tumors by virtue of tumor expressed “eat-me” signals, which include the stress associated protein calreticulin [42, 43], which binds to the low-density lipoprotein receptor-related protein (LRP) on macrophages to induce phagocytosis [44]. Tumors protect themselves by expression of CD47, which binds to macrophage SIRP-1 and transduces an inhibitory signal [45]. Blockade of CD47 using antibodies results in remission of cancers mediated by macrophage activation [46-50]. Thus on the one hand, macrophages play an important role in induction of antitumor immunity. This can also be exemplified by some studies, involving administration of GM-CSF in order to augment macrophage numbers and activity in cancer patients [51-54].

Unfortunately, there is also evidence that macrophages support tumor growth. Studies in the osteopetrotic mice strain, which lacks mature macrophages, demonstrate that tumors actually grow slower in animals deficient in macrophages [55]. Several other animal models have elegantly demonstrated that macrophages contribute to tumor growth, in part through stimulating on the angiogenic switch [56-58]. Numerous tumor biopsy studies have shown that there is a negative correlation between macrophage infiltration into tumors and patient survival [59-63].

The duality of macrophages in growth of tumors may be seen in studies of “inverse hormesis” in which low concentrations of antibodies targeting the tumor specific marker sialic acid N-glycolyl-neuraminic acid (Neu5Gc) actually leads to enhanced tumor growth in a macrophage dependent manner [64].

The importance of macrophages in clinical implementation of cancer therapeutics can be seen from results of a double blind clinical trials in metastatic colorectal cancer patients where cetuximab (anti-epidermal growth factor receptor (EGFR) monoclonal antibody (mAb)) was added to a protocol comprising of bevacizumab and chemotherapy. The addition of cetuximab actually resulted in decreased survival. In a study examining whether monocyte conversion to M2 angiogenic macrophages was responsible, investigators observed that CD163-positive M2 macrophages where found in high concentrations intratumorally in patients with colorectal carcinomas. These M2 cells expressed abundant levels of Fc-gamma receptors (FcγR) and PD-L1. Additionally, consistent with the M2 phenotype the cells generated large amounts of the immunosuppressive molecule IL-10 and the angiogenic mediator VEGF. When M2 cells were cultured with EGFR-positive tumor cells loaded with low concentrations of cetuximab, further augmentation of IL-10 and VEGF production was observed. These data suggest that under certain contexts, tumors manipulate macrophages to take on the M2 phenotype, and this subsequently leads to enhanced tumor progressing factors when tumor cells are bound by antibodies [65].

Manipulation of macrophages to inhibit M2 and shift to M1 phenotype may be performed using a variety of means. One theme that seems unifying is the ability of toll like receptor (TLR) agonists to influence this. In addition to cytokine differences, macrophages capable of killing tumor cells are usually known to express low levels of the inhibitory Fc gamma receptor IIb, whereas tumor promoting macrophages have high levels of this receptor [66]. Furthermore, tumor associated cytokines such as IL-4 and IL-10 are known to induce upregulation of the Fc gamma receptor IIB [67-70].

In one study, the effect of the TLR7/8 agonist R-848 was assessed on monocytes derived from human peripheral blood. It was found that 12 hour exposure of R-848 increased FcgammaR-mediated cytokine production and antibody-dependent cellular cytotoxicity by monocytes. Furthermore, upregulation of the ADCC associated receptors FcgammaRI, FcgammaRIIa, and the common gamma-subunit was observed. However, treatment with R-848 led to profound downregulation of the inhibitory FcgammaRIIb molecule [71]. These data support ability to modify therapeutic activity of macrophages by manipulation of TLR signaling pathways. Other TLRs have been found to suppress inhibitory receptors on macrophages. For example, in another study it was observed that exposing monocytes to TLR4 agonists leads to suppression of the FcγRIIb macrophage inhibitory protein by MARCH3 mediated ubiquitination [72].

In one embodiment administration of ImmunoMax is performed systemically, and/or locally, which is an injectable polysaccharide purified from potato sprouts and approved as pharmaceutical in the Russian Federation (registration P No. 001919/02-2002) and 5 other countries of Commonwealth of Independent States (formerly the USSR) and has been evaluated in a wide range of medical situations. In accordance with the formal “Instruction of Medical Use”, one medical indication for Immunomax® is the stimulation of immune defense during the treatment of different infectious diseases (gepon.ru/immax_intro.htm). Studies have shown that Immunomax® induces immune mediated killing of cancer cells in a TLR4 dependent manner [73]. In some embodiments, ImmunoMax is utilized to induce an M2 to M1 shift, thus reducing macrophage derived immune suppressants and augmenting production of immune stimulatory cytokines such as IL-12 and TNF-alpha [73]. In some embodiments, other agents may be used to modulate M2 to M1 transition of tumor associated macrophages including RRx-001 [74], the bee venom derived peptide melittin [75], CpG DNA [76, 77], metformin [78], Chinese medicine derivative puerarin [79], rhubarb derivative emodin [80], dietary supplement chlorogenic acid [81], propranolol [82], poly ICLC [83], BCG [84], Agaricus blazei Murill mushroom extract [85], endotoxin [86], olive skin derivative maslinic acid [87], intravenous immunoglobulin [88], phosphotidylserine targeting antibodies [89], dimethyl sulfoxide [90], surfactant protein A [91], Zoledronic acid [92], bacteriophages [93],

Prior to induction of immunogenic cell death, antigen presenting cells are administered, one of the most potent antigen presenting cells is the dendritic cell.

Dendritic cells (DC) possess unique morphology similar to neuronal dendrites and were originally identified based on their ability to stimulate the adaptive immune system. Of importance to the field of tumor immunotherapy, dendritic cells appear to be the only cell in the body capable of activating naïve T cells [94]. The concept of dendritic cells instructing naïve T cells to differentiate into effector or memory cells is fundamental because it places the dendritic cell as the most powerful antigen presenting cell. This implies that for immunotherapeutic purposes dendritic cells do not necessarily need to be administered at high numbers in patients. One way in which dendritic cells have been described is as sentinels of the immune system that are patrolling the body in an immature state [95, 96]. Once DC are activated, by a stimulatory signal such as a Damage Associated Molecular Patterns (DAMPS) the DC then migrate into the draining lymph nodes through the afferent lymphatics. During the trafficking process, DC degrade ingested proteins into peptides that bind to both MHC class I molecules and MHC class II molecules. This allows the DC to: a) perform cross presentation in that they ingest exogenous antigens but present peptides in the MHC I pathway; and b) activate both CD8 (via MHC I) and CD4 (via MHC II). Interestingly, lipid antigens are processed via different pathways and are loaded onto non-classical MHC molecules of the CD1 family [97].

The possibility of utilizing DC to stimulate immunity was made into reality in animal studies that took advantage of the ability of immature DC to potently phagocytose various antigens. If the antigens possessed DAMPs, or if DAMPs were present in the environment, the DC would mature and present the antigens, resulting in stimulation of potent T cell immunity. Accordingly, in the initial studies, immature DC were incubated with various antigens, subsequent to which a maturation signal (replicating natural DAMPs) was applied and the DC were injected into animals. Thus, DC were utilized as a type of “cellular adjuvant”. Indeed, it was discovered that the classical adjuvants such as Freynd's Adjuvant actually contained a high concentration of DAMPs, which resulted in the stimulation of local DC at vaccination site in vivo.

One of the first clinical applications of DC was prostate cancer. In an early reported, thirty three androgen resistant metastatic prostate cancer patients were treated with DC that were pulsed with peptides from a prostate specific antigen termed PMSA. Nine partial responders were identified based on NCPC criterial, plus 50% reduction of PSA. Four of the partial responders were also responders in the phase I study, with an average response duration of 225 days. Their combined average total response period was over 370 days. Five other responders in the secondary immunizations at the Phase II were nonresponders in the phase I study. Their average partial response period was 196 days. These data support the safety of follow-up infusion of DC that have been pulsed with tumor antigen derived peptide [98].

The same group published a subsequent paper on an additional 33 patients that had not received prior DC immunization in the Phase I. All subjects received six infusions of DC pulsed with PSM-P1 and -P2 at six week intervals without any treatment associated adverse events. Six partial and two complete responders were identified in the phase II study based on NPCP criteria, plus 50% reduction of prostate-specific antigen (PSA), or resolution in previously measurable lesions on ProstaScint scan [99]. The same group analyzed immune response in patients who had clinical remission or relapsed. A strong correlation was found between delayed type hypersensitivity response to the PSM-P1 and PSM-P2 and clinical response [100].

Another subsequent study utilized DC generated using GM-CSF and IL-4 but pulsed with PAP, another prostate antigen. Specifically, the PAP was delivered to the DC by means of generation of a PAP-GM-CSF fusion protein. Two intravenous infusions of the generated cells were performed one month apart in 12 patients with androgen resistant prostate cancer. The infusions were followed by three s.c. monthly doses of the fusion protein without cells. Treatment was well tolerated and circulating prostate-specific antigen levels dropped in three patients. Immune response to the fusion protein was observed, as well as to PAP [101]. In addition to prostate cancer, in which FDA approval has been granted for the Provenge drug, numerous trials have been conducted in a wide variety of cancers. All the trials demonstrated safety, without serious adverse effects of DC administration, as well as some degree of therapeutic efficacy. Trials have been conducted in melanoma [102-153], soft tissue sarcoma, thyroid [155-157], glioma [158-179], multiple myeloma, [180-188], lymphoma [189-191], leukemia [192-199], as well as liver [200-205], lung [206-219], ovarian [220-223], and pancreatic cancer [224-226].

Within the context of the present disclosure, T cell activation is performed in vivo. In one embodiment, transfer factor is utilized. T cells are immune effectors against tumors, possessing ability to directly kill via CD8 cytotoxic cells [227-229], or indirectly killing tumors by activation of macrophages through interferon gamma production [230-232]. Additionally, T cells have been shown to convert protumor M2 macrophages to M1 [233]. The importance of T cells in cancer is illustrated by positive correlation between tumor infiltrating lymphocytes and patient survival [234-238]. In addition, positive correlations between responses to various immunotherapies has been made with tumor infiltrating lymphocyte density [239, 240]. Increased T cell activity is associated with reduction in T regulatory (Treg) cells. Studies show that agents that cause suppression of Treg cells correlates with improved tumor control. Agents that inhibit Treg cells include arsenic trioxide [241], cyclophosphamide [242-244], triptolide [243], gemcitabine [245], and artemether [246].

T cell modulator (TCM) is a pharmaceutical grade transfer factor, which activates T cells by reducing costimulatory requirements, thus potentially increasing infiltration of tumors by T cells. The concept of an immunologically acting “Transfer Factor” was originally identified by Henry Lawrence in a 1956 publication [247], in which he reported simultaneous transfer of delayed hypersensitivity to diphtheria toxin and to tuberculin in eight consecutive healthy volunteers who received extracts from washed leucocytes taken from the peripheral blood of tuberculin-positive, Schick-negative donors who were highly sensitive to purified diphtheria toxin and toxoid. The leucocyte extracts used for transfer contained no detectable antitoxin. The recipient subjects were Schick-positive (<0.001 unit antitoxin per ml. serum) and tuberculin-negative at the time of transfer. All the recipients remained Schick-positive for at least 2 weeks following transfer and in every case their serum contained less than 0.001 units antitoxin at the time when they exhibited maximal skin reactivity to toxoid. The “transfer factor” that was utilized was prepared by washing packed leukocytes isolated using the bovine fibrinogen method, and washing the leukocytes twice in recipient plasma. The washed leukocytes were subsequently lysed by 7-10 freeze-thaw cycles in the presence of DNAse with Mg++. Administration of the extract was performed intradermally and subcutaneously over the deltoid area.

Given that in those early days little was known regarding T cell specificity and MHC antigen presentation, the possibility that immunological information was transmitted by these low molecular weight transfer factors was taken seriously. Transfer factors of various sizes and charges were isolated, with some concept that different antigens elicited different types of transfer factors [248, 249]. Numerous theories were proposed to the molecular nature of transfer factor. Some evidence was that it constituted chains of antibodies that were preformed but subsequently cleaved [250]. Functionally, one of the main thoughts was that transfer factor has multiple sites of action, including effects on the thymus, on lymphocyte-monocyte and/or lymphocyte-lymphocyte interactions, as well as direct effects on cells in inflammatory sites. It is also suggested that the “specificity” of transfer factor is determined by the immunologic status of the recipient rather than by informational molecules in the dialysates [251].

Burger et al [252], used exclusion chromatography to perform characterization of transfer factor. The found that specific transferring ability of transfer factor in vivo was found to reside in the major UV-absorbing peak (Fraction III). Fraction III transferred tuberculin, candida, or KLH-reactivity to previously negative recipients. Fraction III from nonreactive donors was ineffective. When the fractions were tested in vitro, we found that both the mitogenic activity of whole transfer factor and the suppressive activity to mitogen activation when present in transfer factor was found in Fraction I. Fraction III contained components responsible for augmentation of PHA and PWM responses. In addition, Fraction III contained the component responsible for antigen-dependent augmentation of lymphocyte transformation. Fraction IV was suppressive to antigen-induced lymphocyte transformation.

In 1992 Kirkpatrick characterized the specific transfer factor at molecular level. The transfer factor is constituted by a group of numerous molecules, of low molecular weight, from 1.0 to 6.0 kDa. The 5 kDa fraction corresponds to the transfer factor specific to antigens. There are a number of publications about the clinical indications of the transfer factor for diverse diseases, in particular those where the cellular immune response is compromised or in those where there is a deficient regulation of the immune response. It has been demonstrated that the transfer factor increases the expression of IFN-gamma and RANTES, while decreases the expression of osteopontin. Using animal models, it has been reported that transfer factor possesses activity against M. tuberculosis, and with a model of glioma with good therapeutic results. In the clinical setting studies have reported effects against herpes zoster, herpes simplex type I, herpetic keratitis, atopic dermatitis, osteosarcoma, tuberculosis, asthma, post-herpetic neuritis, anergic coccidioidomycosis, leishmaniasis, toxoplasmosis, mucocutaneous candidiasis, pediatric infections produced by diverse pathogen germs, sinusitis, pharyngitis, and otitis media. All of these diseases were studied through protocols which main goals were to study the therapeutic effects of the transfer factor, and to establish in a systematic way diverse dosage schema and time for treatment to guide the prescription of the transfer factor [253].

In some embodiments, administration of intravenous vitamin C is utilized. Patients treated with immunotherapy have been shown to develop a scurvy-like condition. The patient presented with acute signs and symptoms of scurvy (perifollicular petechiae, erythema, gingivitis and bleeding). Serum ascorbate levels were significantly reduced to almost undetectable levels [254]. Although the role of ascorbic acid (AA) hypersupplementation in stimulation of immunity in healthy subjects is controversial, it is well established that AA deficiency is associated with impaired cell mediated immunity. This has been demonstrated in numerous studies showing deficiency suppresses T cytotoxic responses, delayed type hypersensitivity, and bacterial clearance [255]. Additionally, it is well-known that NK activity, which IL-2 is anti-tumor activity is highly dependent on, is suppressed during conditions of AA deficiency [256]. Thus, it may be that while IL-2 therapy on the one hand is stimulating T and NK function, the systemic inflammatory syndrome-like effects of this treatment may actually be suppressed by induction of a negative feedback loop. Such a negative feedback loop with IL-2 therapy was successfully overcome by work using low dose histamine to inhibit IL-2 mediated immune suppression, which led to the “drug” Ceplene (histamine dichloride) receiving approval as an IL-2 adjuvant for treatment of AML [257].

The concept of AA deficiency subsequent to IL-2 therapy was reported previously by another group. Marcus et al evaluated 11 advanced cancer patients suffering from melanoma, renal cell carcinoma and colon cancer being on a 3 phase immunotherapeutic program consisting of: a) 5 days of i.v. high-dose (10(5) units/kg every 8 h) interleukin 2, (b) 6½ days of rest plus leukapheresis; and (c) 4 days of high-dose interleukin 2 plus three infusions of autologous lymphokine-activated killer cells. Mean plasma ascorbic acid levels were normal (0.64+/−0.25 mg/dl) before therapy. Mean levels dropped by 80% after the first phase of treatment with high-dose interleukin 2 alone (0.13+/−0.08 mg/dl). Subsequently plasma ascorbic acid levels remained severely depleted (0.08 to 0.13 mg/dl) throughout the remainder of the treatment, becoming undetectable (less than 0.05 mg/dl) in eight of 11 patients during this time. Importantly, blood pantothenate and plasma vitamin E remained within normal limits in all 11 patients throughout the phases of therapy, suggesting the hypervitaminosis was specific AA. Strikingly, Responders (n=3) differed from nonresponders (n=8) in that plasma ascorbate levels in the former recovered to at least 0.1 mg/dl (frank clinical scurvy) during Phases 2 and 3, whereas levels in the latter fell below this level [258]. Similar results were reported in another study by the same group examining an additional 15 patients [259]. The possibility that prognosis was related to AA levels is intriguing because of the possibility of higher immune response in these patients, however this has not been tested.

The state of AA deficiency in cancer patients, whether or not as a result of inflammation, suggests supplementation may yield benefit in quality of life. [260]. Improvements in quality of life were also noted in the early studies of Murata et al [261], as well as Cameron [262]. But in addition to this endpoint there appears to be a growing number of studies suggesting direct anti-cancer effects via generation of free radicals locally at tumor sites [263]. In vitro studies on a variety of cancer cells including neuroblastoma [264], bladder cancer [265], pancreatic cancer [266], mesothelioma [267], hepatoma [268], have demonstrated cytotoxic effects at pharmacologically achievable concentrations.

Enhancement of cytotoxicity of Docetaxel, Epirubicin, Irinotecan and 5-FU to a battery of tumor cell lines by AA was demonstrated in vitro [269]. In vivo studies have also supported the potential anticancer effects of AA. For example, Pollard et al used the rat PAIII androgen-independent syngeneic prostate cancer cell line to induce tumors in Lobund-Wistar rats. Daily intraperitoneal administration of AA for 30 days, with evaluation at day 40 revealed significant inhibition of tumor growth, as well as reduction in pulmonary and lymphatic metastasis [270]. Levine's group reported successful in vivo inhibition of human xenografted glioma, ovarian, and neuroblastoma cells in immune deficient animals by administration of AA. Interestingly control fibroblasts were not affected [271]. Clinical reports of remission induced by IV AA have been published [272], however, as mentioned above, formal trials are still ongoing.

In addition to direct cytotoxicity of AA on tumor cells, inhibition of angiogenesis may be another mechanism of action. It has been reported that AA inhibits HUVEC proliferation in vitro [273], as well as suppressing neovascularization in the chorionic allontoic membrane assay [274]. Recently we have reported that in vivo administration of AA results in suppressed vascular cord formation in mouse models [275]. Supporting this possibility, Yeom et al demonstrated that parenteral administration of AA in the S-180 sarcoma model leads to reduced tumor growth, which was associated with suppression of angiogenesis and the pro-angiogenic factors bFGF, VEGF, and MMP-2 [276]. Recent studies suggest that AA suppresses activation of the hypoxia inducible factor (HIF)-1, which is a critical transcription factor that stimulates tumor angiogenesis [277-279]. The clinical relevance of this has been demonstrated in a study showing that endometrial cancer patients having reduced tumor ascorbate levels possess higher levels of HIF-1 activation and a more aggressive phenotype [280].

Thus the possibility exists that administration of AA for treatment of tumor inflammatory mediated pathologies may also cause an antitumor effect. Whether this effect is mediated by direct tumor cytotoxicity or inhibition of angiogenesis remains to be determined. Unfortunately, none of the ongoing trials of AA in cancer patients seek to address this issue [281-286].

Despite numerous claims in the popular media and even on vitamin labels, the concept of AA stimulating immunity is not as clear-cut. Part of this is because ROS are involved in numerous signals of immune cells [287]. For example, it is known that T cell receptor signaling induces an intracellular flux of ROS which is necessary for T cell activation [288]. There are numerous studies demonstrating ascorbic acid under certain conditions actually can inhibit immunity. For example, high dose ascorbate inhibits T cell and B cell proliferative responses as well as IL-2 secretion in vitro [289, 290], as well as NK cytotoxic activity [291]. Additionally, AA has been demonstrated to inhibit T cell activating ability of dendritic cells by rendering them in an immature state in part through inhibition of NF-kappa B [292].

However, it appears that the immune stimulatory effects of AA are actually observed in the context of background immune suppression or in situations of AA deficiency, both of which are well-known in the cancer and SIRS patient. A common occurrence in cancer [293-297] and SIRS patients [298, 299] is the presence of a cleaved T cell receptor (TCR) zeta chain. The zeta chain is an important component of T cell and NK cell activation, that bears the highest number of immunoreceptor tyrosine-based activation motifs (ITAMs) of other TCR and NK signaling molecules [300]. At a cellular level cleavage of the zeta chain is associated with loss of T/NK cell function and spontaneous apoptosis [301-303], at a clinical level it is associated with poor prognosis [304-309].

Since loss of TCR zeta chain is found in other inflammatory conditions ranging from hemodialysis [310, 311], to autoimmunity [312-315], to heart disease [316], the possibility that inflammatory mediators such as ROS cause TCR zeta downregulation has been suggested. Circumstantial evidence comes from studies associated inflammatory cells such as tumor associated macrophages (TAMS) with suppression of zeta chain expression [317]. Myeloid suppressor cells, which are known to produce high concentrations of ROS [318-320] have also been demonstrated to induce reduction of TCR zeta chain in cancer [321], and post trauma [322]. Administration of anti-oxidants has been shown to reverse TCR zeta chain cleavage in tissue culture [323, 324]. Therefore, from the T cell side of immunity, an argument could be made that intravenous ascorbic acid may upregulate immunity by blocking zeta chain downregulation in the context of cancer and acute inflammation.

While it is known that AA functions as an antioxidant in numerous biological conditions, as well as reduces inflammatory markers, the possibility that AA actually increases immune function in cancer patients, as well as is effects on survival and other cancer-related events, has never been formally tested. IV AA has a long and controversial history in relation to reducing tumors in patients. This has impeded research into other potential benefits of this therapy in cancer patients such as reduction of inflammation, improvement of quality of life, and impeding SIRS initiation and progression to MOF. While ongoing clinical trials of IV AA for cancer may or may not meet the bar to grant this modality a place amongst the recognized chemotherapeutic agents, it is critical that we collect as much biological data as possible, given the possibility of this agent to be a meaningful adjuvant therapy.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments are described in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

In some embodiments, the present disclosure provides addressing the cancer patient through the steps of initially performing multi-platform phenotypic/molecular diagnostics (via Live-Cell Tumor Profiling—a laboratory technique to measure how cancer cells respond to a variety of drugs and drug combinations—or Liquid Biopsy ala NGS genomics, transcriptomics), followed by metabolomic profiling (via state-of-the-art Quantitative Mass Spectrometer). In some embodiments a living, or artificial intelligence driven Molecular Tumor Board is assembled and provides oversight which may include initiating cutting-edge cancer treatment protocols that apply biomarker-driven, and in-depth interrogation of tumor biology to craft unique patient-specific treatment regimens such as initial Total Neoadjuvant Therapy followed with complete post Adjuvant therapeutics. In some embodiments Short-course Targeted Radiation (includes very small doses of concomitant chemo-sensitizing agent to significantly increase overall biological efficacy at fewer than 10 fractions or 37 Gy maximum thus avoiding any significant collateral damage to surrounding tissue or organs). Furthermore, said patient may receive CT Guided Intratumoral Immunotherapy. As part of the treatment protocol, off-label use of medications to boost overall immunotherapy (medications used for other conditions can be repurposed to enhance the immune response) may be performed. Additionally, the patients will have issues resolved regarding leaky gut syndrome (enhance microbiome to achieve maximum immunotherapy potential). Other interventions include Hypoxia Tumor Targeting to address hypoxic tumors to generate global control of immunosuppression, treatment resistance, tumor growth, and increased metastasis. In other embodiments, Increasing immunogenicity of the Cancer Immune Microenvironment is increased since systemic immunotherapy relies on immune cells effectively infiltrating cancer cells (“immunologically hot”). To achieve maximum treatment benefit, tumors may be “heated up” or “primed”—which involves injecting specific immune agents directly into the tumor to attract immune cells to cancer. In other embodiments, advanced testing is performed to predict/target immunotherapeutic escape mechanisms; specific immunotherapies upregulate receptors to allow for escape and/or perhaps immunotherapeutic targets which means some combination of agents that work well in some cases, can still adjust and adapt therapy. Additional means include the utilization of Thermal Tumor Ablation (aka complete tumor destruction) a minimally invasive technique commonly used in the destruction of solid cancer tumors replacing the need for surgical removal of solid tumors. During tumor ablation, thermal energy is used to heat or cool tissue to cytotoxic levels. Further patient treatments include the utilization of Regenerative and Restorative Medicine (replace, restore, or revitalize cellular tissues or organs, which have been compromised and/or damaged by disease, trauma, or congenital symptoms), as well as Nutrition and Lifestyle Guidance (via blood-derived mass spectrometry ala individual patient metabolomic signatures which is the comprehensive analysis of metabolites in a biological specimen—an emerging technology that holds promise to inform the practice of precision medicine.)

In some embodiments, the method includes administering an antigen-nonspecific stimulatory means. Any antigen-nonspecific stimulatory means may be used, for example, Poly IC, BCG, cytokines such as interferon alpha and interleukin-2, or any adjuvant described herein.

In some embodiments, the method involves chemotherapy. Any chemotherapy may be used, for example alkylating agents (including nitrosoureas) such as altretamine, bendamustine, busulfan, carboplatin, chlorambucil, cisplatin, cyclophosphamide, dacarbazine, ifosfamide, mechlorethamine, melphalan, oxaliplatin, procarbazine, temozolomide, thiotepa, trabectedin, carmustine, lomustine, streptozocin; antimetabolites such as 5-fluorouracil, 6-mercaptopurine, azacitidine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, trifluridine/tipiracil combination; topoisomerase inhibitors such as etoposide, irinotecan, irinotecan liposomal, mitoxantrone, teniposide, topotecan; mitotic inhibitors (plant alkaloids) such as cabazitaxel, docetaxel, nab-paclitaxel, paclitaxel, vinblastine, vincristine, vincristine liposomal, vinorelbine; antitumor antibiotics (including anthracyclines) such as daunorubicin, doxorubicin, doxorubicin liposomal, epirubicin, idarubicin, mitoxantrone, valrubicin, bleomycin, dactinomycin, mitomycin-c; or other chemotherapy drugs such as all-trans-retinoic acid, arsenic trioxide, asparaginase, eribulin, ixabepilone, mitotane, omacetaxine, pegaspargase, procarbazine, romidepsin, ad vorinostat.

In some embodiments, the method includes administering low dose chemotherapy. “Low dose” refers to a treatment that is lower than traditional full or high-dose chemotherapy. For example, it may be between 5% and 50%, between 10% and 30% of the dosage of traditional chemotherapy such as the maximum tolerated dose (MTD).

### Predicting Response to Cancer Therapies

The present disclosure provides for assessment of cancer therapeutics in vitro prior to administration of said therapy in vivo. The in vitro assay is used to determine which therapy will be utilized in vivo. In some embodiments cancer cells are grown under conditions to expand cancer stem cells. One way of performing this is to isolate cancer stem cells using markers such as CD133. Isolation can be performed using various means known in the art including flow cytometry sorting, magnetic activated cell sorting, cellular panning, and various affinity means. In some embodiments cancer stem cells are expanded through the culture on feeder layers. Some of the feeder layers that are useful for the present disclosure are mesenchymal stem cells. In some embodiments murine embryonic fibroblasts are used as feeder cells.

In some embodiments, fibroblasts are transformed into cancer cells, or cancer-like cells through transfection with oncogenes and then exposed to exosomes from the cancer patients. In some embodiments, fibroblasts are transfected with oncogenes, said oncogenes can be selected from a group comprising of: ABCB1, ABCG2, ABI1, ABL1, ABL2, ACKR3, ACSL3, ACSL6, ACVR1B, ACVR2A, AFF1, AFF3, AFF4, AKAP9, AKT1, AKT2, AKT3, ALDH1A1, ALDH2, ALK, AMER1, ANGPT1, ANGPT2, ANKRD23, APC, AR, ARAF, AREG, ARFRP1, ARHGAP26, ARHGEF12, ARID1A, ARID1B, ARID2, ARNT, ASPSCR1, ASXL1, ATF1, ATIC, ATM, ATP1A1, ATP2B3, ATR, ATRX, AURKA, AURKB, AXIN1, AXL, BAP1, BARD1, BBC3, BCL10, BCL11A, BCL11B, BCL2, BCL2L1, BCL2L11, BCL2L2, BCL3, BCL6, BCL7A, BCL9, BCOR, BCORL1, BCR, BIRC3, BLM, BMPR1A, BRAF, BRCA1, BRCA2, BRD3, BRD4, BRINP3, BRIP1, BTG1, BTG2, BTK, BUB1B, C11orf30, C15orf65, C2orf44, CA6, CACNA1D, CALR, CAMTA1, CANT1, CARD11, CARS, CASC5, CASP8, CBFA2T3, CBFB, CBL, CBLB, CBLC, CCDC6, CCNB1IP1, CCND1, CCND2, CCND3, CCNE1, CD19, CD22, CD274, CD38, CD4, CD70, CD74, CD79A, CD79B, CD83, CDC73, CDH1, CDH11, CDK12, CDK4, CDK6, CDK7, CDK8, CDK9, CDKN1A, CDKN1B, CDKN2A, CDKN2B, CDKN2C, CDX2, CEBPA, CHCHD7, CHD2, CHD4, CHEK1, CHEK2, CHIC2, CHN1, CHORDC1, CIC, CIITA, CLP1, CLTC, CLTCL1, CNBP, CNOT3, CNTRL, COL1A1, COPB1, COX6C, CRBN, CREB1, CREB3L1, CREB3L2, CREBBP, CRKL, CRLF2, CRTC1, CRTC3, CSF1R, CSF3R, CTCF, CTLA4, CTNNA1, CTNNB1, CUL3, CXCR4, CYLD, CYP17A1, CYP2D6, DAXX, DDB2, DDIT3, DDR1, DDR2, DDX10, DDX3X, DDX5, DDX6, DEK, DICER1, DIS3, DLL4, DNM2, DNMT1, DNMT3A, DOT1L, DPYD, DUSP4, DUSP6, EBF1, ECT2L, EDNRB, EED, EGFR, EIF4A2, ELF4, ELK4, ELL, ELN, EMLA, EP300, EPHA3, EPHA5, EPHA7, EPHA8, EPHB1, EPHB2, EPHB4, EPS15, ERBB2, ERBB3, ERBB4, ERC1, ERCC1, ERCC2, ERCC3, ERCC4, ERCC5, EREG, ERG, ERN1, ERRFI1, ESR1, ETV1, ETV4, ETV5, ETV6, EWSR1, EXT1, EXT2, EZH2, EZR, FAF1, FAIM3, FAM46C, FANCA, FANCC, FANCD2, FANCE, FANCF, FANCG, FANCL, FAS, FAT1, FBXO11, FBXW7, FCRL4, FEV, FGF10, FGF14, FGF19, FGF2, FGF23, FGF3, FGF4, FGF6, FGFR1, FGFR1OP, FGFR2, FGFR3, FGFR4, FH, FHIT, FIP1L1, FKBP1A, FLCN, FLI1, FLT1, FLT3, FLT4, FNBP1, FOXA1, FOXL2, FOXO1, FOXO3, FOXO4, FOXP1, FRS2, FSTL3, FUBP1, FUS, GABRA6, GAS7, GATA1, GATA2, GATA3, GATA4, GATA6, GID4, GLI1, GMPS, GNA11, GNA12, GNA13, GNAQ, GNAS, GNRH1, GOLGA5, GOPC, GPC3, GPHN, GPR124, GRIN2A, GRM3, GSK3B, GUCY2C, H3F3A, H3F3B, HCK, HDAC1, HERPUD1, HEY1, HGF, HIP1, HIST1H1E, HIST1H3B, HIST1H4I, HLF, HMGA1, HMGA2, HMGN2P46, HNF1A, HNMT, HNRNPA2B1, HNRNPK, HOOK3, HOXA1I, HOXA13, HOXA9, HOXC11, HOXC13, HOXD11, HOXD13, HRAS, HSD3B1, HSP90AA1, HSP90AB1, IAPP, ID3, IDH1, IDH2, IGF1R, IGF2, IKBKE, IKZF1, IL2, IL21R, IL3RA, IL6, IL6ST, IL7R, INHBA, INPP4B, IRF2, IRF4, IRS2, ITGAV, ITGB1, ITK, ITPKB, JAK1, JAK2, JAK3, JAZF1, JUN, KAT6A, KAT6B, KCNJ5, KDM1A, KDM5A, KDM5C, KDM6A, KDR, KDSR, KEAP1, KEL, KIAA1549, KIF5B, KIR3DL1, KIT, KLF4, KLHL6, KLK2, KMT2A, KMT2C, KMT2D, KRAS, KTN1, LASP1, LCK, LCP1, LGALS3, LGR5, LHFP, LIFR, LMO1, LMO2, LOXL2, LPP, LRIG3, LRP1B, LUC7L2, LYL1, LYN, LZTR1, MAF, MAFB, MAGED1, MAGI2, MALT1, MAML2, MAP2K1, MAP2K2, MAP2K4, MAP3K1, MAPK1, MAPK11, MAX, MCL1, MDM2, MDM4, MDS2, MECOM, MED12, MEF2B, MEN1, MET, MITF, MKI67, MKL1, MLF1, MLH1, MLLT1, MLLT10, MLLT11, MLLT3, MLLT4, MLLT6, MMP9, MN1, MNX1, MPL, MRE11A, MS4A1, MSH2, MSH6, MSI2, MSN, MST1R, MTCP1, MTF2, MTOR, MUC1, MUC16, MUTYH, MYB, MYC, MYCL, MYCN, MYD88, MYH11, MYH9, NACA, NAE1, NBN, NCKIPSD, NCOA1, NCOA2, NCOA4, NDRG1, NF1, NF2, NFE2L2, NFIB, NFKB2, NFKB1A, NIN, NKX2-1, NONO, NOTCH1, NOTCH2, NOTCH3, NPM1, NR4A3, NRAS, NSD1, NT5C2, NTRK1, NTRK2, NTRK3, NUMA1, NUP214, NUP93, NUP98, NUTM1, NUTM2B, OLIG2, OMD, P2RY8, PAFAH1B2, PAK3, PALB2, PARK2, PARP1, PATZ1, PAX3, PAX5, PAX7, PAX8, PBRM1, PBX1, PCM1, PCSK7, PDCD1, PDCD1LG2, PDE4DIP, PDGFB, PDGFRA, PDGFRB, PDK1, PECAM1, PER1, PHF6, PHOX2B, PICALM, PIK3C2B, PIK3CA, PIK3CB, PIK3CD, PIK3CG, PIK3R1, PIK3R2, PIM1, PLAG1, PLCG2, PML, PMS1, PMS2, POLD1, POLE, POT1, POU2AF1, POU5F1, PPARG, PPP2R1A, PRCC, PRDM1, PRDM16, PREX2, PRF1, PRKAR1A, PRKC1, PRKDC, PRLR, PRPF40B, PRRT2, PRRX1, PRSS8, PSIP1, PSMD4, PTBP1, PTCH1, PTEN, PTK2, PTPN11, PTPRC, PTPRD, QKI, RABEP1, RAC1, RAD21, RAD50, RAD51, RAD51B, RAD51C, RAD51D, RAF1, RALGDS, RANBP17, RANBP2, RAP1GDS1, RARA, R131, RBM10, RBM15, RCOR1, RECQL4, REL, RELN, RET, RHOA, RHOH, RICTOR, RIPK1, RMI2, RNF213, RNF43, ROS1, RPL10, RPL22, RPL5, RPN1, RPS6KB1, RPTOR, RUNX1, RUNX1T1, S1PR2, SAMHD1, SBDS, SDC4, SDHA, SDHAF2, SDHB, SDHC, SDHD, SEPT5, SEPT6, SEPT9, SET, SETBP1, SETD2, SF1, SF3A1, SF3B1, SF3B2, SFPQ, SGK1, SH2B3, SH3GL1, SLAMF7, SLC34A2, SLC45A3, SLIT2, SMAD2, SMAD3, SMAD4, SMARCA4, SMARCB1, SMARCE1, SMC1A, SMC3, SMO, SNCA1P, SNX29, SOCS1, SOX10, SOX11, SOX2, SOX9, SPECC1, SPEN, SPOP, SPTA1, SRC, SRGAP3, SRSF2, SRSF3, SS18, SS18L1, SSX1, STAG2, STAT3, STAT4, STAT5B, STEAP1, STIL, STK11, SUFU, SUZ12, SYK, TAF1, TAF15, TAL1, TAL2, TBL1XR1, TBX3, TCEA1, TCF12, TCF3, TCF7L2, TCL1A, TEK, TERC, TERT, TET1, TET2, TFE3, TFEB, TFG, TFPT, TFRC, TGFB1, TGFBR2, THRAP3, TIMP1, TJP1, TLX1, TLX3, TM7SF2, TMPRSS2, TNFAIP3, TNFRSF14, TNFRSF17, TNFRSF18, TNFRSF9, TNFSF11, TOP1, TOP2A, TP53, TP63, TPBG, TPM3, TPM4, TPR, TRAF2, TRAF3, TRAF3IP3, TRAF7, TRIM26, TRIM27, TRIM33, TRIP11, TRRAP, TSC1, TSC2, TSHR, TTK, TTL, TYMS, U2AF1, U2AF2, UBA1, UBR5, USP6, VEGFA, VEGFB, VHL, VPS51, VTI1A, WAS, WEE1, WHSC1, WHSC1L1, WIF1, WISP3, WNT11, WNT2B, WNT3, WNT3A, WNT4, WNT5A, WNT6, WNT7B, WRN, WT1, WWTR1, XBP1, XPA, XPC, XPO1, YWHAE, YWHAZ, ZAK, ZBTB16, ZBTB2, ZMYM2, ZMYM3, ZNF217, ZNF331, ZNF384, ZNF521, ZNF703 and ZRSR2.

In some embodiments, cancer cells are grown under conditions of stem cell supernatant, said supernatant can be isolated from numerous types of regenerative cells. For generation of regenerative cells, the description below is provided. In some embodiments, cancer cells are cultured under hypoxia. In some embodiments, cancer cells are cultured in order to induce and/or augment expression of chemokine receptors. One such receptor is CXCR-4. The population of cells, including population of umbilical cord mesenchymal cells, may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition or alternatively, <1%, <2%, <3%, <4%, <5%, <6%, <7%, <8%, <9%, or <10% of the population of cells may express CD14 and/or CD45. Umbilical cord cells may further possess markers selected from the group consisting of STRO-1, CD105, CD54, CD56, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof. In some embodiments said placental cells are admixed with endothelial cells. Said endothelial cells may express one or more markers selected from the group consisting of: a) extracellular vimentin; b) CD133; c) c-kit; d) VEGF receptor; e) activated protein C receptor; and f) a combination thereof. In some embodiments, the population of endothelial cells comprises endothelial progenitor cells.

The population of cells may be allogeneic, autologous, or xenogenic to an individual, including an individual being administered the population of cells. In some embodiments, the population of cells are matched by mixed lymphocyte reaction matching.

In some embodiments, the population of cells is derived from tissue selected from the group consisting of the placental body, placenta, umbilical cord tissue, peripheral blood, hair follicle, cord blood, Wharton's Jelly, menstrual blood, endometrium, skin, omentum, amniotic fluid, and a combination thereof. In some embodiments, the population of cells, the population of umbilical mesenchymal stem cells, or the population of endothelial cells comprises human umbilical cord derived adherent cells. The human umbilical cord derived adherent cells may express a cytokines selected from the group consisting of) FGF-1; b) FGF-2; c) HGF; d) interleukin-1 receptor antagonist; and e) a combination thereof. In some embodiments, the population of cells, the population of umbilical cord cells express arginase, indoleamine 2,3 deoxygenase, interleukin-10, and/or interleukin 35. In some embodiments, the population of cells, the population of umbilical cord cells, or the population of endothelial cells express hTERT and Oct-4 but does not express a STRO-1 marker. In some embodiments, the population of cells, the population of umbilical cord cells has an ability to undergo cell division in less than 36 hours in a growth medium. In some embodiments, the population of cells, the population of umbilical cord cells has an ability to proliferate at a rate of 0.9-1.2 doublings per 36 hours in growth media. In some embodiments, the population of cells, the population of umbilical cord cells has an ability to proliferate at a rate of 0.9, 1.0, 1.1, or 1.2 doublings per 36 hours in growth media. The population of cells, population of umbilical cord cells may produce exosomes capable of inducing more than 50% proliferation when the exosomes are cultured with human umbilical cord endothelial cells. The induction of proliferation may occur when the exosomes are cultured with the human umbilical cord endothelial cells at a concentration of 10, 20, 30, 40, 50, 60, 70, 80, 90, 100, 110, 120, 130, 140, 150, 160, 170, 180, 190, 200, or more exosomes per cell.

### Alternatives

Embodiments of the present disclosure provided herein are described by way of the following numbered alternatives:

1. A method of treating cancer comprising the steps of: a) assessing a cancer patient for sensitivity of said cancer to a variety of therapeutic approaches; b) reducing tumor hypoxia and/or tumor acidosis in said cancer; c) altering the tumor microenvironment of said cancer; d) administering said cancer therapeutic approaches; e) optionally administering an antigen specific immune stimulatory means; and f) optionally administering an antigen-nonspecific stimulatory means.

2. The method of alternative 1, wherein said assessment of cancer patient for sensitivity to therapeutic approaches is ex vivo assessment of cancer samples for sensitivity to chemotherapy.

3. The method of alternative 2, wherein said assessment to sensitivity to chemotherapy is assessed by death of said cancer cells.

4. The method of alternative 3, wherein said death is assessed by mitochondrial depolarization.

5. The method of alternative 3, wherein said death is assessed by increased membrane permeability to molecules which normally reside outside of said membrane.

6. The method of alternative 5, wherein said death is assessed by trypan blue exclusion.

7. The method of alternative 3, wherein said cell death is apoptosis.

8. The method of alternative 3, wherein said cell death is necrosis.

9. The method of alternative 3, wherein said cell death is immunogenic cell death.

10. The method of alternative 9, wherein said immunogenic cell death is ferroptosis.

11. The method of alternative 9, wherein said immunogenic cell death is pyroptosis.

12. The method of alternative 9, wherein said immunogenic cell death is necroptosis.

13. The method of alternative 1, wherein said assessment of cancer patient for sensitivity to therapeutic approaches is ex vivo assessment of cancer samples for sensitivity to immunotherapy.

14. The method of alternative 1, wherein said assessment of cancer patient for sensitivity to therapeutic approaches is ex vivo assessment of cancer samples for sensitivity to metabolic therapy.

15. The method of alternative 1, wherein said assessment of cancer patient for sensitivity to therapeutic approaches is ex vivo assessment of cancer samples for sensitivity to nucleic acid therapy.

16. The method of alternative 1, wherein said assessment of cancer patient for sensitivity to therapeutic approaches is ex vivo assessment of cancer samples for sensitivity to electromagnetic therapy.

17. The method of alternative 1, wherein said assessment of cancer patient for sensitivity to therapeutic approaches is ex vivo assessment of cancer samples for sensitivity to radiation therapy.

18. The method of alternative 1, wherein said assessment of cancer patient for sensitivity to therapeutic approaches is ex vivo assessment of cancer samples for sensitivity to cryotherapy.

19. The method of alternative 1, wherein said assessment of cancer patient for sensitivity to therapeutic approaches is ex vivo assessment of cancer samples for sensitivity to hyperthermia.

20. The method of alternative 1, wherein said assessment of cancer patient for sensitivity to therapeutic approaches is ex vivo assessment of cancer stem cell samples for sensitivity to treatment.

21. The method of alternative 20, wherein said cancer stem cell expresses CD34.

22. The method of alternative 20, wherein said cancer stem cell expresses CD133.

23. The method of alternative 20, wherein said cancer stem cell expresses c-kit.

24. The method of alternative 20, wherein said cancer stem cell expresses aldehyde dehydrogenase.

25. The method of alternative 20, wherein said cancer stem cell expresses ABC transporters.

26. The method of alternative 20, wherein said cancer stem cell expresses CD47.

27. The method of alternative 20, wherein said cancer stem cell expresses DAF.

28. The method of alternative 1, wherein said reduction of hypoxia is achieved by administration of hyperbaric oxygen.

29. The method of alternative 1, wherein said reduction of hypoxia is achieved by administration of carbogen.

30. The method of alternative 1, wherein said reduction of hypoxia is achieved by administration of ozone therapy.

31. The method of alternative 1, wherein said reduction of hypoxia is reduction of effects of hypoxia.

32. The method of alternative 31, wherein said reduction of effects of hypoxia is reduction of hypoxia inducible factor-1 alpha (HIF-1 alpha).

33. The method of alternative 32, wherein said inhibition of HIF-1 alpha is accomplished by use of hammerhead ribozymes.

34. The method of alternative 32, wherein said inhibition of HIF-1 alpha is accomplished by use of decoy oligonucleotides.

35. The method of alternative 32, wherein said inhibition of HIF-1 alpha is accomplished by use of decoy peptides.

36. The method of alternative 32, wherein said inhibition of HIF-1 alpha is accomplished by use of antisense oligonucleotides.

37. The method of alternative 32, wherein said inhibition of HIF-1 alpha is accomplished by use of intracellularly delivered antibodies.

38. The method of alternative 32, wherein said inhibition of HIF-1 alpha is accomplished by use of intracellularly delivered microbodies.

39. The method of alternative 32, wherein said inhibition of HIF-1 alpha is accomplished by use of inducers of RNA Interference.

40. The method of alternative 32, wherein said inhibition of HIF-1 alpha is accomplished by use of siRNA.

41. The method of alternative 32, wherein said inhibition of HIF-1 alpha is accomplished by use of shRNA.

42. The method of alternative 32, wherein said inhibition of HIF-1 alpha is accomplished by administration of ascorbic acid.

43. The method of alternative 32, wherein said inhibition of HIF-1 alpha is accomplished by administration of CAY10585.

44. The method of alternative 32, wherein said inhibition of HIF-1 alpha is accomplished by administration of Chetomin.

45. The method of alternative 32, wherein said inhibition of HIF-1 alpha is accomplished by administration of Chrysin.

46. The method of alternative 32, wherein said inhibition of HIF-1 alpha is accomplished by administration of Dimethyloxaloylglycine (DMOG).

47. The method of alternative 32, wherein said inhibition of HIF-1 alpha is accomplished by administration of Echinomycin.

48. The method of alternative 32, wherein said inhibition of HIF-1 alpha is accomplished by administration of PX 12.

49. The method of alternative 32, wherein said inhibition of HIF-1 alpha is accomplished by administration of Vitexin.

50. The method of alternative 32, wherein said inhibition of HIF-1 alpha is accomplished by administration of YC-1.

51. The method of alternative 32, wherein said inhibition of HIF-1 alpha is accomplished by administration of an anthracycline.

52. The method of alternative 32, wherein said inhibition of HIF-1 alpha is accomplished by administration of an anthracycline prodrug.

53. The method of alternative 32, wherein said inhibition of HIF-1 alpha is accomplished by administration of rapamycin.

54. The method of alternative 32, wherein said inhibition of HIF-1 alpha is accomplished by administration of temsirolimus.

55. The method of alternative 32, wherein said inhibition of HIF-1 alpha is accomplished by administration of Everolimus.

56. The method of alternative 32, wherein said inhibition of HIF-1 alpha is accomplished by administration of geldanamycin.

57. The method of alternative 32, wherein said inhibition of HIF-1 alpha is accomplished by administration of 17-allylamino-17-demethoxy geldanamycin.

58. The method of alternative 32, wherein said inhibition of HIF-1 alpha is accomplished by administration of 17-dimethylaminoethylamino-17-demethoxy-geldanamycin.

59. The method of alternative 32, wherein said inhibition of HIF-1 alpha is accomplished by administration of quinocarmycin monocitrate.

60. The method of alternative 32, wherein said inhibition of HIF-1 alpha is accomplished by administration of hydrocyanization.

61. The method of alternative 32, wherein said inhibition of HIF-1 alpha is accomplished by administration of DX-52-1.

62. The method of alternative 32, wherein said inhibition of HIF-1 alpha is accomplished by administration of NSC-607097.

63. The method of alternative 32, wherein said inhibition of HIF-1 alpha is accomplished by administration of topotecan.

64. The method of alternative 32, wherein said inhibition of HIF-1 alpha is accomplished by administration of camptothecin,20-ester.

65. The method of alternative 32, wherein said inhibition of HIF-1 alpha is accomplished by administration of 9-glycineamido-20 (S).

66. The method of alternative 32, wherein said inhibition of HIF-1 alpha is accomplished by administration of paclitaxel.

67. The method of alternative 32, wherein said inhibition of HIF-1 alpha is accomplished by administration of docetaxel.

68. The method of alternative 32, wherein said inhibition of HIF-1 alpha is accomplished by administration of 2-methoxyestradiol.

69. The method of alternative 32, wherein said inhibition of HIF-1 alpha is accomplished by administration of vincristine.

70. The method of alternative 32, wherein said inhibition of HIF-1 alpha is accomplished by administration of discodermolide.

71. The method of alternative 32, wherein said inhibition of HIF-1 alpha is accomplished by administration of epothilone B.

72. The method of alternative 32, wherein said inhibition of HIF-1 alpha is accomplished by administration of Pleurotin.

73. The method of alternative 32, wherein said inhibition of HIF-1 alpha is accomplished by administration of wortmannin.

74. The method of alternative 32, wherein said inhibition of HIF-1 alpha is accomplished by administration of LY294002.

75. The method of alternative 32, wherein said inhibition of HIF-1 alpha is accomplished by administration of PD98059.

76. The method of alternative 32, wherein said inhibition of HIF-1 alpha is accomplished by administration of sodium butyrate.

77. The method of alternative 32, wherein said inhibition of HIF-1 alpha is accomplished by administration of sodium nitroprusside.

78. The method of alternative 32, wherein said inhibition of HIF-1 alpha is accomplished by administration of 103D5R.

79. The method of alternative 32, wherein said inhibition of HIF-1 alpha is accomplished by administration of S-2-amino3-[4V-N,N,-bis(2-chloroethyl)amino]-phenyl propionic acid N-oxide dihydrochloride.

80. The method of alternative 32, wherein said inhibition of HIF-1 alpha is accomplished by administration of depsipeptide.

81. The method of alternative 32, wherein said inhibition of HIF-1 alpha is accomplished by administration of genistein.

82. The method of alternative 32, wherein said inhibition of HIF-1 alpha is accomplished by administration of indanone.

83. The method of alternative 32, wherein said inhibition of HIF-1 alpha is accomplished by administration of staurosporin.

84. The method of alternative 32, wherein said inhibition of HIF-1 alpha is accomplished by administration of coumarins.

85. The method of alternative 32, wherein said inhibition of HIF-1 alpha is accomplished by administration of barbituric acid.

86. The method of alternative 32, wherein said inhibition of HIF-1 alpha is accomplished by administration of thiobarbituric acid.

87. The method of alternative 32, wherein said inhibition of HIF-1 alpha is accomplished by administration of aryloxyacetylamino enzoic acid.

88. The method of alternative 32, wherein said inhibition of HIF-1 alpha is accomplished by administration of digoxin.

89. The method of alternative 32, wherein said inhibition of HIF-1 alpha is accomplished by administration of hydroxamic acid.

90. The method of alternative 1, wherein tumor acidosis is treated by localized administration of bicarbonate.

91. The method of alternative 91, wherein said bicarbonate is administered by use of a carrier into said tumor microenvironment.

92. The method of alternative 91, wherein said bicarbonate is administered intratumorally with one or more agents selected from a group comprising of: a) doxorubicin; b) gemcitabine, cisplatin, methotrexate, 5-fluorouracil, betulinic acid, amphotericin B, diazepam, nystatin, propofol, testosterone, estrogen, prednisolone, prednisone, 2,3 mercaptopropanol, progesterone, docetaxel, or any combination thereof.

93. The method of alternative 92 wherein suppression of drug efflux is concurrently performed through administration of a p-glycoprotein inhibitor.

94. The method of alternative 93, wherein said p-glycoprotein inhibitor is verapamil.

95. The method of alternative 93, wherein said p-glycoprotein inhibitor is cyclosporin.

96. The method of alternative 93, wherein said p-glycoprotein inhibitor is tamoxifen.

97. The method of alternative 93, wherein said p-glycoprotein inhibitor is dexverapamil.

98. The method of alternative 93, wherein said p-glycoprotein inhibitor is dexniguldipine.

99. The method of alternative 93, wherein said p-glycoprotein inhibitor is valspodar.

100. The method of alternative 93, wherein said p-glycoprotein inhibitor is siRNA targeting one or more active domains of said p-glycoprotein.

101. The method of alternative 93, wherein said p-glycoprotein inhibitor is shRNA targeting one or more active domains of said p-glycoprotein.

102. The method of alternative 93, wherein said p-glycoprotein inhibitor is antisense oligonucleotides targeting one or more active domains of said p-glycoprotein.

103. The method of alternative 93, wherein said p-glycoprotein inhibitor is biricodar.

103. The method of alternative 93, wherein said p-glycoprotein inhibitor is tariquidar.

104. The method of alternative 93, wherein said p-glycoprotein inhibitor is zosuquidar.

105. The method of alternative 93, wherein said p-glycoprotein inhibitor is laniquidar.

106. The method of alternative 93, wherein said p-glycoprotein inhibitor is elacridar.

107. The method of alternative 93, wherein said p-glycoprotein inhibitor is timcodar.

108. The method of alternative 93, wherein said p-glycoprotein inhibitor is taxifolin.

109. The method of alternative 93, wherein said p-glycoprotein inhibitor is naringenin.

110. The method of alternative 93, wherein said p-glycoprotein inhibitor is diosmin.

111. The method of alternative 93, wherein said p-glycoprotein inhibitor is quercetin.

112. The method of alternative 93, wherein said p-glycoprotein inhibitor is diltiazem.

113. The method of alternative 93, wherein said p-glycoprotein inhibitor is bepridil.

114. The method of alternative 93, wherein said p-glycoprotein inhibitor is nicardipine.

115. The method of alternative 93, wherein said p-glycoprotein inhibitor is nifedipine.

116. The method of alternative 93, wherein said p-glycoprotein inhibitor is felodipine.

117. The method of alternative 93, wherein said p-glycoprotein inhibitor is isradipine.

118. The method of alternative 93, wherein said p-glycoprotein inhibitor is trifluoroperazine.

119. The method of alternative 93, wherein said p-glycoprotein inhibitor is clopenthixol.

120. The method of alternative 93, wherein said p-glycoprotein inhibitor is flupenthixol.

121. The method of alternative 93, wherein said p-glycoprotein inhibitor is emopamil.

122. The method of alternative 93, wherein said p-glycoprotein inhibitor is gallopamil.

123. The method of alternative 93, wherein said p-glycoprotein inhibitor is Ro11-2933.

124. The method of alternative 1, wherein said alteration of the tumor microenvironment is conversion of macrophage activity from M2 to M1.

125. The method of alternative 124, wherein said M2 are macrophages associated with pro-angiogenic activities.

126. The method of alternative 124, wherein said M1 are macrophages associated with anti-angiogenic activities.

127. The method of alternative 124, wherein said M2 are macrophages associated with arginase activity.

128. The method of alternative 124, wherein said M1 are macrophages associated with inducible nitric oxide synthase activity.

129. The method of alternative 124, wherein said M2 is converted to M1 by inhibition of arginase.

130. The method of alternative 129, wherein inhibition of arginase is accomplished by administration of a small molecule.

131. The method of alternative 130, wherein said small molecule is an arginine analogue.

132. The method of alternative 129, wherein said arginase is inhibited by suppression of mRNA encoding arginase.

133. The method of alternative 132, wherein suppression of arginase mRNA is accomplished by induction of RNA interference.

134. The method of alternative 133, wherein said RNA interference in induced by activation of DICER.

135. The method of alternative 133, wherein said RNA interference is induced by activation of argonaut.

136. The method of alternative 133, wherein said RNA interference is induced by administration of short interfering RNA.

137. The method of alternative 133, wherein said RNA interference is induced by administration of short hairpin RNA.

138. The method of alternative 124, wherein said conversion of M2 to M1 is accomplished by administration of an innate immune system activator.

139. The method of alternative 138, wherein said innate immune system activator is CpG containing DNA.

140. The method of alternative 138, wherein said innate immune system activator is a TLR9 activator.

141. The method of alternative 138, wherein said innate immune system activator is beta glucan.

142. The method of alternative 138, wherein said innate immune system activator is BCG.

143. The method of alternative 138, wherein said innate immune system activator is yeast wall extract.

144. The method of alternative 138, wherein said innate immune system activator is DNA extracellular traps.

145. The method of alternative 138, wherein said innate immune system activator is granzyme B.

146. The method of alternative 138, wherein said innate immune system activator is perforin.

147. The method of alternative 138, wherein said innate immune system activator is alpha galactosyl ceramide.

148. The method of alternative 138, wherein said innate immune system activator is TNF-alpha.

149. The method of alternative 138, wherein said innate immune system activator is an anti-idiotypic antibody to TNF-alpha.

150. The method of alternative 138, wherein said innate immune system activator is lymphotoxin.

151. The method of alternative 138, wherein said innate immune system activator is interferon gamma.

152. The method of alternative 151, wherein said interferon gamma is administered to the tumor microenvironment in a localized manner.

153. The method of alternative 152, wherein said interferon gamma is administered as a targeted nanoparticle.

154. The method of alternative 152, wherein said interferon gamma is administered as a liposome.

155. The method of alternative 152, wherein said interferon gamma is administered as an immunoliposome.

156. The method of alternative 155, wherein said immunoliposome is targeted to the tumor by means of affinity towards tumor associated surface antigens.

157. The method of alternative 156, wherein said tumor associated surface antigen is PSMA.

158. The method of alternative 156, wherein said tumor associated surface antigen is glypican.

159. The method of alternative 156, wherein said tumor associated surface antigen is EGF receptor.

160. The method of alternative 156, wherein said tumor associated surface antigen is c-met.

161. The method of alternative 156, wherein said tumor associated surface antigen is interleukin-3 receptor.

162. The method of alternative 156, wherein said tumor associated surface antigen is c-ERB.

163. The method of alternative 156, wherein said tumor associated surface antigen is HER2.

164. The method of alternative 156, wherein said tumor associated surface antigen is vimentin.

165. The method of alternative 156, wherein said tumor associated surface antigen is latency associated protein.

166. The method of alternative 156, wherein said tumor associated surface antigen is TGF-beta receptor.

167. The method of alternative 156, wherein said tumor associated surface antigen is CD25.

168. The method of alternative 156, wherein said tumor associated surface antigen is MAGE.

169. The method of alternative 156, wherein said tumor associated surface antigen is GAGE.

170. The method of alternative 156, wherein said tumor associated surface antigen is RAGE.

171. The method of alternative 156, wherein said tumor associated surface antigen is LRP.

172. The method of alternative 156, wherein said tumor associated surface antigen is gp96.

173. The method of alternative 156, wherein said tumor associated surface antigen is hsp 20.

174. The method of alternative 156, wherein said tumor associated surface antigen is hsp 25.

175. The method of alternative 156, wherein said tumor associated surface antigen is hsp 65.

176. The method of alternative 156, wherein said tumor associated surface antigen is CD38.

177. The method of alternative 156, wherein said tumor associated surface antigen is Fas ligand.

178. The method of alternative 156, wherein said tumor associated surface antigen is GITR ligand.

179. The method of alternative 156, wherein said tumor associated surface antigen is TNF alpha receptor p55.

180. The method of alternative 156, wherein said tumor associated surface antigen is TNF alpha receptor p75.

181. The method of alternative 156, wherein said tumor associated surface antigen is HLA-G.

182. The method of alternative 156, wherein said tumor associated surface antigen is HLA-E.

183. The method of alternative 156, wherein said tumor associated surface antigen is TLR-4.

184. The method of alternative 156, wherein said tumor associated surface antigen is IgG.

185. The method of alternative 156, wherein said tumor associated surface antigen is TRAIL.

186. The method of alternative 156, wherein said tumor associated surface antigen is BlyS.

187. The method of alternative 156, wherein said tumor associated surface antigen is LIGHT.

188. The method of alternative 156, wherein said tumor associated surface antigen is TRANCE.

189. The method of alternative 156, wherein said tumor associated surface antigen is osteopontin receptor.

190. The method of alternative 156, wherein said tumor associated surface antigen is calreticulin.

191. The method of alternative 156, wherein said tumor associated surface antigen is Annexin-V.

192. The method of alternative 156, wherein said tumor associated surface antigen is phosphatidylserine.

193. The method of alternative 156, wherein said tumor associated surface antigen is interleukin-10 receptor.

194. The method of alternative 156, wherein said tumor associated surface antigen is interleukin-13 receptor.

195. The method of alternative 156, wherein said tumor associated surface antigen is interleukin-4 receptor.

196. The method of alternative 156, wherein said tumor associated surface antigen is VEGF receptor.

197. The method of alternative 156, wherein said tumor associated surface antigen is IGF receptor.

198. The method of alternative 156, wherein said tumor associated surface antigen is FGF-1 receptor.

199. The method of alternative 156, wherein said tumor associated surface antigen is interleukin-6 receptor.

200. The method of alternative 156, wherein said tumor associated surface antigen is FGF-2 receptor.

201. The method of alternative 156, wherein said tumor associated surface antigen MUC-1.

202. The method of alternative 155, wherein said immunoliposome is targeted to a tumor stem cell.

203. The method of alternative 202, wherein said targeting to said tumor stem cell is performed by inserting antibodies to a tumor stem cell associated surface protein into said immunoliposome.

204. The method of alternative 202, wherein said targeting to said tumor stem cell is performed by inserting microbodies to a tumor stem cell associated surface protein into said immunoliposome.

205. The method of alternative 202, wherein said targeting to said tumor stem cell is performed by inserting camelid antibodies to a tumor stem cell associated surface protein into said immunoliposome.

206. The method of alternative 202, wherein said targeting to said tumor stem cell is performed by inserting tissue factor scaffold antibody-like molecules to a tumor stem cell associated surface protein into said immunoliposome.

207. The method of alternative 202, wherein said targeting to said tumor stem cell is performed by inserting selachimorpha antibodies to a tumor stem cell associated surface protein into said immunoliposome.

208. The method of alternative 203-207, wherein said tumor stem cell associated antigen is c-kit.

209. The method of alternative 203-207, wherein said tumor stem cell associated antigen is CD133.

210. The method of alternative 203-207, wherein said tumor stem cell associated antigen is SOX-2.

211. The method of alternative 203-207, wherein said tumor stem cell associated antigen is p-glycoprotein.

212. The method of alternative 203-207, wherein said tumor stem cell associated antigen is extracellular vimentin.

213. The method of alternative 1, wherein altering the tumor microenvironment of said cancer is performed by administration of agents that increase immunogenicity of cells in the tumor microenvironment.

214. The method of alternative 213, wherein said immunogenicity tumor microenvironment is upregulation of HLA I antigens by the tumor and/or cells surrounding said tumor.

215. The method of alternative 214, wherein said cells surrounding said tumor are macrophages.

216. The method of alternative 214, wherein said cells surrounding said tumor are type 2 macrophages.

217. The method of alternative 214, wherein said macrophages produce at least 10 ng/million cells of interleukin-10 under basal conditions.

218. The method of alternative 214, wherein said macrophages produce at least 5 ng/million cells of interleukin-6 under basal conditions.

219. The method of alternative 214, wherein said macrophages produce at least 10 ng/million cells of interleukin-4 under basal conditions.

220. The method of alternative 214, wherein said macrophages produce at least 20 ng/million cells of interleukin-13 under basal conditions.

221. The method of alternative 214, wherein said macrophages produce at least 10 ng/million cells of VEGF under basal conditions.

222. The method of alternative 214, wherein said macrophages produce at least 5 ng/million cells of angiopoietin under basal conditions.

223. The method of alternative 214, wherein said macrophages produce at least 100 ng/million cells of MMP3 under basal conditions.

224. The method of alternative 214, wherein said macrophages produce at least 50 ng/million cells of MM9 under basal conditions.

225. The method of alternative 214, wherein said macrophages produce at least 10 ng/million cells of MMP-13 under basal conditions.

226. The method of alternative 214, wherein said macrophages produce at least 10 ng/million cells of HGF-1 under basal conditions.

227. The method of alternative 213, wherein said agents that increase immunogenicity of cells in the tumor microenvironment are activators of dendritic cells.

228. The method of alternative 227, wherein said dendritic cell expresses CD200.

229. The method of alternative 227, wherein said dendritic cell expresses CD11c.

230. The method of alternative 227, wherein said dendritic cell expresses CD40.

231. The method of alternative 227, wherein said dendritic cell expresses CD80.

232. The method of alternative 227, wherein said dendritic cell expresses CD86.

233. The method of alternative 227, wherein said dendritic cell expresses DEC205.

234. The method of alternative 227, wherein said dendritic cell expresses CD8.

235. The method of alternative 227, wherein said dendritic cell expresses perforin.

236. The method of alternative 227, wherein said dendritic cell expresses granzyme.

237. The method of alternative 227, wherein said dendritic cell expresses nucleoporin.

238. The method of alternative 227, wherein said activated dendritic cells possess more nuclear translocated NF-kappa B compared to unactivated dendritic cells.

239. The method of alternative 227, wherein said activated dendritic cells possess more TAP-1 compared to unactivated dendritic cells.

240. The method of alternative 227, wherein said activated dendritic cells possess more open chromatin compared to unactivated dendritic cells.

241. The method of alternative 227, wherein said activated dendritic cells possess more phosphorylated GSK-3 compared to unactivated dendritic cells.

242. The method of alternative 227, wherein said activated dendritic cells secrete more interleukin-1 compared to unactivated dendritic cells.

243. The method of alternative 227, wherein said activated dendritic cells secrete more interleukin-2 compared to unactivated dendritic cells.

244. The method of alternative 227, wherein said activated dendritic cells secrete more interleukin-6 compared to unactivated dendritic cells.

245. The method of alternative 227, wherein said activated dendritic cells secrete more interleukin-12 compared to unactivated dendritic cells.

246. The method of alternative 227, wherein said activated dendritic cells secrete more interleukin-15 compared to unactivated dendritic cells.

247. The method of alternative 227, wherein said activated dendritic cells secrete more interleukin-18 compared to unactivated dendritic cells.

248. The method of alternative 227, wherein said activated dendritic cells secrete more interleukin-23 compared to unactivated dendritic cells.

249. The method of alternative 227, wherein said activated dendritic cells secrete more interleukin-27 compared to unactivated dendritic cells.

250. The method of alternative 227, wherein said activated dendritic cells secrete more interleukin-33 compared to unactivated dendritic cells.

251. The method of alternative 227, wherein said activated dendritic cells secrete more TRANCE compared to unactivated dendritic cells.

252. The method of alternative 227, wherein said activated dendritic cells secrete more perforin compared to unactivated dendritic cells.

253. The method of alternative 227, wherein said activated dendritic cells secrete more granzyme B compared to unactivated dendritic cells.

254. The method of alternative 227, wherein said activated dendritic cells secrete more LL-37 compared to unactivated dendritic cells.

255. The method of alternative 227, wherein said activated dendritic cells secrete more interferon alpha compared to unactivated dendritic cells.

256. The method of alternative 227, wherein said activated dendritic cells secrete more interferon gamma compared to unactivated dendritic cells.

257. The method of alternative 227, wherein said activator of said dendritic cell is neutrophil extracellular traps.

258. The method of alternative 227, wherein said activator of said dendritic cell is allogeneic T cells.

259. The method of alternative 258, wherein said allogeneic T cells are administered intratumorally.

260. The method of alternative 259, wherein said administered allogeneic T cells are preactivated prior to intratumoral administration.

261. The method of alternative 260, wherein said activation of said allogeneic T cells is performed by culture in immune stimulatory cytokines.

262. The method of alternative 261, wherein said immune stimulatory cytokines are cytokines associated with homeostatic expansion.

263. The method of alternative 261, wherein said immune stimulatory cytokines are cytokines associated with acquisition of a memory phenotype.

264. The method of alternative 261, wherein said immune stimulatory cytokines are cytokines associated with T cell homing activity.

265. The method of alternative 261, wherein said immune stimulatory cytokines are cytokines associated with acquisition of T helper cell activity.

266. The method of alternative 261, wherein said immune stimulatory cytokines are cytokines associated with acquisition of T cytotoxic cell activity.

267. The method of alternative 260, wherein said T cells are preactivated by ligation of the T cell receptor.

268. The method of alternative 260, wherein said T cells are preactivated by ligation of CD3.

269. The method of alternative 260, wherein said T cells are preactivated by ligation of the T cell receptor, CD3 and CD28.

270. The method of alternative 260, wherein said T cells are preactivated by ligation of the T cell receptor, CD3, CD28 and LFA-1.

271. The method of alternative 260, wherein said T cells are preactivated by ligation of the T cell receptor, CD3, CD28 and ICOS.

272. The method of alternative 260, wherein said T cells are preactivated by ligation of the T cell receptor, CD3, CD28, LFA-1 and ICOS.

273. The method of alternative 260, wherein said T cells are preactivated by ligation of the T cell receptor, CD3, CD28, LFA-1 and IL-12 receptor.

274. The method of alternative 260, wherein said T cells are preactivated by ligation of the T cell receptor, CD3, CD28, LFA-1 and IL-15 receptor.

275. The method of alternative 260, wherein said T cells are preactivated by ligation of the T cell receptor, CD3, CD28, LFA-1 and IL-18 receptor.

276. The method of alternative 260, wherein said T cells are preactivated by treatment with a lectin.

277. The method of alternative 276, wherein said lectin is concanavalin-A.

278. The method of alternative 276, wherein said lectin is phytohemagglutinin.

279. The method of alternative 276, wherein said lectin is cyanavirin.

280. The method of alternative 276, wherein said lectin is pokeweed mitogen.

281. The method of alternative 260, wherein said T cells are autologous.

282. The method of alternative 260, wherein said T cells are xenogeneic.

283. The method of alternative 260, wherein said T cells a population of mononuclear cells obtained from peripheral blood.

284. The method of alternative 260, wherein said T cells a population of mononuclear cells obtained from cord blood.

285. The method of alternative 260, wherein said T cells a population of mononuclear cells obtained from menstrual blood.

286. The method of alternative 260, wherein said T cells a population of mononuclear cells obtained from mobilized peripheral blood.

287. The method of alternative 286, wherein said peripheral blood is mobilized by treatment of the patient with G-CSF.

288. The method of alternative 286, wherein said peripheral blood is mobilized by treatment of the patient with GM-CSF.

289. The method of alternative 286, wherein said peripheral blood is mobilized by treatment of the patient with M-CSF.

290. The method of alternative 286, wherein said peripheral blood is mobilized by treatment of the patient with FLT-3 ligand.

291. The method of alternative 286, wherein said peripheral blood is mobilized by treatment of the patient with thrombopoietin.

292. The method of alternative 286, wherein said peripheral blood is mobilized by treatment of the patient with Mozobil.

293. The method of alternative 286, wherein said peripheral blood is mobilized by treatment of the patient with hyperbaric oxygen.

294. The method of alternative 286, wherein said peripheral blood is mobilized by treatment of the patient with ozone therapy.

295. The method of alternative 294, wherein said ozone therapy is administered by providing the patient with an ozonated liquid.

296. The method of alternative 295, wherein said ozonated liquid is saline. 297. The method of alternative 295, wherein said ozonated liquid is plasma.

298. The method of alternative 295, wherein said ozonated liquid is serum.

299. The method of alternative 295, wherein said ozonated liquid is blood.

300. The method of alternative 295, wherein said ozonated liquid is hypertonic saline.

301. The method of alternative 260, wherein said T cells are CD4 and CD8 T cells.

302. The method of alternative 260, wherein said T cells are CD3 T cells. 303. The method of alternative 260, wherein said T cells express CD4.

304. The method of alternative 260, wherein said T cells express CD8.

305. The method of alternative 260, wherein said T cells express alpha beta TCR.

306. The method of alternative 260, wherein said T cells express gamma delta TCR.

307. The method of alternative 260, wherein said T cells are NKT cells.

308. The method of alternative 260, wherein said T cells are activated by preculture in a mixed lymphocytes reaction.

309. The method of alternative 308, wherein said mixed lymphocyte reaction is performed using T cells together with allogeneic antigen presenting cells.

310. The method of alternative 309, wherein said antigen presenting cells express HLA I.

311. The method of alternative 309, wherein said antigen presenting cells express HLA II.

312. The method of alternative 309, wherein said antigen presenting cells express TAP-1.

313. The method of alternative 309, wherein said antigen presenting cells express CD40.

314. The method of alternative 309, wherein said antigen presenting cells express CD80.

315. The method of alternative 309, wherein said antigen presenting cells express CD86.

316. The method of alternative 309, wherein said antigen presenting cells are dendritic cells.

317. The method of alternative 309, wherein said antigen presenting cells are B cells.

318. The method of alternative 309, wherein said antigen presenting cells are B-1 cells.

319. The method of alternative 309, wherein said antigen presenting cells are monocytes.

320. The method of alternative 309, wherein said antigen presenting cells are macrophages.

321. The method of alternative 309, wherein said antigen presenting cells are allogeneic T cells.

322. The method of alternative 309, wherein said mixed lymphocyte reaction is performed in the presence of interleukin-2.

323. The method of alternative 309, wherein said mixed lymphocyte reaction is performed in the presence of interleukin-7.

324. The method of alternative 309, wherein said mixed lymphocyte reaction is performed in the presence of interleukin-12.

325. The method of alternative 309, wherein said mixed lymphocyte reaction is performed in the presence of interleukin-15.

326. The method of alternative 309, wherein said mixed lymphocyte reaction is performed in the presence of interleukin-18.

327. The method of alternative 309, wherein said mixed lymphocyte reaction is performed in the presence of interleukin-21.

328. The method of alternative 309, wherein said mixed lymphocyte reaction is performed in the presence of interleukin-23.

329. The method of alternative 309, wherein said mixed lymphocyte reaction is performed in the presence of interleukin-7.

330. The method of alternative 309, wherein said mixed lymphocyte reaction is performed in the presence of anti-CD25 antibody.

331. The method of alternative 309, wherein said mixed lymphocyte reaction is performed in the presence of anti-CTLA4 antibody.

332. The method of alternative 309, wherein said mixed lymphocyte reaction is performed in the presence of anti-IL-10 antibody.

333. The method of alternative 309, wherein said mixed lymphocyte reaction is performed in the presence of anti-TGF-beta antibody.

334. The method of alternative 309, wherein said mixed lymphocyte reaction is performed in the presence of anti-GITR antibody.

335. The method of alternative 309, wherein said mixed lymphocyte reaction is performed in the presence of anti-HLA-G antibody.

336. The method of alternative 309, wherein said mixed lymphocyte reaction is performed in the presence of anti-IL-10 receptor antibody.

337. The method of alternative 309, wherein said mixed lymphocyte reaction is performed in the presence of anti-TGF beta receptor antibody.

338. The method of alternative 309, wherein said mixed lymphocyte reaction is performed in the presence of hypoxia.

339. The method of alternative 338, wherein said hypoxia constitutes an oxygen concentration of 0.01% to 4% volume by volume.

340. The method of alternative 338, wherein said hypoxia constitutes an oxygen concentration of 0.1% to 3% volume by volume.

341. The method of alternative 338, wherein said hypoxia constitutes an oxygen concentration of 0.5% to 3% volume by volume.

342. The method of alternative 309, wherein said mixed lymphocyte reaction is performed in the presence of a toll like receptor agonist,

343. The method of alternative 342, wherein said toll like receptor agonist is Pam3Cys.

344. The method of alternative 342, wherein said toll like receptor agonist is CFA.

345. The method of alternative 342, wherein said toll like receptor agonist is MALP2.

346. The method of alternative 342, wherein said toll like receptor agonist is Pam2Cys.

347. The method of alternative 342, wherein said toll like receptor agonist is FSL-1.

348. The method of alternative 342, wherein said toll like receptor agonist is Hib-OMPC.

349. The method of alternative 342, wherein said toll like receptor agonist is polyribosinic:polyribocytidic acid.

350. The method of alternative 342, wherein said toll like receptor agonist is polyadenosine-polyuridylic acid.

351. The method of alternative 342, wherein said toll like receptor agonist is Polyinosinic-Polycytidylic acid stabilized with poly-L-lysine and carboxymethylcellulose.

352. The method of alternative 342, wherein said toll like receptor agonist is monophosphoryl lipid A.

353. The method of alternative 342, wherein said toll like receptor agonist is HMGB1

354. The method of alternative 342, wherein said toll like receptor agonist is flagellin.

355. The method of alternative 342, wherein said toll like receptor agonist is free histones.

356. The method of alternative 342, wherein said toll like receptor agonist is low molecular weight hyaluronic acid.

357. The method of alternative 342, wherein said toll like receptor agonist is dendritic cell lysate.

358. The method of alternative 342, wherein said toll like receptor agonist is neutrophil lysate.

359. The method of alternative 342, wherein said toll like receptor agonist is lysates from monocytes exposed to inflammatory cytokines.

360. The method of alternative 350, wherein said monocytes are treated with 10 ng-100 ng of TNF-alpha per 1 million monocytes.

361. The method of alternative 350, wherein said monocytes are treated with 20 ng-80 ng of TNF-alpha per 1 million monocytes.

362. The method of alternative 350, wherein said monocytes are treated with 40 ng-50 ng of TNF-alpha per 1 million monocytes.

363. The method of alternative 350, wherein said monocytes are treated with 1 ng-500 ng of interferon-alpha per 1 million monocytes.

364. The method of alternative 350, wherein said monocytes are treated with 10 ng-100 ng of interferon-alpha per 1 million monocytes.

365. The method of alternative 350, wherein said monocytes are treated with 50 ng-250 ng of interferon-alpha per 1 million monocytes.

366. The method of alternative 350, wherein said monocytes are treated with 100 ng-200 ng of interferon-alpha per 1 million monocytes.

367. The method of alternative 350, wherein said monocytes are treated with 1 ng-500 ng of interferon-gamma per 1 million monocytes.

368. The method of alternative 350, wherein said monocytes are treated with 10 ng-250 ng of interferon-gamma per 1 million monocytes.

369. The method of alternative 350, wherein said monocytes are treated with 100 ng-250 ng of interferon-gamma per 1 million monocytes.

370. The method of alternative 350, wherein said monocytes are treated with 200-500 ng of interferon-gamma per 1 million monocytes.

371. The method of alternative 342, wherein said toll like receptor agonist is sialyl-Tn (STn).

372. The method of alternative 342, wherein said toll like receptor agonist is imiquimod.

373. The method of alternative 342, wherein said toll like receptor agonist is resiquimod.

374. The method of alternative 342, wherein said toll like receptor agonist is loxoribine.

375. The method of alternative 342, wherein said toll like receptor agonist is unmethylated CpG dinucleotide (CpG-ODN).

376. The method of alternative 1, wherein said alteration of said tumor microenvironment is accomplished by administration of regenerative cells transfected with one or more immunogenic agents.

377. The method of alternative 376, wherein said transfected immunogenic agent is under transcriptional control of an inducible promoter.

378. The method of alternative 377, wherein said promoter is activated subsequent to increase of plasma concentration of an inducing agent.

379. The method of alternative 377, wherein said immunogenic agent is a toll like receptor.

380. The method of alternative 377, wherein said immunogenic agent is a toll like receptor agonist.

381. The method of alternative 377, wherein said immunogenic agent is HMGB1.

382. The method of alternative 377, wherein said immunogenic agent is TNF-alpha.

383. The method of alternative 377, wherein said immunogenic agent is lymphotoxin.

384. The method of alternative 377, wherein said immunogenic agent is TRANCE.

385. The method of alternative 377, wherein said immunogenic agent is BlyS.

386. The method of alternative 377, wherein said immunogenic agent is interferon gamma.

387. The method of alternative 377, wherein said immunogenic agent is interferon alpha.

388. The method of alternative 377, wherein said immunogenic agent is interferon beta.

389. The method of alternative 377, wherein said immunogenic agent is interferon tau.

390. The method of alternative 377, wherein said immunogenic agent is interferon omega.

391. The method of alternative 377, wherein said immunogenic agent is interleukin-17.

392. The method of alternative 377, wherein said immunogenic agent is calreticulin.

393. The method of alternative 377, wherein said immunogenic agent is gp96.

394. The method of alternative 377, wherein said immunogenic agent is interleukin-2.

395. The method of alternative 377, wherein said immunogenic agent is interleukin-15.

396. The method of alternative 377, wherein said immunogenic agent is interleukin-18.

397. The method of alternative 377, wherein said immunogenic agent is alpha galactosylceramide.

398. The method of alternative 377, wherein said immunogenic agent is interleukin-21.

399. The method of alternative 377, wherein said immunogenic agent is interleukin-23.

400. The method of alternative 377, wherein said immunogenic agent is interleukin-33.

401. The method of alternative 376, wherein said regenerative cell is engineered to increase chemoattraction towards tumor cells.

402. The method of alternative 401, wherein said regenerative cell is engineered by transfection with CXCR4.

403. The method of alternative 401, wherein said regenerative cell is engineered by transfection with MIP-1 alpha.

404. The method of alternative 401, wherein said regenerative cell is engineered by transfection with MIP-1 beta.

405. The method of alternative 401, wherein said regenerative cell is engineered by transfection with interleukin-8 receptor.

406. The method of alternative 401, wherein said regenerative cell is engineered by culture in hypoxia.

407. The method of alternative 401, wherein said regenerative cell is engineered by culture in acidic conditions.

408. The method of alternative 401, wherein said regenerative cell is engineered by culture in TGF-beta.

409. The method of alternative 401, wherein said regenerative cell is engineered by culture in TNF-alpha.

410. The method of alternative 401, wherein said regenerative cell is engineered by culture with an activator of NF-kappa B.

411. The method of alternative 401, wherein said regenerative cell is engineered by culture with an inhibitor of I kappa B.

412. The method of alternative 401, wherein said regenerative cell is engineered by culture with an activator of SMAD2.

413. The method of alternative 401, wherein said regenerative cell is engineered by culture with an activator of SMAD4.

414. The method of alternative 401, wherein said regenerative cell is engineered by culture with an activator of latency activator protein.

415. The method of alternative 401, wherein said regenerative cell is engineered by culture with an activator of TNF-alpha receptor p55.

416. The method of alternative 401, wherein said regenerative cell is engineered by culture with an activator of TNF-alpha receptor p75.

417. The method of alternative 401, wherein said regenerative cell is engineered by culture with an activator of TNF-alpha receptor p75.

418. The method of alternative 401, wherein said regenerative cells are mesenchymal stem cells.

419. The method of alternative 418, wherein said mesenchymal cells are obtained from bone marrow.

420. The method of alternative 418, wherein said mesenchymal cells are obtained from umbilical cord blood, placenta or combinations thereof.

421. The method of alternative 418, wherein said regenerative cells are extracted from a tissue based on expression of CD73.

422. The method of alternative 421, wherein said tissue is bone marrow matrix tissue.

423. The method of alternative 422, wherein said regenerative cells are extracted from an area possessing hypoxia of said bone marrow tissue.

424. The method of alternative 422, wherein said umbilical cord tissue is perfused with media containing 1% v/v carbon monoxide in a saline solution prior to extraction of said cells.

425. The method of alternative 424, wherein said cells are plastic adherent.

426. The method of alternative 425, wherein said cells are mesenchymal stem cells.

427. The method of alternative 426, wherein said mesenchymal stem cells are pre-treated with a regenerative adjuvant prior to administration.

428. The method of alternative 427, wherein said regenerative adjuvant is platelet rich plasma.

429. The method of alternative 427, wherein said mesenchymal stem cells are treated with said platelet rich plasma for 1 minute to 1 week prior to administration.

430. The method of alternative 429, wherein said mesenchymal stem cells are incubated with said platelet rich plasma in the presence of DMEM media.

431. The method of alternative 430, wherein said mesenchymal stem cells are incubated with said platelet rich plasma in the presence of EMEM media.

432. The method of alternative 430, wherein said mesenchymal stem cells are incubated with said platelet rich plasma in the presence of RPMI-1640 media.

433. The method of alternative 432, wherein said mesenchymal stem cells are incubated with said platelet rich plasma in the presence of OPTI-MEM media.

434. The method of alternative 433, wherein said mesenchymal stem cells are incubated with said platelet rich plasma in the presence of a growth factor.

435. The method of alternative 434, wherein said growth factor is selected from a group comprising of; a) HGF-1; b) EGF-1; c) FGF-1; d) FGF-2 and e) hematopoietic stem cell conditioned media.

436. The method of alternative 435, wherein said hematopoietic stem cells possess interleukin-3 receptor.

437. The method of alternative 435, wherein said hematopoietic stem cells possess HGF-1 receptor.

438. The method of alternative 435, wherein said hematopoietic stem cells possess interleukin-6 receptor.

439. The method of alternative 435, wherein said hematopoietic stem cells possess interleukin-8 receptor.

440. The method of alternative 435, wherein said hematopoietic stem cells possess interleukin-10 receptor.

441. The method of alternative 435, wherein said hematopoietic stem cells possess interleukin-11 receptor.

442. The method of alternative 435, wherein said hematopoietic stem cells possess interleukin-20 receptor.

443. The method of alternative 435, wherein said hematopoietic stem cells possess thrombopoietin receptor.

444. The method of alternative 435, wherein said hematopoietic stem cells possess FGF-1 receptor.

445. The method of alternative 435, wherein said hematopoietic stem cells possess FGF-2 receptor.

446. The method of alternative 435, wherein said hematopoietic stem cells possess FGF-5 receptor.

447. The method of alternative 435, wherein said hematopoietic stem cells possess TGF-beta receptor.

448. The method of alternative 435, wherein said hematopoietic stem cells possess endoglin receptor.

449. The method of alternative 435, wherein said hematopoietic stem cells possess the ability to produce autocrine TGF-beta.

450. The method of alternative 435, wherein said hematopoietic stem cells possess leukemia inhibitory factor receptor.

452. The method of alternative 435 wherein said hematopoietic stem cells possess VEGF receptor.

453. The method of alternative 435, wherein said hematopoietic stem cells possess PGE2 receptor.

454. The method of alternative 435, wherein said hematopoietic stem cells possess interferon gamma receptor.

455. The method of alternative 435, wherein said hematopoietic stem cells express RANK ligand.

456. The method of alternative 435, wherein said hematopoietic stem cells possess CD34.

457. The method of alternative 435, wherein said hematopoietic stem cells possess CD133.

458. The method of alternative 435, wherein said hematopoietic stem cells possess CD34 and CD133.

459. The method of alternative 435, wherein said hematopoietic stem cells lack expression of CD38.

460. The method of alternative 435, wherein said hematopoietic stem cells possess interleukin-3 receptor.

461. The method of alternative 376, wherein said regenerative cells are CD56 expressing mesenchymal stem cells.

462. The method of alternative 461, wherein said mesenchymal stem cells are generated in vitro.

463. The method of alternative 462, wherein said naturally occurring mesenchymal stem cells are tissue derived.

464. The method of alternative 462, wherein said naturally occurring mesenchymal stem cells are derived from a bodily fluid.

465. The method of alternative 463, wherein said tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; 1) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

466. The method of alternative 463, wherein said tissue derived mesenchymal stem cells are isolated from tissues containing cells selected from a group of cells comprising of: endothelial cells, epithelial cells, d8rmal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. Bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, Macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of car), stria vascularis basal cells, stria vascularis marginal cell (lining endolymphatic space of car), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

466. The method of alternative 461, wherein said mesenchymal stem cells are plastic adherent.

467. The method of alternative 461, wherein said mesenchymal stem cells express a marker selected from a group comprising of: a) CD73; b) CD90; and c) CD105.

468. The method of alternative 467, wherein said mesenchymal stem cells lack expression of a marker selected from a group comprising of: a) CD14; b) CD45; and c) CD34.

469. The method of alternative 467, wherein said mesenchymal stem cells from umbilical cord tissue express markers selected from a group comprising of; a) oxidized low density lipoprotein receptor 1, b) chemokine receptor ligand 3; and c) granulocyte chemotactic protein.

470. The method of alternative 467, wherein said mesenchymal stem cells from umbilical cord tissue do not express markers selected from a group comprising of: a) CD117; b) CD31; c) CD34; and CD45;

471. The method of alternative 467, wherein said mesenchymal stem cells from umbilical cord tissue express, relative to a human fibroblast, increased levels of interleukin 8 and reticulon 1

472. The method of alternative 467, wherein said mesenchymal stem cells from umbilical cord tissue have the potential to differentiate into cells of at least a skeletal muscle, vascular smooth muscle, pericyte or vascular endothelium phenotype.

473. The method of alternative 467, wherein said mesenchymal stem cells from umbilical cord tissue express markers selected from a group comprising of: a) CD10; b) CD13; c) CD44; d) CD73; and e) CD90.

474. The method of alternative 467, wherein said umbilical cord tissue mesenchymal stem cell is an isolated umbilical cord tissue cell isolated from umbilical cord tissue substantially free of blood that is capable of self-renewal and expansion in culture,

475. The method of alternative 467, wherein said umbilical cord tissue mesenchymal stem cells has the potential to differentiate into cells of other phenotypes.

476. The method of alternative 475, wherein said other phenotypes comprise: a) osteocytic; b) adipogenic; and c) chondrogenic differentiation.

477. The method of alternative 475, wherein said cord tissue derived mesenchymal stem cells can undergo at least 20 doublings in culture.

478. The method of alternative 475, wherein said cord tissue derived mesenchymal stem cell maintains a normal karyotype upon passaging

479. The method of alternative 475, wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

480. The method of alternative 475, wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

481. The method of alternative 475, wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; 1) RANTES; and m) TIMP1

482. The method of alternative 475, wherein said umbilical cord tissue derived cells express markers selected from a group comprising of: a) TRA1-60; b) TRA1-81; c) SSEA3; d) SSEA4; and e) NANOG.

483. The method of alternative 475, wherein said umbilical cord tissue-derived cells are positive for alkaline phosphatase staining.

484. The method of alternative 475, wherein said umbilical cord tissue-derived cells are capable of differentiating into one or more lineages selected from a group comprising of; a) ectoderm; b) mesoderm, and; c) endoderm.

485. The method of alternative 475, wherein said bone marrow derived mesenchymal stem cells possess markers selected from a group comprising of: a) CD73; b) CD90; and c) CD105.

486. The method of alternative 475, wherein said bone marrow derived mesenchymal stem cells possess markers selected from a group comprising of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; 1) 6-19; m) thrombomodulin; n) telomerase; o) CD10; p) CD13; and q) integrin beta.

487. The method of alternative 475, wherein said bone marrow derived mesenchymal stem cell is a mesenchymal stem cell progenitor cell.

488. The method of alternative 487, wherein said mesenchymal progenitor cells are a population of bone marrow mesenchymal stem cells enriched for cells containing STRO-1

489. The method of alternative 488, wherein said mesenchymal progenitor cells express both STRO-1 and VCAM-1.

490. The method of alternative 488, wherein said STRO-1 expressing cells are negative for at least one marker selected from the group consisting of: a) CBFA-1; b) collagen type II; c) PPAR.gamma2; d) osteopontin; e) osteocalcin; f) parathyroid hormone receptor; g) leptin; h) H-ALBP; i) aggrecan; j) Ki67, and k) glycophorin A.

491. The method of alternative 488, wherein said bone marrow mesenchymal stem cells lack expression of CD14, CD34, and CD45.

492. The method of alternative 491, wherein said STRO-1 expressing cells are positive for a marker selected from a group comprising of: a) VACM-1; b) TKY-1; c) CD146 and; d) STRO-2

493. The method of alternative 488, wherein said bone marrow mesenchymal stem cell express markers selected from a group comprising of: a) CD13; b) CD34; c) CD56 and; d) CD117

494. The method of alternative 493, wherein said bone marrow mesenchymal stem cells do not express CD10.

495. The method of alternative 493, wherein said bone marrow mesenchymal stem cells do not express CD2, CD5, CD14, CD19, CD33, CD45, and DRII.

496. The method of alternative 493, wherein said bone marrow mesenchymal stem cells express CD13, CD34, CD56, CD90, CD117 and nestin, and which do not express CD2, CD3, CD10, CD14, CD16, CD31, CD33, CD45 and CD64.

497. The method of alternative 466, wherein said skeletal muscle stem cells express markers selected from a group comprising of: a) CD13; b) CD34; c) CD56 and; d) CD117

498. The method of alternative 497, wherein said skeletal muscle mesenchymal stem cells do not express CD10.

499. The method of alternative 497, wherein said skeletal muscle mesenchymal stem cells do not express CD2, CD5, CD14, CD19, CD33, CD45, and DRII.

500. The method of alternative 497, wherein said bone marrow mesenchymal stem cells express CD13, CD34, CD56, CD90, CD117 and nestin, and which do not express CD2, CD3, CD10, CD14, CD16, CD31, CD33, CD45 and CD64.

501. The method of alternative 376, wherein said immunogenic agent is a virus.

502. The method of alternative 501, wherein said virus possesses a selectivity towards neoplastic tissue.

503. The method of alternative 501, wherein said virus is transfected to increase immunogenicity.

504. The method of alternative 503, wherein said virus is transfected to express an immune stimulatory protein.

505. The method of alternative 504, wherein said immune stimulatory protein is interleukin-1 beta.

506. The method of alternative 504, wherein said immune stimulatory protein is interleukin-2.

507. The method of alternative 504, wherein said immune stimulatory protein is interleukin-4.

508. The method of alternative 504, wherein said immune stimulatory protein is interleukin-6.

509. The method of alternative 504, wherein said immune stimulatory protein is interleukin-10.

510. The method of alternative 504, wherein said immune stimulatory protein is interleukin-12.

511. The method of alternative 504, wherein said immune stimulatory protein is interleukin-15.

512. The method of alternative 504, wherein said immune stimulatory protein is interleukin-18.

513. The method of alternative 504, wherein said immune stimulatory protein is interleukin-21.

514. The method of alternative 504, wherein said immune stimulatory protein is interleukin-33.

515. The method of alternative 504, wherein said immune stimulatory protein is HMGB1.

516. The method of alternative 504, wherein said immune stimulatory protein is complement C1q.

517. The method of alternative 504, wherein said immune stimulatory protein is C3.

518. The method of alternative 504, wherein said immune stimulatory protein is C5.

519. The method of alternative 504, wherein said immune stimulatory protein is interleukin-6 complexed with soluble interleukin-6 receptor.

520. The method of alternative 504, wherein said immune stimulatory protein is HMGB1.

521. The method of alternative 504, wherein said immune stimulatory protein is GM-CSF.

522. The method of alternative 504, wherein said immune stimulatory protein is M-CSF.

523. The method of alternative 504, wherein said immune stimulatory protein is G-CSF.

524. The method of alternative 504, wherein said immune stimulatory protein is antibody to TGF-beta.

525. The method of alternative 504, wherein said immune stimulatory protein is antibody to IL-10.

526. The method of alternative 501, wherein said virus is a vaccinia virus.

527. The method of alternative 526, wherein said vaccinia virus is Lister strain.

528. The method of alternative 526, wherein said vaccinia virus is Western Reserve strain.

529. The method of alternative 526, wherein said vaccinia virus is Copenhagen strain.

530. The method of alternative 526, wherein said vaccinia virus is Bern strain.

531. The method of alternative 526, wherein said vaccinia virus is Paris strain.

532. The method of alternative 526, wherein said vaccinia virus is Tashkent strain.

533. The method of alternative 526, wherein said vaccinia virus is Tian Tan strain.

534. The method of alternative 526, wherein said vaccinia virus is Wyeth strain.

535. The method of alternative 526, wherein said vaccinia virus is IHD-J strain.

536. The method of alternative 526, wherein said vaccinia virus is IHD-W strain.

537. The method of alternative 526, wherein said vaccinia virus is Brighton strain.

538. The method of alternative 526, wherein said vaccinia virus is Ankara strain.

539. The method of alternative 526, wherein said vaccinia virus is CVA382 strain.

540. The method of alternative 526, wherein said vaccinia virus is Modified Vaccinia Ankara strain.

541. The method of alternative 526, wherein said vaccinia virus is Dairen I strain.

542. The method of alternative 526, wherein said vaccinia virus is LC16m8 strain.

543. The method of alternative 526, wherein said vaccinia virus is LC16M0 strain.

544. The method of alternative 526, wherein said vaccinia virus is LIVP strain.

545. The method of alternative 526, wherein said vaccinia virus is ACAM2000 strain.

546. The method of alternative 526, wherein said vaccinia virus is WR 65-16 strain.

547. The method of alternative 526, wherein said vaccinia virus is Connaught strain.

548. The method of alternative 526, wherein said vaccinia virus is NYCBH strain.

549. The method of alternative 526, wherein said vaccinia virus is NYVAC strain.

550. The method of alternative 501, wherein said virus is a reovirus.

551. The method of alternative 550, wherein said reovirus is Type 3 (Dearing) reovirus.

552. The method of alternative 550, wherein said reovirus is Type 1 (Lang) reovirus.

553. The method of alternative 550, wherein said reovirus is Type 2 (Jones) reovirus.

554. The method of alternative 550, wherein said reovirus is Type 3 (Abney) reovirus.

555. The method of alternative 501, wherein said virus is a herpes virus.

556. The method of alternative 555, wherein said herpes virus.

557. The method of alternative 501, wherein said oncolytic virus is administered together with an inhibitor of the complement cascade.

558. The method of alternative 557, wherein said inhibitor of the complement cascade is an agent that induces RNA interference to one or more members of the complement pathway.

559. The method of alternative 558, wherein said agent is a short interfering RNA molecule.

560. The method of alternative 558, wherein said agent is a short hairpin RNA (shRNA) molecule.

561. The method of alternative 560, wherein said shRNA is delivered by means of gene transfection.

562. The method of alternative 560, wherein said shRNA is delivered by means of hydrodynamic administration.

563. The method of alternative 560, wherein said shRNA is delivered by means of viral delivery.

564. The method of alternative 560, wherein said shRNA is delivered by means of an adenovirus.

565. The method of alternative 560, wherein said shRNA is delivered by means of a lentivirus.

566. The method of alternative 560, wherein said shRNA is delivered by means of a vaccinia virus.

567. The method of alternative 560, wherein said shRNA is delivered by means of an adenoassociated virus.

568. The method of alternative 560, wherein said shRNA is delivered by means of an adenovirus-5.

569. The method of alternative 560, wherein said shRNA is delivered by means of HSV-1.

570. The method of alternative 560, wherein said shRNA is delivered by means of HSV-2.

571. The method of alternative 569 and 570, wherein one or more virulence genes is deleted from said virus.

572. The method of alternative 571, wherein said virulence gene is gamma-34.5.

573. The method of alternative 571, wherein said virulence gene is ICP-47. 574. The method of alternative 571, wherein said virulence gene is ICP-6.

575. The method of alternative 571, wherein said virulence gene is US11.

576. The method of alternative 571, wherein said virulence gene is US3

577. The method of alternative 1, wherein said tumor microenvironment is modified by administration of a natural killer cell population.

578. The method of alternative 577, wherein said natural killer cell population is administered intratumorally.

579. The method of alternative 577, wherein said natural killer cell population is administered systemically.

580. The method of alternative 577, wherein said natural killer cell population is administered peritumorally.

581. The method of alternative 577, wherein said natural killer cell population expresses CD56.

582. The method of alternative 577, wherein said natural killer cell population expresses CD57.

583. The method of alternative 577, wherein said natural killer cell population expresses CD16.

584. The method of alternative 577, wherein said natural killer cell population expresses NKG2D.

585. The method of alternative 577, wherein said natural killer cell population expresses NCR1.

586. The method of alternative 577, wherein said natural killer cell population expresses NKG2A.

587. The method of alternative 577, wherein said natural killer cell population expresses NCR2.

588. The method of alternative 577, wherein said natural killer cell population expresses NCR3.

589. The method of alternative 577, wherein said natural killer cell population expresses CD226.

590. The method of alternative 577, wherein said natural killer cell population expresses CD224.

591. The method of alternative 577, wherein said natural killer cell population expresses NCAM.

592. The method of alternative 577, wherein said natural killer cell population expresses CD25.

593. The method of alternative 577, wherein said natural killer cell population expresses CD94.

594. The method of alternative 577, wherein said natural killer cell population expresses CD117.

595. The method of alternative 577, wherein said natural killer cell population expresses KLRC2.

596. The method of alternative 577, wherein said natural killer cell population expresses KLRF1.

597. The method of alternative 577, wherein said natural killer cell population expresses CD69.

598. The method of alternative 577, wherein said natural killer cell population expresses CD11b.

599. The method of alternative 577, wherein said natural killer cell population expresses KLRB1.

600. The method of alternative 577, wherein said natural killer cell population expresses CD122.

601. The method of alternative 577, wherein said natural killer cell population expresses EOMES.

602. The method of alternative 577, wherein said natural killer cell population expresses CD27.

603. The method of alternative 577, wherein said natural killer cell population expresses TBX21.

604. The method of alternative 577, wherein said natural killer cell population expresses KIR2DL1.

605. The method of alternative 577, wherein said natural killer cell population expresses CD54.

606. The method of alternative 577, wherein said natural killer cell population expresses perforin.

607. The method of alternative 577, wherein said natural killer cell population expresses granzyme B.

608. The method of alternative 577, wherein said natural killer cell is extracted from peripheral blood and amplified in vitro.

609. The method of alternative 608, wherein said amplification of said natural killer cell is expansion of numbers of said cells in vitro or in vivo.

610. The method of alternative 608, wherein said amplification of said natural killer cell is enhancement of cytotoxic activity of said cells in vitro or in vivo.

611. The method of alternative 608, wherein said amplification of said natural killer cells is accomplished by culture in a cytokine containing media.

612. The method of alternative 611, wherein said cytokine containing media contains interleukin-2.

613. The method of alternative 612, wherein said interleukin-2 is administered at a concentration sufficient to activate both the interleukin-2 alpha and beta receptors.

614. The method of alternative 612, wherein said interleukin-2 is administered at a concentration sufficient to activate the interleukin-2 alpha, beta and gamma receptors.

615. The method of alternative 612, wherein said interleukin-2 is administered at a concentration of 1 IU-1000 IU per million cells.

616. The method of alternative 612, wherein said interleukin-2 is administered at a concentration of 1 IU-100 IU per million cells.

617. The method of alternative 612, wherein said interleukin-2 is administered at a concentration of 1 IU-10 IU per million cells.

618. The method of alternative 611, wherein said cytokine containing media contains interleukin-3.

619. The method of alternative 611, wherein said cytokine containing media contains interleukin-2 followed by a media containing interleukin-3.

620. The method of alternative 611, wherein said cytokine containing media contains interleukin-7.

621. The method of alternative 611, wherein said cytokine containing media contains interleukin-12.

622. The method of alternative 611, wherein said cytokine containing media contains interleukin-15.

623. The method of alternative 611, wherein said cytokine containing media contains interleukin-18.

624. The method of alternative 611, wherein said cytokine containing media is manufactured in a manner to replicate physiological conditions of lymphocyte homeostatic expansion.

625. The method of alternative 624, wherein said cytokine containing media resembling physiological conditions of lymphocyte homeostatic expansion contains interleukin-7 and interleukin-15 at a concentration sufficient to induce proliferation of NK cells independent of antigen recognition.

626. The method of alternative 624, wherein said cytokine containing media resembling physiological conditions of lymphocyte homeostatic expansion contains interleukin-3, interleukin-7 and interleukin-15 at a concentration sufficient to induce proliferation of NK cells independent of antigen recognition.

627. The method of alternative 624, wherein said cytokine containing media resembling physiological conditions of lymphocyte homeostatic expansion contains interleukin-3, interleukin-7, interleukin-15, interleukin-18 at a concentration sufficient to induce proliferation of NK cells independent of antigen recognition.

628. The method of alternative 624, wherein said cytokine containing media resembling physiological conditions of lymphocyte homeostatic expansion contains lithium oxide, interleukin-3, interleukin-7 and interleukin-15 at a concentration sufficient to induce proliferation of NK cells independent of antigen recognition.

629. The method of alternative 624, wherein said cytokine containing media resembling physiological conditions of lymphocyte homeostatic expansion contains a lithium salt, interleukin-3, interleukin-7 and interleukin-15 at a concentration sufficient to induce proliferation of NK cells independent of antigen recognition.

630. The method of alternatives 625 to 629, wherein dendritic cell conditioned media is added to said culture media.

631. The method of alternative 630, wherein said dendritic cell expresses CD83.

632. The method of alternative 630, wherein said dendritic cell expresses CD80.

633. The method of alternative 630, wherein said dendritic cell expresses DEC205.

634. The method of alternative 630, wherein said dendritic cell expresses CD86.

635. The method of alternative 630, wherein said dendritic cell expresses CD40.

636. The method of alternative 630, wherein said dendritic cell expresses interleukin-12 receptor.

637. The method of alternative 630, wherein said dendritic cell expresses interleukin-15 receptor.

638. The method of alternative 630, wherein said dendritic cell is activated by treatment with a double stranded RNA molecule.

639. The method of alternative 638, wherein said double-stranded RNA is Poly IC.

640. The method of alternative 638, wherein said double-stranded RNA is Poly AU.

641. The method of alternative 638, wherein said double-stranded RNA is Poly IC: LC.

642. The method of alternative 630, wherein said dendritic cell is activated by treatment with lipopolysaccharide.

643. The method of alternative 630, wherein said dendritic cell is activated by treatment with neutrophil extracellular traps.

644. The method of alternative 630, wherein said dendritic cell is activated by treatment with unmethylated CpG motifs.

645. The method of alternative 630, wherein said dendritic cell is activated by treatment with beta glucan.

646. The method of alternative 630, wherein said dendritic cell is activated by treatment with TNF-alpha.

647. The method of alternative 630, wherein said dendritic cell is activated by treatment with lymphotoxin.

648. The method of alternative 630, wherein said dendritic cell is activated by treatment with flagellin.

649. The method of alternative 630, wherein said dendritic cell is activated by treatment with HMGB1.

650. The method of alternative 630, wherein said dendritic cell is activated by treatment with pentatraxin.

651. The method of alternative 630, wherein said dendritic cell is activated by treatment with interferon alpha.

652. The method of alternative 630, wherein said dendritic cell is activated by treatment with conditioned media from lectin treated mononuclear blood cells

653. The method of alternative 652, wherein said lectin is phytohemagglutinin.

654. The method of alternative 630, wherein said lectin is concanavalin A.

655. The method of alternative 630, wherein said lectin is pokeweek mitogen.

656. The method of alternative 577, wherein said natural killer cell is derived from a pluripotent stem cell.

657. The method of alternative 656, wherein said pluripotent stem cell is an embryonic stem cell.

658. The method of alternative 657, wherein said embryonic stem cell expresses NANOG.

659. The method of alternative 657, wherein said embryonic stem cell expresses SSEA4.

660. The method of alternative 657, wherein said embryonic stem cell expresses OCT4.

661. The method of alternative 657, wherein said embryonic stem cell expresses SOX-2.

662. The method of alternative 657, wherein said embryonic stem cell expresses TRA-1.

663. The method of alternative 657, wherein said embryonic stem cell expresses interleukin-3 receptor.

664. The method of alternative 657, wherein said embryonic stem cell expresses interleukin-6 receptor.

665. The method of alternative 657, wherein said embryonic stem cell expresses leukemia inhibitory factor receptor.

666. The method of alternative 657, wherein said embryonic stem cell expresses TrK.

667. The method of alternative 657, wherein said embryonic stem cell expresses c-kit.

668. The method of alternative 657, wherein said embryonic stem cell expresses BDNF.

669. The method of alternative 656, wherein said pluripotent stem cell is an inducible pluripotent stem cell.

670. The method of alternative 656, wherein said pluripotent stem cell is a stress derived pluripotent stem cell.

671. The method of alternative 656, wherein said pluripotent stem cell is a somatic cell nuclear transfer derived stem cell.

672. The method of alternative 656, wherein said pluripotent stem cell is a parthenogenic derived stem cell.

673. The method of alternative 577, wherein said NK cell is an immortalized lymphoid cell.

674. The method of alternative 673, wherein said immortalized lymphoid cell is an NK-92 cell.

675. The method of alternative 577, wherein said NK cell is expanded ex vivo in the presence of interleukin-2 provided by a feeder layer of cells.

676. The method of alternative 675, wherein said feeder layer is transfected to stably secrete interleukin-2.

677. The method of alternative 676, wherein said interleukin-2 is produced at a rate of 0.01 IU to 10 IU of interleukin-2 per 10 million transfected fibroblast per 48 hours.

678. The method of alternative 676, wherein said interleukin-2 is produced at a rate of 0.1 IU to 10 IU of interleukin-2 per 10 million transfected fibroblast per 48 hours.

679. The method of alternative 676, wherein said interleukin-2 is produced at a rate of 0.01 IU to 1 IU of interleukin-2 per 10 million transfected fibroblast per 48 hours.

680. The method of alternative 676, wherein said interleukin-2 is produced at a rate of 0.1 IU to 1 IU of interleukin-2 per 10 million transfected fibroblast per 48 hours.

681. The method of alternative 676, wherein said feeder cell layer is mitotically inactivated.

682. The method of alternative 681, wherein said mitotic inactivation is achieved by irradiation.

683. The method of alternative 681, wherein said mitotic inactivation is achieved by chemical means.

684. The method of alternative 683, wherein said chemical inactivation is performed by treatment with mitomycin C.

685. The method of alternative 676, wherein said feeder cell layer is comprised of fibroblasts.

686. The method of alternative 685, wherein said fibroblasts are obtained from keloid tissue.

687. The method of alternative 685, wherein said fibroblasts are obtained from placental tissue.

688. The method of alternative 685, wherein said fibroblasts are obtained from foreskin tissue.

689. The method of alternative 685, wherein said fibroblasts are obtained from amniotic membrane tissue.

690. The method of alternative 685, wherein said fibroblasts are obtained from subintestinal mucosa tissue.

691. The method of alternative 685, wherein said fibroblasts are obtained from endometrial tissue.

692. The method of alternative 685, wherein said fibroblasts are obtained from deciduous tooth tissue.

693. The method of alternative 685, wherein said fibroblasts are obtained from hair follicle tissue.

694. The method of alternative 685, wherein said feeder cells are mesenchymal stem cells.

695. The method of alternative 685, wherein said mesenchymal stem cells express CD90 and/or CD105.

696. The method of alternative 695, wherein said mesenchymal stem cells express CD73.

697. The method of alternative 685, wherein said mesenchymal stem cells express c-kit.

698. The method of alternative 685, wherein said mesenchymal stem cells express c-met.

699. The method of alternative 685, wherein said mesenchymal stem cells express CD55.

700. The method of alternative 685, wherein said mesenchymal stem cells interleukin-18 receptor.

701. The method of alternative 685, wherein said mesenchymal stem cells are derived from the bone marrow.

702. The method of alternative 685, wherein said mesenchymal stem cells are derived from menstrual blood.

703. The method of alternative 685, wherein said mesenchymal stem cells are derived from the chorionic portion of the placenta.

704. The method of alternative 685, wherein said mesenchymal stem cells are derived from peripheral blood.

705. The method of alternative 704, wherein said mesenchymal stem cells are derived from mobilized peripheral blood.

706. The method of alternative 705, wherein said peripheral blood is mobilized by administration of flt-3 ligand.

707. The method of alternative 705, wherein said peripheral blood is mobilized by administration of G-CSF.

708. The method of alternative 705, wherein said peripheral blood is mobilized by administration of GM-CSF.

709. The method of alternative 705, wherein said peripheral blood is mobilized by administration of M-CSF.

710. The method of alternative 705, wherein said peripheral blood is mobilized by administration of a CXCR4 antagonist.

711. The method of alternative 675, wherein said feeder layer is transfected to stably secrete interleukin-7.

712. The method of alternative 675, wherein said feeder layer is transfected to stably secrete interleukin-2 and interleukin-7.

713. The method of alternative 675, wherein said feeder layer is transfected to stably secrete interleukin-15.

714. The method of alternative 675, wherein said feeder layer is transfected to stably secrete interleukin-2 and interleukin-15.

715. The method of alternative 675, wherein said feeder layer is transfected to stably secrete interleukin-7 and interleukin-15.

716. The method of alternative 675, wherein said feeder layer is transfected to stably secrete interleukin-2, interleukin-7, and interleukin-15.

717. The method of alternative 716, wherein said feeder layer is further transfected to express an activator of natural killer cell activator receptor.

718. The method of alternative 717, wherein said activator of said natural killer cell activator receptor is MICA.

719. The method of alternative 717, wherein said activator of said natural killer cell activator receptor is calreticulin.

720. The method of alternative 717, wherein said activator of said natural killer cell activator receptor is ULBP-1.

721. The method of alternative 717, wherein said activator of said natural killer cell activator receptor is ULBP-2.

722. The method of alternative 717, wherein said activator of said natural killer cell activator receptor is ULBP-5.

723. The method of alternative 717, wherein said activator of said natural killer cell activator receptor is ULBP-6.

724. The method of alternative 717, wherein said activator of said natural killer cell activator receptor is ULBP-3.

725. The method of alternative 717, wherein said activator of said natural killer cell activator receptor is MICB.

726. The method of alternative 717, wherein an antigen presenting cell is added to the culture in order to present NK activating antigens.

727. The method of alternative 726, wherein said antigen presenting cell is a B cell.

728. The method of alternative 726, wherein said antigen presenting cell is an endothelial cell.

729. The method of alternative 728, wherein said endothelial cell is treated in a manner to enhance antigen presenting activity.

730. The method of alternative 730, wherein said enhancement of antigen presenting activity is accomplished by transfection with interleukin-2.

731. The method of alternative 730, wherein said enhancement of antigen presenting activity is accomplished by transfection with transporter associated protein-1.

732. The method of alternative 730, wherein said enhancement of antigen presenting activity is accomplished by transfection with antisense oligonucleotides to interleukin-10.

733. The method of alternative 730, wherein said enhancement of antigen presenting activity is accomplished by transfection with antisense oligonucleotides to interleukin-4.

734. The method of alternative 730, wherein said enhancement of antigen presenting activity is accomplished by transfection with antisense oligonucleotides to interleukin-35.

735. The method of alternative 730, wherein said enhancement of antigen presenting activity is accomplished by transfection with antisense oligonucleotides to TGF-beta.

736. The method of alternative 730, wherein said enhancement of antigen presenting activity is accomplished by transfection with antisense oligonucleotides to interleukin-13.

737. The method of alternative 730, wherein said enhancement of antigen presenting activity is accomplished by transfection with shRNA to interleukin-10.

738. The method of alternative 730, wherein said enhancement of antigen presenting activity is accomplished by transfection with shRNA to interleukin-4.

739. The method of alternative 730, where said enhancement of antigen presenting activity is accomplished by transfection with shRNA to interleukin-35.

740. The method of alternative 730, wherein said enhancement of antigen presenting activity is accomplished by transfection with shRNA to TGF-beta.

741. The method of alternative 730, wherein said enhancement of antigen presenting activity is accomplished by transfection with shRNA to interleukin-13.

742. The method of alternative 730, wherein said enhancement of antigen presenting activity is accomplished by transfection with interleukin-6.

743. The method of alternative 730, wherein said enhancement of antigen presenting activity is accomplished by transfection with interleukin-7

744. The method of alternative 730, wherein said enhancement of antigen presenting activity is accomplished by transfection with interleukin-8.

745. The method of alternative 730, wherein said enhancement of antigen presenting activity is accomplished by transfection with interleukin-12.

746. The method of alternative 730, wherein said enhancement of antigen presenting activity is accomplished by transfection with interleukin-15.

747. The method of alternative 730, wherein said enhancement of antigen presenting activity is accomplished by transfection with interleukin-18.

748. The method of alternative 730, wherein said enhancement of antigen presenting activity is accomplished by transfection with interleukin-21.

747. The method of alternative 730, wherein said enhancement of antigen presenting activity is accomplished by transfection with interleukin-23.

748. The method of alternative 730, wherein said enhancement of antigen presenting activity is accomplished by transfection with interleukin-27.

749. The method of alternative 730, wherein said enhancement of antigen presenting activity is accomplished by transfection with interleukin-33.

750. The method of alternative 730, wherein said enhancement of antigen presenting activity is accomplished by transfection with HMGB1.

751. The method of alternative 730, wherein said enhancement of antigen presenting activity is accomplished by transfection with TNF-alpha.

752. The method of alternative 730, wherein said enhancement of antigen presenting activity is accomplished by transfection with RANK ligand.

753. The method of alternative 730, wherein said enhancement of antigen presenting activity is accomplished by transfection with osteopontin.

754. The method of alternative 730, wherein said enhancement of antigen presenting activity is accomplished by transfection with BlyS.

755. The method of alternative 730, wherein said enhancement of antigen presenting activity is accomplished by transfection with TRAIL.

756. The method of alternative 730, wherein said enhancement of antigen presenting activity is accomplished by transfection with TRANCE.

757. The method of alternative 730, wherein said enhancement of antigen presenting activity is accomplished by transfection with interferon alpha.

758. The method of alternative 730, wherein said enhancement of antigen presenting activity is accomplished by transfection with interferon beta.

759. The method of alternative 730, wherein said enhancement of antigen presenting activity is accomplished by transfection with interferon gamma.

760. The method of alternative 730, wherein said enhancement of antigen presenting activity is accomplished by transfection with interferon tau.

761. The method of alternative 730, wherein said enhancement of antigen presenting activity is accomplished by transfection with TLR4.

762. The method of alternative 730, wherein said enhancement of antigen presenting activity is accomplished by transfection with TLR2.

763. The method of alternative 730, wherein said enhancement of antigen presenting activity is accomplished by transfection with TLR7/8.

764. The method of alternative 730, wherein said enhancement of antigen presenting activity is accomplished by transfection with TLR9.

765. The method of alternative 730, wherein said enhancement of antigen presenting activity is accomplished by transfection with RIG-1.

766. The method of alternative 730, wherein said enhancement of antigen presenting activity is accomplished by transfection with MDA-5.

767. The method of alternative 730, wherein said enhancement of antigen presenting activity is accomplished by transfection with calreticulin.

768. The method of alternative 730, wherein said enhancement of antigen presenting activity is accomplished by transfection with hsp65.

769. The method of alternative 730, wherein said enhancement of antigen presenting activity is accomplished by transfection with gp-96.

770. The method of alternative 730, wherein said enhancement of antigen presenting activity is accomplished by transfection with CD5.

771. The method of alternative 730, wherein said enhancement of antigen presenting activity is accomplished by transfection with CD40.

772. The method of alternative 730, wherein said enhancement of antigen presenting activity is accomplished by transfection with CD80.

773. The method of alternative 730, wherein said enhancement of antigen presenting activity is accomplished

774. The method of alternative 730, wherein said enhancement of antigen presenting activity is accomplished by transfection with DEC-205.

775. The method of alternative 730, wherein said enhancement of antigen presenting activity is accomplished by transfection with CD83

776. The method of alternative 577, wherein said natural killer cells are extracted from peripheral blood CD56 cells and expanded ex vivo in a media containing one or more agents capable of inducing dedifferentiation.

777. The method of alternative 776, where said agent capable of inducing dedifferentiation is cytoplasm from a cell possessing an earlier differentiation status than said NK cell.

778. The method of alternative 777, wherein said cell possessing an earlier differentiation status than said NK cell is a cell expressing CD133.

779. The method of alternative 777, wherein said cell possessing an earlier differentiation status than said NK cell is a cell expressing CD34.

780. The method of alternative 777, wherein said cell possessing an earlier differentiation status than said NK cell is a cell expressing steel factor receptor.

781. The method of alternative 777, wherein said cell possessing an earlier differentiation status than said NK cell is a cell expressing c-met.

782. The method of alternative 778, wherein said cell expressing CD133 is a hematopoietic stem cell.

783. The method of alternative 778, wherein said cell expressing CD133 is a hepatogenic stem cell.

784. The method of alternative 778, wherein said cell expressing CD133 is a neurogenic stem cell.

785. The method of alternative 778, wherein said cell expressing CD133 is an endothelial progenitor cell.

786. The method of alternative 778, wherein said cell expressing CD133 is a trophoblastic stem cell.

787. The method of alternative 778, wherein said cell expressing CD133 is a mesenchymal stem cell.

788. The method of alternative 778, wherein said cell expressing CD133 is an endometrial stem cell.

789. The method of alternative 778, wherein said cell expressing CD133 is a NK progenitor cell.

790. The method of alternative 778, wherein said cell expressing CD133 is a deciduous tooth stem cell.

791. The method of alternative 778, wherein said cell expressing CD133 is a cord blood stem cell.

792. The method of alternative 778, wherein said cell expressing CD133 is a dermal stem cell.

793. The method of alternative 778, wherein said cell expressing CD133 is a fallopian tube stem cell.

794. The method of alternative 778, wherein said cell expressing CD133 is a hair follicle stem cell.

795. The method of alternative 778, wherein said cell expressing CD133 is a cardiogenic stem cell.

796. The method of alternative 778, wherein said cell expressing CD133 is a type II pulmonary epithelial cell.

797. The method of alternative 778-796, wherein cytoplasm from said cells is introduced to the cytoplasm of NK cells to be expanded.

798. The method of alternative 797, wherein said cytoplasm of said dedifferentiated cell is introduced into said NK cell to be expanded by means of electroporation.

799. The method of alternative 797, wherein said cytoplasm of said dedifferentiated cell is introduced into said NK cell to be expanded by means of ultrasound membrane disruption.

800. The method of alternative 797, wherein said cytoplasm of said dedifferentiated cell is introduced into said NK cell to be expanded by means of streptolysin O membrane permeabilization.

801. The method of alternative 797, wherein said cytoplasm of said dedifferentiated cell is introduced into said NK cell to be expanded by means of limited membrane attack complex activation.

802. The method of alternative 797, wherein said cytoplasm of said dedifferentiated cell is introduced into said NK cell to be expanded by means of osmotic pulse.

803. The method of alternative 577, wherein said NK cells are fused with a cell of a more primitive differentiation status.

804. The method of alternative 803, wherein said cell of a more primitive differentiation status is an oocyte.

805. The method of alternative 803, wherein said cell of a more primitive differentiation status is an oocyte precursor cell.

806. The method of alternative 803, wherein said cell of a more primitive differentiation status is a Leydig cell.

807. The method of alternative 803, wherein said cell of a more primitive differentiation status is a Sertoli cell.

808. The method of alternative 803, wherein said cell of a more primitive differentiation status is a Sertoli cell purified based on expression of Fas ligand.

809. The method of alternative 803, wherein said cell of a more primitive differentiation status is a Sertoli cell purified based on expression of CD25.

810. The method of alternative 803, wherein said cell of a more primitive differentiation status is a Sertoli cell purified based on expression of GITR ligand.

811. The method of alternative 803, wherein said cell of a more primitive differentiation status is a Sertoli cell purified based on expression of NGF receptor.

812. The method of alternative 803, wherein said cell of a more primitive differentiation status is a Sertoli cell purified based on expression of CD123.

813. The method of alternative 803, wherein said cell of a more primitive differentiation status is a Sertoli cell purified based on expression of GDNF receptor.

814. The method of alternative 803, wherein said cell of a more primitive differentiation status is a Sertoli cell purified based on expression of c-kit.

815. The method of alternative 803, wherein said cell of a more primitive differentiation status is a hematopoietic stem cell.

816. The method of alternative 815, wherein said hematopoietic stem cell is purified from the bone marrow.

817. The method of alternative 815, wherein said hematopoietic stem cell is purified from peripheral blood.

818. The method of alternative 815, wherein said hematopoietic stem cell is purified from mobilized peripheral blood.

819. The method of alternative 815, wherein said hematopoietic stem cell is purified from cord blood.

820. The method of alternative 815, wherein said hematopoietic stem cell is purified from menstrual blood.

821. The method of alternative 815, wherein said hematopoietic stem cell expresses CD34.

822. The method of alternative 815, wherein said hematopoietic stem cell expresses CD133.

823. The method of alternative 815, wherein said hematopoietic stem cell expresses interleukin-3 receptor.

824. The method of alternative 815, wherein said hematopoietic stem cell is capable of reconstituting the myeloid lineage in an immunodeficient animal.

825. The method of alternative 815, wherein said hematopoietic stem cell is capable of reconstituting the lymphoid lineage in an immunodeficient animal.

826. The method of alternative 815, wherein said hematopoietic stem cell is capable of reconstituting the erythroid lineage in an immunodeficient animal.

827. The method of alternative 815, wherein said hematopoietic stem cell is capable of reconstituting the thrombocytic lineage in an immunodeficient animal.

828. The method of alternative 815, wherein said hematopoietic stem cell is capable of reconstituting all hematopoietic lineages in an immunodeficient animal.

829. The method of alternative 803, wherein said cell of a more primitive differentiation status is a pluripotent stem cell.

830. The method of alternative 803, wherein said cell of a more primitive differentiation status is an induced pluripotent stem cell.

831. The method of alternative 803, wherein said cell of a more primitive differentiation status is a mesenchymal stem cell.

832. The method of alternative 831, wherein said mesenchymal stem cell is a plastic adherent cell.

833. The method of alternative 831, wherein said mesenchymal stem cell expresses STRO-1.

834. The method of alternative 831, wherein said mesenchymal stem cell is extracted from a tissue based on expression of CD55.

835. The method of alternative 834, wherein said tissue is umbilical cord tissue.

836. The method of alternative 834, wherein said tissue is bone marrow. 837. The method of alternative 834, wherein said tissue is endometrium.

838. The method of alternative 834, wherein said tissue is placenta.

839. The method of alternative 834, wherein said tissue is amniotic fluid.

840. The method of alternative 834, wherein said tissue is amniotic membrane.

841. The method of alternative 834, wherein said tissue is ovarian tissue. 842. The method of alternative 834, wherein said tissue is testicular tissue.

843. The method of alternative 834, wherein said tissue is adipose tissue.

844. The method of alternative 834, wherein said tissue is omental tissue.

845. The method of alternative 835, wherein said umbilical cord tissue is perfused with media containing 1% v/v carbon monoxide in a saline solution prior to extraction of said cells.

846. The method of alternative 835, wherein said umbilical cord tissue is perfused with media containing 2% v/v carbon monoxide in a saline solution prior to extraction of said cells.

847. The method of alternative 835, wherein said umbilical cord tissue is perfused with media containing 1-5% v/v carbon monoxide in a saline solution prior to extraction of said cells.

848. The method of alternative 835, wherein said umbilical cord tissue is perfused with media containing 1% v/v argon in a saline solution prior to extraction of said cells.

849. The method of alternative 835, wherein said umbilical cord tissue is perfused with media containing 2% v/v argon in a saline solution prior to extraction of said cells.

850. The method of alternative 835, wherein said umbilical cord tissue is perfused with media containing 1-5% v/v argon in a saline solution prior to extraction of said cells.

851. The method of alternative 835, wherein said umbilical cord tissue is perfused with media containing 1% v/v xenon in a saline solution prior to extraction of said cells.

852. The method of alternative 835, wherein said umbilical cord tissue is perfused with media containing 2% v/v xenon in a saline solution prior to extraction of said cells.

853. The method of alternative 835, wherein said umbilical cord tissue is perfused with media containing 1-5% v/v xenon in a saline solution prior to extraction of said cells.

854. The method of alternative 835, wherein said umbilical cord tissue is perfused with media containing 1% v/v krypton in a saline solution prior to extraction of said cells.

855. The method of alternative 835, wherein said umbilical cord tissue is perfused with media containing 2% v/v krypton in a saline solution prior to extraction of said cells.

856. The method of alternative 835, wherein said umbilical cord tissue is perfused with media containing 1-5% v/v krypton in a saline solution prior to extraction of said cells.

857. The method of alternative 835, wherein said umbilical cord tissue is perfused with media containing 1% v/v xenon in a saline solution prior to extraction of said cells.

858. The method of alternative 835, wherein said umbilical cord tissue is perfused with media containing 2% v/v xenon in a saline solution prior to extraction of said cells.

859. The method of alternative 835, wherein said umbilical cord tissue is perfused with media containing 1-5% v/v xenon in a saline solution prior to extraction of said cells.

860. The method of alternative 835, wherein said umbilical cord tissue is perfused with media containing 1% v/v helium in a saline solution prior to extraction of said cells.

861. The method of alternative 835, wherein said umbilical cord tissue is perfused with media containing 2% v/v helium in a saline solution prior to extraction of said cells.

862. The method of alternative 835, wherein said umbilical cord tissue is perfused with media containing 1-5% v/v hydrogen in a saline solution prior to extraction of said cells.

863. The method of alternative 835, wherein said umbilical cord tissue is perfused with media containing 1% v/v hydrogen in a saline solution prior to extraction of said cells.

864. The method of alternative 835, wherein said umbilical cord tissue is perfused with media containing 2% v/v hydrogen in a saline solution prior to extraction of said cells.

865. The method of alternative 835, wherein said umbilical cord tissue is perfused with media containing 1-5% v/v helium in a saline solution prior to extraction of said cells.

866. The method of alternative 831, wherein said mesenchymal stem cells are pre-treated with an regenerative adjuvant prior to fusion.

867. The method of alternative 866, wherein said regenerative adjuvant is platelet rich plasma.

868. The method of alternative 866, wherein said regenerative adjuvant is platelet rich fibrin.

869. The method of alternative 866, wherein said regenerative adjuvant is conditioned media from activated T cells.

870. The method of alternative 869, wherein said T cells are activated by stimulation of T cell receptor signaling.

871. The method of alternative 870, wherein said stimulation of T cell receptor is accomplished through induction of calcium flux.

872. The method of alternative 870, wherein said stimulation of T cell receptor is accomplished through crosslinking of T cell receptor.

872. The method of alternative 870, wherein said stimulation of T cell receptor is accomplished through crosslinking of CD3.

873. The method of alternative 870, wherein said stimulation of T cell receptor is accomplished through crosslinking of CD3 and CD28.

874. The method of alternative 870, wherein said stimulation of T cell receptor is accomplished through crosslinking of CD3 in the presence of interleukin-2.

875. The method of alternative 870, wherein said stimulation of T cell receptor is accomplished through crosslinking of CD3 and CD28 in the presence of interleukin-2.

876. The method of alternative 870, wherein said stimulation of T cell receptor is accomplished through contacting with an allogeneic antigen presenting cell.

877. The method of alternative 876, wherein said allogeneic antigen presenting cell is an endothelial cell.

878. The method of alternative 876, wherein said allogeneic antigen presenting cell is a neutrophil.

879. The method of alternative 876, wherein said allogeneic antigen presenting cell is a monocyte.

880. The method of alternative 876, wherein said allogeneic antigen presenting cell is a T cell.

881. The method of alternative 876, wherein said allogeneic antigen presenting cell is a dendritic cell.

882. The method of alternative 876, wherein said allogeneic antigen presenting cell is a myeloid dendritic cell.

883. The method of alternative 876, wherein said allogeneic antigen presenting cell is a lymphoid dendritic cell.

884. The method of alternative 876, wherein said allogeneic antigen presenting cell is a NKT cell.

885. The method of alternative 876, wherein said allogeneic antigen presenting cell is a gamma delta T cell.

886. The method of alternative 876, wherein said allogeneic antigen presenting cell is a basophil.

887. The method of alternative 876, wherein said allogeneic antigen presenting cell is an eosinophil.

888. The method of alternative 876, wherein said allogeneic antigen presenting cell is a mast cell.

889. The method of alternative 870, wherein said stimulation of T cell receptor is accomplished through exposure of said T cell to a mitogen.

890. The method of alternative 889, wherein said mitogen is a lectin.

891. The method of alternative 890, wherein said lectin is a sugar binding protein.

892. The method of alternative 890, wherein said lectin is concanavalin A.

893. The method of alternative 890, wherein said lectin is a PHA.

894. The method of alternative 890, wherein said lectin is a CNA.

895. The method of alternative 890, wherein said lectin is a sugar binding protein.

896. The method of alternative 866, wherein said regenerative adjuvant is interleukin-1.

897. The method of alternative 866, wherein said regenerative adjuvant is interleukin-3.

898. The method of alternative 866, wherein said regenerative adjuvant is interleukin-4.

899. The method of alternative 866, wherein said regenerative adjuvant is interleukin-10.

900. The method of alternative 866, wherein said regenerative adjuvant is interleukin-13.

901. The method of alternative 866, wherein said regenerative adjuvant is interleukin-16.

902. The method of alternative 866, wherein said regenerative adjuvant is interleukin-20.

903. The method of alternative 866, wherein said regenerative adjuvant is angiopoietin.

904. The method of alternative 866, wherein said regenerative adjuvant is interleukin-22.

906. The method of alternative 866, wherein said regenerative adjuvant is interleukin-35.

907. The method of alternative 866, wherein said regenerative adjuvant is interleukin-38.

908. The method of alternative 866, wherein said regenerative adjuvant is TGF-beta.

910. The method of alternative 866, wherein said regenerative adjuvant is activin.

911. The method of alternative 866, wherein said regenerative adjuvant is endoglin.

912. The method of alternative 866, wherein said regenerative adjuvant is hematopoietic stem cell conditioned media.

913. The method of alternative 912, wherein said hematopoietic stem cell conditioned media is generated by exposing hematopoietic stem cells to an inflammatory stimuli and collecting conditioned media.

914. The method of alternative 913, wherein said inflammatory stimuli is a toll like receptor activator.

915. The method of alternative 914, wherein said toll like receptor activator is OK-342.

916. The method of alternative 914, wherein said toll like receptor activator is beta glucan.

917. The method of alternative 914, wherein said toll like receptor activator is HMGB1.

918. The method of alternative 914, wherein said toll like receptor activator is neutrophil extracellular traps.

919. The method of alternative 914, wherein said toll like receptor activator is bacterial membrane extract.

920. The method of alternative 914, wherein said toll like receptor activator is lipopolysaccharide.

921. The method of alternative 914, wherein said toll like receptor activator is Poly IC.

922. The method of alternative 914, wherein said toll like receptor activator is Poly AU.

923. The method of alternative 914, wherein said toll like receptor activator is Poly IC: LC.

924. The method of alternative 914, wherein said toll like receptor activator is flagellin

925. The method of alternative 914, wherein said toll like receptor activator is gp96.

926. The method of alternative 914, wherein said toll like receptor activator is histone H4.

927. The method of alternative 914, wherein said toll like receptor activator is calreticulin.

928. The method of alternative 914, wherein said toll like receptor activator is uric acid crystals.

929. The method of alternative 913, wherein said hematopoietic stem cell possesses interleukin-3 receptor.

930. The method of alternative 913, wherein said hematopoietic stem cells possess HGF-1 receptor.

931. The method of alternative 913, wherein said hematopoietic stem cells possess interleukin-6 receptor.

932. The method of alternative 913, wherein said hematopoietic stem cells possess interleukin-8 receptor.

933. The method of alternative 913, wherein said hematopoietic stem cells possess interleukin-10 receptor.

934. The method of alternative 913, wherein said hematopoietic stem cells possess interleukin-11 receptor.

935. The method of alternative 913, wherein said hematopoietic stem cells possess interleukin-20 receptor.

936. The method of alternative 913, wherein said hematopoietic stem cells possess thrombopoietin receptor.

937. The method of alternative 913, wherein said hematopoietic stem cells possess FGF-1 receptor.

938. The method of alternative 913, wherein said hematopoietic stem cells possess FGF-2 receptor.

939. The method of alternative 913, wherein said hematopoietic stem cells possess FGF-5 receptor.

940. The method of alternative 913, wherein said hematopoietic stem cells possess TGF-beta receptor.

941. The method of alternative 913, wherein said hematopoietic stem cells possess endoglin receptor.

942. The method of alternative 913, wherein said hematopoietic stem cells possess the ability to produce autocrine TGF-beta.

943. The method of alternative 913, wherein said hematopoietic stem cells possess leukemia inhibitory factor receptor.

944. The method of alternative 913, wherein said hematopoietic stem cells possess VEGF receptor.

945. The method of alternative 913, wherein said hematopoietic stem cells possess PGE2 receptor.

946. The method of alternative 913, wherein said hematopoietic stem cells possess interferon gamma receptor.

947. The method of alternative 913, wherein said hematopoietic stem cells express RANK ligand.

948. The method of alternative 913, wherein said hematopoietic stem cells possess CD34.

949. The method of alternative 913, wherein said hematopoietic stem cells possess CD133.

950. The method of alternative 913, wherein said hematopoietic stem cells possess CD34 and CD133.

951. The method of alternative 913, wherein said hematopoietic stem cells lack expression of CD38.

952. The method of alternative 913, wherein said hematopoietic stem cells possess interleukin-3 receptor.

953. The method of alternative 577, wherein said natural killer cells are extracted from tissue.

954. The method of alternative 953, wherein said tissue derived natural killer cells are isolated from a group of tissues comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; 1) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

955. The method of alternative 577, wherein said tissue derived natural killer cells are isolated from tissues containing cells selected from a group of cells comprising of: endothelial cells, epithelial cells, d8rmal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, Macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

956. The method of alternative 577, wherein said NK cells are expanded ex vivo by culture in the presence of a histone deacetylase inhibitor.

957. The method of alternative 956, wherein said histone deacetylase inhibitor is vorinostat.

958. The method of alternative 956, wherein said histone deacetylase inhibitor is entinostat.

959. The method of alternative 956, wherein said histone deacetylase inhibitor is trichostatin A.

960. The method of alternative 956, wherein said histone deacetylase inhibitor is mocetinostat.

961. The method of alternative 956, wherein said histone deacetylase inhibitor is belinostat.

962. The method of alternative 956, wherein said histone deacetylase inhibitor is romidepsin.

963. The method of alternative 956, wherein said histone deacetylase inhibitor is MC1568.

964. The method of alternative 956, wherein said histone deacetylase inhibitor is tubastatin.

965. The method of alternative 956, wherein said histone deacetylase inhibitor is givinostat.

966. The method of alternative 956, wherein said histone deacetylase inhibitor is dacinostat.

967. The method of alternative 956, wherein said histone deacetylase inhibitor is CUDC1.

968. The method of alternative 956, wherein said histone deacetylase inhibitor is quisinostat.

969. The method of alternative 956, wherein said histone deacetylase inhibitor is pracinostat.

970. The method of alternative 956, wherein said histone deacetylase inhibitor is PCI-34151.

971. The method of alternative 956, wherein said histone deacetylase inhibitor is SKLB-23bb.

972. The method of alternative 956, wherein said histone deacetylase inhibitor is TH34.

973. The method of alternative 956, wherein said histone deacetylase inhibitor is suberohydroxamic acid.

974. The method of alternative 956, wherein said histone deacetylase inhibitor is UF010.

975. The method of alternative 956, wherein said histone deacetylase inhibitor is WT161.

976. The method of alternative 1, wherein subsequent to ablative therapy immunization with tumor-specific antigens is performed.

977. The method of alternative 976, wherein said tumor specific antigens are derived from heat shock proteins bound to tumor cell lysates.

978. The method of alternative 976, wherein said tumor specific antigens are derived from tumor cell lysates.

979. The method of alternative 976, wherein said tumor specific antigens are derived from tumor cell mRNA.

980. The method of alternative 976, wherein said tumor specific antigens are derived from tumor cell amplified DNA.

981. The method of alternative 976, wherein said tumor specific antigens are derived from tumor cell exosomes.

982. The method of alternative 976, wherein said tumor specific antigens are derived from tumor derived stem cells.

983. The method of alternative 982, wherein said tumor derived stem cells are isolated from tumor mass by isolation of cells possessing markers associated with regenerative cells.

984. The method of alternative 983, wherein said marker associated with tumor stem cells is CD34.

985. The method of alternative 983, wherein said marker associated with tumor stem cells is CD133.

986. The method of alternative 982, wherein said tumor specific antigen is ERG.

987. The method of alternative 982, wherein said tumor specific antigen is WT1.

988. The method of alternative 982, wherein said tumor specific antigen is BCR-ABL.

989. The method of alternative 982, wherein said tumor specific antigen is MUC1.

990. The method of alternative 982, wherein said tumor specific antigen is MYC-N.

991. The method of alternative 982, wherein said tumor specific antigen is hTERT.

992. The method of alternative 982, wherein said tumor specific antigen is LMP2.

993. The method of alternative 982, wherein said tumor specific antigen is survivin.

994. The method of alternative 982, wherein said tumor specific antigen is cyclin B1.

995. The method of alternative 982, wherein said tumor specific antigen is EGFR vII.

996. The method of alternative 982, wherein said tumor specific antigen is EGFR vIII.

997. The method of alternative 982, wherein said tumor specific antigen is RhoC.

998. The method of alternative 982, wherein said tumor specific antigen is B cell idiotype.

999. The method of alternative 982, wherein said tumor specific antigen is T cell idiotype.

1000. The method of alternative 982, wherein said tumor specific antigen is IAP.

1001. The method of alternative 982, wherein said tumor specific antigen is selected from a group comprising of: PLAC1, HPV E6, HPV E7, OY-TES1, Her2/neu, PAX3, NY-BR-1, p53 mutant, MAGE A3, EpCAM, polysialic Acid, AFP, PAX5, NY-ESO1, sperm protein 17, GD3, Fucosyl GM1, mesothelin, PSMA, GD2, MAGE A1, sLe(x), HMWMAA, CYP1B1, sperm fibrous sheath protein, B7H3, TRP-2, AKAP-4, XAGE 1, CEA, Tn, GloboH, SSX2, RGS5, SART3, gp100, MelanA/MART1, Tyrosinase, GM3 ganglioside, Proteinase 3 (PR1), Page4, STn, Carbonic anhydrase IX, PSCA, Legumain, MAD-CT-1 (protamin2), PSA, Tie 2, MAD-CT2, PAP, PDGFR-beta, NA17, VEGFR2, FAP, LCK, Fos-related antigen, LCK, FAP.

1002. A method of predicting a cancer patient's response to therapy comprising the steps of: a) obtaining a tumor sample from said cancer patient; b) exposing said tumor samples to a matrix replicating conditions found inside said tumor microenvironment; c) exposing said tumor sample in said conditions replicating said cancer microenvironment to various cancer treatments being contemplated for us in said cancer patients; d) observing effects of said treatments on said cancer cells; and e) recommending to caregiver of said patient which treatments have evoked an in vitro response.

1003. The method of alternative 1002, wherein said tumor sample is obtained by biopsy.

1004. The method of alternative 1002, wherein said tumor sample is obtained from peripheral blood.

1005. The method of alternative 1004, wherein said tumor sample is obtained as circulating tumor cells from peripheral blood.

1006. The method of alternative 1005, wherein said circulating tumor cells are selected based on expression of PECAM.

1007. The method of alternative 1005, wherein said circulating tumor cells are selected based on expression of CD133.

1008. The method of alternative 1005, wherein said circulating tumor cells are selected based on expression of CD34.

1009. The method of alternative 1005, wherein said circulating tumor cells are selected based on expression of stem cell factor receptor.

1010. The method of alternative 1005, wherein said circulating tumor cells are selected based on expression of c-met.

1011. The method of alternative 1005, wherein said circulating tumor cells are selected based on expression of TGF-beta receptor.

1012. The method of alternative 1005, wherein said circulating tumor cells are selected based on expression of interleukin-1 receptor.

1013. The method of alternative 1005, wherein said circulating tumor cells are selected based on expression of interleukin-17 receptor.

1014. The method of alternative 1005, wherein said circulating tumor cells are selected based on expression of mucin-1.

1015. The method of alternative 1005, wherein said circulating tumor cells are selected based on expression of PECAM.

1016. The method of alternative 1005, wherein said circulating tumor cells are selected based on ability to export rhodamine 231.

1017. The method of alternative 1005, wherein said circulating tumor cells are selected based expression of LIF receptor.

1018. The method of alternative 1005, wherein said circulating tumor cells are selected based expression of IL-10 receptor.

1019. The method of alternative 1005, wherein said circulating tumor cells are selected based enhanced binding to lectins.

1020. The method of alternative 1005, wherein said circulating tumor cells are selected based expression of LIF receptor.

1021. The method alternative 1002, wherein said tumor sample is a tumor derived microvesicle.

1022. The method of alternative 1021, wherein said microvesicle is between 20 nm-300 nm in size.

1023. The method of alternative 1021, wherein said microvesicle is between 50 nm-250 nm in size.

1024. The method of alternative 1021, wherein said microvesicle is between 70 nm-150 nm in size.

1025. The method of alternative 1021, wherein said microvesicle is an exosome.

1026. The method of alternative 1025, wherein said exosome expresses annexin-V.

1027. The method of alternative 1025, wherein said exosome expresses CD9.

1028. The method of alternative 1025, wherein said exosome expresses PDCD6IP.

1029. The method of alternative 1025, wherein said exosome expresses HSPA8.

1030. The method of alternative 1025, wherein said exosome expresses GAPDH.

1031. The method of alternative 1025, wherein said exosome expresses ACTB.

1032. The method of alternative 1025, wherein said exosome expresses ANXA2.

1033. The method of alternative 1025, wherein said exosome expresses CD63.

1034. The method of alternative 1025, wherein said exosome expresses SDCBP.

1035. The method of alternative 1025, wherein said exosome expresses ENO1.

1036. The method of alternative 1025, wherein said exosome expresses HSP90AA1.

1037. The method of alternative 1025, wherein said exosome expresses TSG101.

1038. The method of alternative 1025, wherein said exosome expresses PKM.

1039. The method of alternative 1025, wherein said exosome expresses LDHA.

1040. The method of alternative 1025, wherein said exosome expresses EEF1A1.

1041. The method of alternative 1025, wherein said exosome expresses YWHAZ.

1042. The method of alternative 1025, wherein said exosome expresses PGK1.

1043. The method of alternative 1025, wherein said exosome expresses EEF2.

1044. The method of alternative 1025, wherein said exosome expresses ALDOA.

1045. The method of alternative 1025, wherein said exosome expresses HSP90AB1.

1046. The method of alternative 1025, wherein said exosome expresses ANXA5.

1047. The method of alternative 1025, wherein said exosome expresses FASN.

1048. The method of alternative 1025, wherein said exosome expresses YWHAE.

1049. The method of alternative 1025, wherein said exosome expresses CLTC.

1050. The method of alternative 1025, wherein said exosome expresses CD81.

1051. The method of alternative 1025, wherein said exosome expresses GNAS.

1052. The method of alternative 1025, wherein said exosome expresses RAB5C.

1053. The method of alternative 1025, wherein said exosome expresses ARF1.

1054. The method of alternative 1025, wherein said exosome expresses ANXA6.

1055. The method of alternative 1025, wherein said exosome expresses ANXA11.

1056. The method of alternative 1025, wherein said exosome expresses ACTG1.

1057. The method of alternative 1025, wherein said exosome expresses KPNB1.

1058. The method of alternative 1025, wherein said exosome expresses EZR.

1059. The method of alternative 1025, wherein said exosome expresses ANXA4.

1060. The method of alternative 1025, wherein said exosome expresses LIF receptor.

1061. The method of alternative 1025, wherein said exosome expresses interleukin-1 receptor antagonist membrane bound.

1062. The method of alternative 1025, wherein said exosome expresses placental growth factor receptor.

1063. The method of alternative 1025, wherein said exosome expresses LDHB.

1064. The method of alternative 1025, wherein said exosome expresses RAB7A.

1065. The method of alternative 1025, wherein said exosome expresses YWHAB.

1066. The method of alternative 1025, wherein said exosome expresses ACTN4.

1067. The method of alternative 1025, wherein said exosome expresses EHD4.

1068. The method of alternative 1025, wherein said exosome expresses GDI2.

1069. The method of alternative 1025, wherein said exosome expresses PRDX2.

1070. The method of alternative 1025, wherein said exosome expresses MFGE8.

1071. The method of alternative 1025, wherein said exosome expresses RHOA.

1072. The method of alternative 1025, wherein said exosome expresses ANXA1.

1073. The method of alternative 1025, wherein said exosome expresses GNAI2.

1074. The method of alternative 1025, wherein said exosome expresses HSPA1A.

1075. The method of alternative 1025, wherein said exosome expresses LGALS3BP.

1076. The method of alternative 1025, wherein said exosome expresses RAC1.

1077. The method of alternative 1025, wherein said exosome expresses TUBA1B.

1078. The method of alternative 1025, wherein said exosome expresses A2M.

1079. The method of alternative 1025, wherein said exosome expresses YWHAG.

1080. The method of alternative 1025, wherein said exosome expresses CDC42.

1081. The method of alternative 1025, wherein said exosome expresses CCT2.

1082. The method of alternative 1025, wherein said exosome expresses CLIC1.

1083. The method of alternative 1025, wherein said exosome expresses FLNA.

1084. The method of alternative 1025, wherein said exosome expresses FLOT1.

1085. The method of alternative 1025, wherein said exosome expresses YWHAQ.

1086. The method of alternative 1025, wherein said exosome expresses ATP1A1.

1087. The method of alternative 1025, wherein said exosome expresses GNB2.

1088. The method of alternative 1025, wherein said exosome expresses HIST1H4A.

1089. The method of alternative 1025, wherein said exosome expresses SLC3A2.

1090. The method of alternative 1025, wherein said exosome expresses HSPA5.

1091. The method of alternative 1025, wherein said exosome expresses ITGB1.

1092. The method of alternative 1025, wherein said exosome expresses RAP1B.

1093. The method of alternative 1025, wherein said exosome expresses PRDX1.

1094. The method of alternative 1025, wherein said exosome expresses CFL1.

1095. The method of alternative 1025, wherein said exosome expresses MSN.

1096. The method of alternative 1025, wherein said exosome expresses PPIA.

1097. The method of alternative 1025, wherein said exosome expresses TPI1.

1098. The method of alternative 1025, wherein said exosome expresses VCP.

1099. The method of alternative 1025, wherein said exosome expresses ALB.

1100. The method of alternative 1025, wherein said exosome expresses ACLY.

1101. The method of alternative 1025, wherein said exosome expresses TUBA1C.

1102. The method of alternative 1025, wherein said exosome expresses TFRC.

1103. The method of alternative 1025, wherein said exosome expresses RAB14.

1104. The method of alternative 1025, wherein said exosome expresses HIST2H4A.

1105. The method of alternative 1025, wherein said exosome expresses GNB1.

1106. The method of alternative 1025, wherein said exosome expresses THBS1.

1107. The method of alternative 1025, wherein said exosome expresses RAN.

1108. The method of alternative 1025, wherein said exosome expresses RAB5A.

1109. The method of alternative 1025, wherein said exosome expresses PTGFRN.

1110. The method of alternative 1025, wherein said exosome expresses CCT5.

1111. The method of alternative 1025, wherein said exosome expresses CCT3.

1112. The method of alternative 1025, wherein said exosome expresses AHCY.

1113. The method of alternative 1025, wherein said exosome expresses UBA1.

1114. The method of alternative 1025, wherein said exosome expresses RAB5B.

1115. The method of alternative 1025, wherein said exosome expresses RAB1A.

1116. The method of alternative 1025, wherein said exosome expresses LAMP2.

1117. The method of alternative 1025, wherein said exosome expresses ITGA6.

1118. The method of alternative 1025, wherein said exosome expresses HIST1H4B.

1119. The method of alternative 1025, wherein said exosome expresses BSG.

1120. The method of alternative 1025, wherein said exosome expresses YWHAH.

1121. The method of alternative 1025, wherein said exosome expresses TUBA1A.

1122. The method of alternative 1025, wherein said exosome expresses MVP.

1123. The method of alternative 1025, wherein said exosome expresses TKT.

1124. The method of alternative 1025, wherein said exosome expresses TCP1.

1125. The method of alternative 1025, wherein said exosome expresses STOM.

1126. The method of alternative 1025, wherein said exosome expresses SLC16A1.

1127. The method of alternative 1025, wherein said exosome expresses RAB8A.

1128. The method of alternative 1025, wherein said exosome expresses MYH9.

1129. The method of alternative 1021, wherein said tumor derived microvesicles are concentrated and exposed to a fibroblast or fibroblast like-population.

1130. The method of alternative 1129, wherein said fibroblasts exposed to said tumor derived microvesicles are cultured for a period of time to allow said microvesicles to induce reprogramming of said fibroblasts.

1131. The method of alternative 1130, wherein said reprogramming is associated with induction of drug resistance.

1132. The method of alternative 1131, wherein said induction of drug resistance is associated with stimulation of anti-apoptotic genes.

1133. The method of alternative 1132, wherein said anti-apoptotic gene is bcl-2.

1134. The method of alternative 1132, wherein said anti-apoptotic gene is ucp2.

1135. The method of alternative 1132, wherein said anti-apoptotic gene is SOD2.

1136. The method of alternative 1132, wherein said anti-apoptotic gene is Inos.

1137. The method of alternative 1132, wherein said anti-apoptotic gene is IAP-3.

1138. The method of alternative 1132, wherein said anti-apoptotic gene is bcl-xl.

1139. The method of alternative 1132, wherein said anti-apoptotic gene is bcl-w.

1140. The method of alternative 1132, wherein said anti-apoptotic gene is BFL-M.

1141. The method of alternative 1132, wherein said anti-apoptotic gene is BRAG-1.

1142. The method of alternative 1132, wherein said anti-apoptotic gene is MCL-1.

1143. The method of alternative 1132, wherein said anti-apoptotic gene is A-1.

1144. The method of alternative 1129, wherein said fibroblast population is treated with a mitogenic agent to replicate a tumor cell.

1145. The method of alternative 1144, wherein said mitogenic agent is interleukin-1.

1146. The method of alternative 1144, wherein said mitogenic agent is interleukin-3.

1147. The method of alternative 1144, wherein said mitogenic agent is interleukin-6.

1148. The method of alternative 1144, wherein said mitogenic agent is interleukin-8.

1149. The method of alternative 1144, wherein said mitogenic agent is interleukin-9.

1150. The method of alternative 1144, wherein said mitogenic agent is interleukin-10.

1151. The method of alternative 1144, wherein said mitogenic agent is interleukin-11.

1152. The method of alternative 1144, wherein said mitogenic agent is interleukin-16.

1153. The method of alternative 1144, wherein said mitogenic agent is interleukin-17.

1154. The method of alternative 1144, wherein said mitogenic agent is interleukin-20.

1155. The method of alternative 1144, wherein said mitogenic agent is interleukin-22.

1156. The method of alternative 1144, wherein said mitogenic agent is interleukin-33.

1157. The method of alternative 1144, wherein said mitogenic agent is IGF-1.

1158. The method of alternative 1144, wherein said mitogenic agent is HGF-1.

1159. The method of alternative 1144, wherein said mitogenic agent is EGF-1.

1160. The method of alternative 1144, wherein said mitogenic agent is FGF-1.

1161. The method of alternative 1144, wherein said mitogenic agent is FGF-2.

1162. The method of alternative 1144, wherein said mitogenic agent is FGF-4.

1163. The method of alternative 1144, wherein said mitogenic agent is FGF-5.

1164. The method of alternative 1144, wherein said mitogenic agent is FGF-7.

1165. The method of alternative 1144, wherein said mitogenic agent is FGF-8.

1166. The method of alternative 1144, wherein said mitogenic agent is FGF-13

1167. The method of alternative 1144, wherein said mitogenic agent is PDGF-1.

1168. The method of alternative 1144, wherein said mitogenic agent is angiopoietin.

1169. The method of alternative 1144, wherein said mitogenic agent is transforming growth factor beta.

1170. The method of alternative 1129, wherein said fibroblast population is treated with a dedifferentiating agent in order to induce a phenotype similar to neoplasia.

1171. The method of alternative 1170, wherein said dedifferentiating agent is a histone deacetylase inhibitor.

1172. The method of alternative 1171, wherein said histone deacetylase inhibitor is MS-275.

1173. The method of alternative 1171, wherein said histone deacetylase inhibitor is CI-994.

1174. The method of alternative 1171, wherein said histone deacetylase inhibitor is MGCD-0103.

1175. The method of alternative 1171, wherein said histone deacetylase inhibitor is valproic acid.

1176. The method of alternative 1171, wherein said histone deacetylase inhibitor is sodium butyrate.

1177. The method of alternative 1171, wherein said histone deacetylase inhibitor is sodium phenylbutyrate.

1178. The method of alternative 1171, wherein said histone deacetylase inhibitor is romidepsin.

1179. The method of alternative 1171, wherein said histone deacetylase inhibitor is apicidin.

1180. The method of alternative 1171, wherein said histone deacetylase inhibitor is JNJ-26481585.

1181. The method of alternative 1171, wherein said histone deacetylase inhibitor is Vorinostat.

1182. The method of alternative 1171, wherein said histone deacetylase inhibitor is trichostatin.

1183. The method of alternative 1171, wherein said histone deacetylase inhibitor is panobinostat.

1184. The method of alternative 1129, wherein said fibroblast is reprogrammed to achieve a cancer, or cancer-stem cell like phenotype.

1185. The method of alternative 1184, wherein said reprogramming is achieved by culture with a DNA methyltransferase inhibitor.

1186. The method of alternative 1185, wherein said DNA methyltransferase inhibitory is a nucleic acid derivative.

1187. The method of alternative 1185, wherein said DNA methyltransferase inhibitory is decitabine.

1188. The method of alternative 1185, wherein said DNA methyltransferase inhibitory is 5-azacytabine.

1189. The method of alternative 1184, wherein said reprogramming is induced through suppression of the enzyme GSK-3.

1190. The method of alternative 1189, wherein said suppression of said enzyme GSK-3 is accomplished by treatment with lithium.

1191. The method of alternative 1190, wherein said lithium is administered at a concentration of 1 μg/ml to 1 mg/ml.

1192. The method of alternative 1190, wherein said lithium is administered at a concentration of 10 μg/ml to 100 ng/ml.

1193. The method of alternative 1190, wherein said lithium is administered at a concentration of 100 μg/ml to 10 ng/ml.

1194. The method of alternative 1184, wherein said reprogramming is induced through induction of expression of the gene OCT3/4.

1195. The method of alternative 1184, wherein said reprogramming is induced through induction of expression of the gene SOX2.

1196. The method of alternative 1184, wherein said reprogramming is induced through induction of expression of the gene KLF4.

1197. The method of alternative 1184, wherein said reprogramming is induced through induction of expression of the gene L-MYC.

1198. The method of alternative 1184, wherein said reprogramming is induced through induction of expression of the gene LIN28.

1199. The method of alternative 1184, wherein said reprogramming is induced through induction of expression of the gene BCL-xL.

1200. The method of alternative 1184, wherein said reprogramming is induced through induction of expression of the gene BCL-xL.

1201. The method of alternative 1184, wherein said reprogramming is induced through inhibition of p53 expression.

1202. The method of alternative 1201, wherein p53 expression is suppression of p53 activity.

1203. The method of alternative 1202, wherein said suppression of p53 activity is mediated through administration of a small molecule inhibitor of p53.

1204. The method of alternative 1202, wherein said suppression of p53 activity is mediated through administration of decoy oligonucleotides.

1205. The method of alternative 1202, wherein said suppression of p53 activity is mediated through administration of decoy peptides.

1206. The method of alternative 1202, wherein said suppression of p53 expression is achieved through induction of RNA interference targeting p53.

1207. The method of alternative 1206, wherein said induction of RNA interference targeting p53 is induced through administration of short interfering RNA.

1208. The method of alternative 1206, wherein said induction of RNA interference targeting p53 is induced through administration of short hairpin RNA.

1209. The method of alternative 1202, wherein said suppression of p53 expression is achieved through administration of antisense oligonucleotides targeting p53.

1210. The method of alternative 1209, wherein said antisense oligonucleotides induce cleavage of nucleic acids through activation of RNAse H.

1211. The method of alternative 1202, wherein said suppression of p53 expression is achieved through administration of ribozymes targeting p53.

1212. The method of alternative 1202, wherein said suppression of p53 expression is achieved through gene editing.

1213. The method of alternative 1142, wherein said reprogramming is induced by culture of said cells in a liquid media containing ascorbic acid.

1214. The method of alternative 1142, wherein said reprogramming is induced by culture of said cells in a liquid media containing transferrin.

1215. The method of alternative 1142, wherein said reprogramming is induced by culture of said cells in a liquid media containing sodium bicarbonate.

1216. The method of alternative 1142, wherein said reprogramming is induced by culture of said cells in a liquid media containing insulin.

1217. The method of alternative 1142, wherein said reprogramming is induced by culture of said cells in a liquid media containing sodium selenite.

1218. The method of alternative 1142, wherein said reprogramming is induced by culture of said cells in a hypoxic environment.

1219. The method of alternative 1208, wherein said hypoxic environment comprises of oxygen levels low enough to induce activation of hypoxia inducible factor (HIF)-1.

1220. The method of alternative 1218, wherein said hypoxic environment is culture of said cells in an environment less than 21% oxygen.

1221. The method of alternative 1218, wherein said hypoxic environment is culture of said cells in an environment less than 15% oxygen.

1222. The method of alternative 1218, wherein said hypoxic environment is culture of said cells in an environment less than 10% oxygen.

1223. The method of alternative 1218, wherein said hypoxic environment is culture of said cells in an environment containing approximately 5% oxygen.

1224. The method of alternative 1184, wherein said reprogramming is induced by culture of cells in a liquid media containing a MAP Kinase inhibitor.

1225. The method of alternative 1224, wherein said MAP kinase inhibitor is PD0325901/

1226. The method of alternative 1184, wherein said reprogramming is induced by culture of cells in a liquid media containing SB431542.

1227. The method of alternative 1184, wherein said reprogramming is induced by culture of cells in a liquid media containing CHIR99021.

1228. The method of alternative 1184, wherein said reprogramming is induced by culture of cells in a liquid media containing Y-27632.

1229. The method of alternative 1184, wherein said reprogramming is induced by culture of cells in a liquid media containing Y-thiazovivin.

1230. The method of alternative 1184, wherein said reprogramming is induced by culture of cells in a liquid media containing FGF-1.

1231. The method of alternative 1184, wherein said reprogramming is induced by culture of cells in a liquid media containing FGF-2.

1232. The method of alternative 1184, wherein said reprogramming is induced by culture of cells in a liquid media containing FGF-5.

1233. The method of alternative 1184, wherein said reprogramming is induced by culture of cells in a liquid media containing sodium borate.

1234. The method of alternative 1184, wherein said reprogramming is induced by culture of cells in a liquid media containing erythropoietin (EPO).

1235. The method of alternative 1184, wherein said reprogramming is induced by culture of cells in a liquid media containing interleukin-3.

1236. The method of alternative 1184, wherein said reprogramming is induced by culture of cells in a liquid media containing interleukin-6.

1237. The method of alternative 1184, wherein said reprogramming is induced by culture of cells in a liquid media containing interleukin-8.

1238. The method of alternative 1184, wherein said reprogramming is induced by culture of cells in a liquid media containing interleukin-10.

1239. The method of alternative 1184, wherein said reprogramming is induced by culture of cells in a liquid media containing interleukin-18.

1240. The method of alternative 1184, wherein said reprogramming is induced by culture of cells in a liquid media containing interleukin-20.

1241. The method of alternative 1184, wherein said reprogramming is induced by culture of cells in a liquid media containing interleukin-25.

1242. The method of alternative 1184, wherein said reprogramming is induced by culture of cells in a liquid media containing insulin-like growth factor-1 (IGF-1).

1243. The method of alternative 1184, wherein said reprogramming is induced by culture of cells in a liquid media containing dexamethasone.

1244. The method of alternative 1184, wherein said reprogramming is induced by culture of cells in a liquid media containing holo-transferrin.

1245. The method of alternative 1184, wherein said reprogramming is induced by culture of cells in a liquid media containing amino acids selected from a group comprising of Glycine, L-histidine, L-isoleucine, L-methionine, L-phenylalanine, L-proline, L-hydroxyproline, L-serine, L-threonine, L-tryptophan, and L-tyrosine, L-valine.

1246. The method of alternative 1184, wherein said reprogramming is induced by culture of cells in a liquid media containing vitamins and/or antioxidants selected from a group comprising of thiamine, reduced glutathione, ascorbic acid and 2-PO.sub.4.

1247. The method of alternative 1184, wherein said reprogramming is induced by culture of cells in a liquid media containing trace elements selected from a group comprising of: Ag.sup.+, Al.sup.3+, Ba.sup.2+, Cd.sup.2+, Co.sup.2+, Cr.sup.3+, Ge.sup.4+, Se.sup.4+, Br.sup.−, I.sup.−, F.sup.−, Mn.sup.2+, Si.sup.4+, V.sup.5+, MO.sup.6+, Ni.sup.2+, Rb.sup.+, Sn.sup.2+, and Zr.sup.4+.

1248. The method of alternative 1184, wherein said reprogramming is induced by culture of cells in a liquid media containing cytoplasm of an undifferentiated cell.

1249. The method of alternative 1248, wherein said cell being reprogrammed has its membrane temporarily permeabilized.

1250. The method of 1249, wherein said temporary permeabilization allows for entry of cytoplasm of undifferentiated cell into cytoplasm of said cell to be reprogrammed.

1251. The method of alternative 1248, wherein said undifferentiated cell is syngeneic with the cell whose reprogramming is desired.

1252. The method of alternative 1248, wherein said undifferentiated cell is allogeneic with the cell whose reprogramming is desired.

1253. The method of alternative 1248, wherein said undifferentiated cell is xenogeneic with the cell whose reprogramming is desired.

1254. The method of alternative 1249, wherein said permeabilization is mediated by electroporation.

1255. The method of alternative 1249, wherein said permeabilization is mediated by Streptolysin O treatment.

1256. The method of alternative 1249, wherein said permeabilization is mediated by transient treatment with complement membrane attack complex.

1257. The method of alternative 1249, wherein said permeabilization is mediated by transient treatment with perforin.

1258. The method of alternative 1249, wherein said permeabilization is mediated by transient treatment with granzyme.

1259. The method of alternative 1248, wherein said undifferentiated cell is an oocyte.

1260. The method of alternative 1259, wherein said oocyte is programmed to be at G0/G1 of cell cycle.

1261. The method of alternative 1260, wherein said programming to be at G0/G1 of cell cycle is accomplished by exposure to mitomycin C.

1262. The method of alternative 1260, wherein said programming to be at G0/G1 of cell cycle is accomplished by exposure to serum starvation.

1263. The method of alternative 1248, wherein said undifferentiated cell is an inducible pluripotent stem cell.

1264. The method of alternative 1248, wherein said undifferentiated cell is a parthenogenic derived stem cell.

1265. The method of alternative 1248, wherein said undifferentiated cell is an embryonic stem cell.

1266. The method of alternative 1248, wherein said undifferentiated cell is a somatic cell nuclear transfer derived stem cell.

1268. The method of alternative 1248, wherein said undifferentiated cell is a cytoplasmically reprogrammed stem cell.

1269. The method of alternative 1248, wherein said undifferentiated cell is a cell obtained by fusion of an adult cell with a pluripotent stem cell.

1270. The method of alternative 1269, wherein said fusion is accomplished by the use of polyethylene glycol.

1271. The method of alternative 1269, wherein said fusion is accomplished by the use of electrically mediated fusion.

1272. The method of alternative 1002, wherein said cancer treatment is an immunotherapy.

1273. The method of alternative 1272, wherein said chemotherapy is selected from a group comprising of: methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbazine, etoposides, camptothecins, bleomycin, doxorubicin, idarubicin, daunorubicin, dactinomycin, plicamycin, mitoxantrone, asparaginase, vinblastine, vincristine, vinorelbine, paclitaxel, and docetaxel, doxorubicin, epirubicin, 5-fluorouracil, taxanes such as docetaxel and paclitaxel, leucovorin, levamisole, irinotecan, estramustine, etoposide, nitrosoureas such as carmustine and lomustine, vinca alkaloids, platinum compounds, mitomycin, gemcitabine, hexamethylmelamine, topotecan, tyrosine kinase inhibitors, tyrphostins, STI-571 or Gleevec.™. (imatinib mesylate), herbimycin A, genistein, erbstatin, and lavendustin A. taxol, mercaptopurine, thioguanine, hydroxyurea, cytarabine, cyclophosphamide, ifosfamide, nitrosoureas, cisplatin, carboplatin, mitomycin, dacarbazine, procarbazine, etoposides, camptothecins, bleomycin, doxorubicin, idarubicin, daunorubicin, dactinomycin, plicamycin, mitoxantrone, asparaginase, vinblastine, vincristine, vinorelbine, paclitaxel, and docetaxel, doxorubicin, epirubicin, 5-fluorouracil, taxanes such as docetaxel and paclitaxel, leucovorin, levamisole, irinotecan, estramustine, etoposide, nitrosoureas such as carmustine and lomustine, vinca alkaloids, platinum compounds, mitomycin, gemcitabine, hexamethylmelamine, topotecan, tyrosine kinase inhibitors, tyrphostinsherbimycin A, genistein, erbstatin, and lavendustin ABCNU, irinotecan, camptothecins, bleomycin, doxorubicin, idarubicin, daunorubicin, dactinomycin, plicamycin, mitoxantrone, asparaginase, vinblastine, vincristine, vinorelbine, paclitaxel, and docetaxel. In a preferred embodiment, the anti-cancer agent can be, but is not limited to, a drug listed: Alkylating agents Nitrogen mustards: Cyclophosphamide Ifosfamide Trofosfamide Chlorambucil Nitrosoureas: Carmustine (BCNU) Lomustine (CCNU) Alkylsulphonates: Busulfan Treosulfan Triazenes: Dacarbazine Platinum containing Cisplatin compounds: Carboplatin Aroplatin Oxaliplatin Plant Alkaloids Vinca alkaloids: Vincristine Vinblastine Vindesine Vinorelbine Taxoids: Paclitaxel Docetaxel DNA Topoisomerase Inhibitors Epipodophyllins: Etoposide Teniposide Topotecan 9-aminocamptothecin Camptothecin Crisnatol mitomycins: Mitomycin C Antimetabolites Anti-folates: DHFR inhibitors: Methotrexate Trimetrexate IMP dehydrogenase Mycophenolic acid Inhibitors: Tiazofurin Ribavirin EICAR Ribonucleotide reductase Hydroxyurea Inhibitors: Deferoxamine Pyrimidine analogs: Uracil analogs: 5-Fluorouracil Floxuridine Doxifluridine Raltitrexed Cytosine analogs: Cytarabine (ara C) Cytosine arabinoside Fludarabine Purine analogs: Mercaptopurine Thioguanine DNA Antimetabolites: 3-HP 2′-deoxy-5-fluorouridine 5-HP alpha-TGDR aphidicolin glycinate ara-C 5-aza-2′-deoxycytidine beta-TGDR cyclocytidine guanazole inosine glycidaldehyde macebecin II pyrazoloimidazole Hormonal therapies: Receptor antagonists: Anti-estrogen: Tamoxifen Raloxifene Megestrol LHRH agonists: Goserelin Leuprolide acetate Anti-androgens: Flutamide Bicalutamide Retinoids/Deltoids Cis-retinoic acid Vitamin A derivative: All-trans retinoic acid (ATRA-IV) Vitamin D3 analogs: EB 1089 CB 1093 KH 1060 Photodynamic therapies: Verteporfin (BPD-MA) Phthalocyanine Photosensitizer Pc4 Demethoxy-hypocrellin A (2BA-2-DMHA) Cytokines: Interferon-.alpha. Interferon-.gamma. Tumor necrosis factor Angiogenesis Inhibitors: Angiostatin (plasminogen fragment) antiangiogenic antithrombin III Angiozyme ABT-627 Bay 12-9566 Benefin Bevacizumab BMS-275291 cartilage-derived inhibitor (CDI) CAI CD59 complement fragment CEP-7055 Col 3 Combretastatin A-4 Endostatin (collagen XVIII fragment) Fibronectin fragment Gro-beta Halofuginone Heparanases Heparin hexasaccharide fragment HMV833 Human chorionic gonadotropin (hCG) IM-862 Interferon alpha/beta/gamma Interferon inducible protein (IP-10) Interleukin-12 Kringle 5 (plasminogen fragment) Marimastat Metalloproteinase inhibitors (TIMPs) 2-Methoxyestradiol MMI 270 (CGS 27023A) MoAb IMC-1C11 Neovastat NM-3 Panzem PI-88 Placental ribonuclease inhibitor Plasminogen activator inhibitor Platelet factor-4 (PF4) Prinomastat Prolactin 16 kD fragment Proliferin-related protein (PRP) PTK 787/ZK 222594 Retinoids Solimastat Squalamine SS 3304 SU 5416 SU6668 SU11248 Tetrahydrocortisol-S tetrathiomolybdate thalidomide Thrombospondin-1 (TSP-1) TNP-470 Transforming growth factor-beta (TGF-b) Vasculostatin Vasostatin (calreticulin fragment) ZD6126 ZD 6474 farnesyl transferase inhibitors (FTI) bisphosphonates Antimitotic agents: allocolchicine Halichondrin B colchicine colchicine derivative dolastatin 10 maytansine rhizoxin thiocolchicine trityl cysteine Others: Isoprenylation inhibitors: Dopaminergic neurotoxins: 1-methyl-4-phenylpyridinium ion Cell cycle inhibitors: Staurosporine Actinomycins: Actinomycin D Dactinomycin Bleomycins: Bleomycin A2 Bleomycin B2 Peplomycin Anthracyclines: Daunorubicin Doxorubicin (adriamycin) Idarubicin Epirubicin Pirarubicin Zorubicin Mitoxantrone MDR inhibitors: Verapamil Ca.sup.2+ATPase inhibitors: Thapsigargin.

1274. The method of alternative 1002, wherein ability of drugs to augment immunogenicity of tumor cells is assessed by first treating said tumor cells with a variety of test drugs or compounds, and subsequently assessing said tumors for markers of immunogenicity.

1275. The method of alternative 1274, wherein said marker of immunogenicity is HLA-A.

1276. The method of alternative 1274, wherein said marker of immunogenicity is HLA-B.

1277. The method of alternative 1274, wherein said marker of immunogenicity is HLA-C.

1278. The method of alternative 1274, wherein said marker of immunogenicity is HLA-DR.

1279. The method of alternative 1274, wherein said marker of immunogenicity is transporter associated protein-1 (TAP-1).

1280. The method of alternative 1274, wherein said marker of immunogenicity is CD40.

1281. The method of alternative 1274, wherein said marker of immunogenicity is CD80.

1282. The method of alternative 1274, wherein said marker of immunogenicity is CD86.

1283. The method of alternative 1274, wherein said marker of immunogenicity is interleukin-1 production.

1284. The method of alternative 1274, wherein said marker of immunogenicity is interleukin-6 production.

1285. The method of alternative 1274, wherein said marker of immunogenicity is interleukin-23 production.

1286. The method of alternative 1274, wherein said marker of immunogenicity is interleukin-17 production.

1287. The method of alternative 1274, wherein said marker of immunogenicity is interleukin-1 production.

### REFERENCES

1. Ugel, S., et al., Targeting tumor vasculature: expanding the potential of DNA cancer vaccines. Cancer Immunol Immunother, 2015. 64 (10): p. 1339-48.
2. Li, L., et al., Developing a clinical development paradigm for translation of a mammaglobin—A DNA vaccine. Immunotherapy, 2015: p. 1-3.
3. Tiriveedhi, V., et al., Safety and preliminary evidence of biologic efficacy of a mammaglobin—a DNA vaccine in patients with stable metastatic breast cancer. Clin Cancer Res, 2014. 20 (23): p. 5964-75.
4. Heller, R. and L. C. Heller, Gene electrotransfer clinical trials. Adv Genet, 2015. 89: p. 235-62.
5. Butterfield, L. H., et al., Alpha fetoprotein DNA prime and adenovirus boost immunization of two hepatocellular cancer patients. J Transl Med, 2014. 12: p. 86.
6. Sharpe, M., et al., Protection of mice from H5N1 influenza challenge by prophylactic DNA vaccination using particle mediated epidermal delivery. Vaccine, 2007. 25 (34): p. 6392-8.
7. Loudon, P. T., et al., GM-CSF increases mucosal and systemic immunogenicity of an H1N1 influenza DNA vaccine administered into the epidermis of non-human primates. PLOS One, 2010. 5 (6): p. e11021.
8. Jones, S., et al., DNA vaccination protects against an influenza challenge in a double-blind randomised placebo-controlled phase 1b clinical trial. Vaccine, 2009. 27 (18): p. 2506-12.
9. Shah, M. A., et al., DNA Mediated Vaccines Delivery Through Nanoparticles. J Nanosci Nanotechnol, 2015. 15 (1): p. 41-53.
10. Mpendo, J., et al., A Phase I Double Blind, Placebo-Controlled, Randomized Study of the Safety and Immunogenicity of Electroporated HIV DNA with or without Interleukin 12 in Prime-Boost Combinations with an Ad35 HIV Vaccine in Healthy HIV-Seronegative African Adults. PLOS One, 2015. 10 (8): p. e0134287.
11. Keane-Myers, A. M., et al., DNA electroporation of multi-agent vaccines conferring protection against select agent challenge: TriGrid delivery system. Methods Mol Biol, 2014. 1121: p. 325-36.
12. Hooper, J. W., et al., A Phase 1 clinical trial of Hantaan virus and Puumala virus M-segment DNA vaccines for haemorrhagic fever with renal syndrome delivered by intramuscular electroporation. Clin Microbiol Infect, 2014. 20 Suppl 5: p. 110-7.
13. van Furth, R. and Z. A. Cohn, The origin and kinetics of mononuclear phagocytes. J Exp Med, 1968. 128 (3): p. 415-35.
14. Wynn, T. A., A. Chawla, and J. W. Pollard, Macrophage biology in development, homeostasis and disease. Nature, 2013. 496 (7446): p. 445-55.
15. Smith, T. D., et al., Harnessing macrophage plasticity for tissue regeneration. Adv Drug Deliv Rev, 2017.
16. Vannella, K. M. and T. A. Wynn, Mechanisms of Organ Injury and Repair by Macrophages. Annu Rev Physiol, 2017. 79: p. 593-617.
17. Boddupalli, A., L. Zhu, and K. M. Bratlie, Methods for Implant Acceptance and Wound Healing: Material Selection and Implant Location Modulate Macrophage and Fibroblast Phenotypes. Adv Healthc Mater, 2016. 5 (20): p. 2575-2594.
18. Snyder, R. J., et al., Macrophages: A review of their role in wound healing and their therapeutic use. Wound Repair Regen, 2016. 24 (4): p. 613-29.
19. Gombozhapova, A., et al., Macrophage activation and polarization in post-infarction cardiac remodeling. J Biomed Sci, 2017. 24 (1): p. 13.
20. Hu, Y., et al., Class A scavenger receptor attenuates myocardial infarction-induced cardiomyocyte necrosis through suppressing M1 macrophage subset polarization. Basic Res Cardiol, 2011. 106 (6): p. 1311-28.
21. Ma, Y., et al., Matrix metalloproteinase-28 deletion exacerbates cardiac dysfunction and rupture after myocardial infarction in mice by inhibiting M2 macrophage activation. Circ Res, 2013. 112 (4): p. 675-88.
22. Lee, C. W., et al., Macrophage heterogeneity of culprit coronary plaques in patients with acute myocardial infarction or stable angina. Am J Clin Pathol, 2013. 139 (3): p. 317-22.
23. Yan, X., et al., Temporal dynamics of cardiac immune cell accumulation following acute myocardial infarction. J Mol Cell Cardiol, 2013. 62: p. 24-35.
24. Fernandez-Velasco, M., S. Gonzalez-Ramos, and L. Bosca, Involvement of monocytes/macrophages as key factors in the development and progression of cardiovascular diseases. Biochem J, 2014. 458 (2): p. 187-93.
25. de Couto, G., et al., Macrophages mediate cardioprotective cellular postconditioning in acute myocardial infarction. J Clin Invest, 2015. 125 (8): p. 3147-62.
26. Guiteras, R., M. Flaquer, and J. M. Cruzado, Macrophage in chronic kidney disease. Clin Kidney J, 2016. 9 (6): p. 765-771.
27. Meng, X. M., et al., Macrophage Phenotype in Kidney Injury and Repair. Kidney Dis (Basel), 2015. 1 (2): p. 138-46.
28. Yamamoto, S., et al., Atherosclerosis following renal injury is ameliorated by pioglitazone and losartan via macrophage phenotype. Atherosclerosis, 2015. 242 (1): p. 56-64.
29. Li, C., et al., Enhanced M1 and Impaired M2 Macrophage Polarization and Reduced Mitochondrial Biogenesis via Inhibition of AMP Kinase in Chronic Kidney Disease. Cell Physiol Biochem, 2015. 36 (1): p. 358-72.
30. Sun, Y. Y., et al., Macrophage Phenotype in Liver Injury and Repair. Scand J Immunol, 2017. 85 (3): p. 166-174.
31. Gratchev, A., et al., Mphi1 and Mphi2 can be re-polarized by Th2 or Th1 cytokines, respectively, and respond to exogenous danger signals. Immunobiology, 2006. 211 (6-8): p. 473-86.
32. Mills, C. D., M1 and M2 Macrophages: Oracles of Health and Disease. Crit Rev Immunol, 2012. 32 (6): p. 463-88.
33. Mills, C. D. and K. Ley, M1 and M2 macrophages: the chicken and the egg of immunity. J Innate Immun, 2014. 6 (6): p. 716-26.
34. Alsaid, H., et al., Non invasive imaging assessment of the biodistribution of GSK2849330, an ADCC and CDC optimized anti HER3 mAb, and its role in tumor macrophage recruitment in human tumor-bearing mice. PLOS One, 2017. 12 (4): p. e0176075.
35. Josephs, D. H., et al., Anti-Folate Receptor-alpha IgE but not IgG Recruits Macrophages to Attack Tumors via TNFalpha/MCP-1 Signaling. Cancer Res, 2017. 77 (5): p. 1127-1141.
36. Velmurugan, R., et al., Macrophage-Mediated Trogocytosis Leads to Death of Antibody-Opsonized Tumor Cells. Mol Cancer Ther, 2016. 15 (8): p. 1879-89.
37. Gul, N. and M. van Egmond, Antibody-Dependent Phagocytosis of Tumor Cells by Macrophages: A Potent Effector Mechanism of Monoclonal Antibody Therapy of Cancer. Cancer Res, 2015. 75 (23): p. 5008-13.
38. Church, A. K., et al., Anti-CD20 monoclonal antibody-dependent phagocytosis of chronic lymphocytic leukaemia cells by autologous macrophages. Clin Exp Immunol, 2016. 183 (1): p. 90-101.
39. Shi, Y., et al., Trastuzumab triggers phagocytic killing of high HER2 cancer cells in vitro and in vivo by interaction with Fcgamma receptors on macrophages. J Immunol, 2015. 194 (9): p. 4379-86.
40. Weiskopf, K. and I. L. Weissman, Macrophages are critical effectors of antibody therapies for cancer. MAbs, 2015. 7 (2): p. 303-10.
41. Oflazoglu, E., et al., Macrophages contribute to the antitumor activity of the anti-CD30 antibody SGN-30. Blood, 2007. 110 (13): p. 4370-2.
42. Osman, R., et al., Calreticulin Release at an Early Stage of Death Modulates the Clearance by Macrophages of Apoptotic Cells. Front Immunol, 2017. 8: p. 1034.
43. Feng, M., et al., Macrophages eat cancer cells using their own calreticulin as a guide: roles of TLR and Btk. Proc Natl Acad Sci USA, 2015. 112 (7): p. 2145-50.
44. Chao, M. P., et al., Calreticulin is the dominant pro-phagocytic signal on multiple human cancers and is counterbalanced by CD47. Sci Transl Med, 2010. 2 (63): p. 63ra94.
45. Murata, Y., et al., The CD47-SIRPalpha signalling system: its physiological roles and therapeutic application. J Biochem, 2014. 155 (6): p. 335-44.
46. Roberts, D. D., S. Kaur, and D. R. Soto-Pantoja, Therapeutic targeting of the thrombospondin-1 receptor CD47 to treat liver cancer. J Cell Commun Signal, 2015. 9 (1): p. 101-2.
47. Liu, J., et al., Pre-Clinical Development of a Humanized Anti-CD47 Antibody with Anti-Cancer Therapeutic Potential. PLOS One, 2015. 10 (9): p. e0137345.
48. Weiskopf, K., et al., CD47-blocking immunotherapies stimulate macrophage-mediated destruction of small-cell lung cancer. J Clin Invest, 2016. 126 (7): p. 2610-20.
49. Weiskopf, K., et al., Eradication of Canine Diffuse Large B-Cell Lymphoma in a Murine Xenograft Model with CD47 Blockade and Anti-CD20. Cancer Immunol Res, 2016. 4 (12): p. 1072-1087.
50. Zeng, D., et al., A fully human anti-CD47 blocking antibody with therapeutic potential for cancer. Oncotarget, 2016. 7 (50): p. 83040-83050.
51. Liljefors, M., et al., Influence of varying doses of granulocyte-macrophage colony-stimulating factor on pharmacokinetics and antibody-dependent cellular cytotoxicity. Cancer Immunol Immunother, 2008. 57 (3): p. 379-88.
52. Tarr, P. E., Granulocyte-macrophage colony-stimulating factor and the immune system. Med Oncol, 1996. 13 (3): p. 133-40.
53. Ragnhammar, P., et al., Cytotoxicity of white blood cells activated by granulocyte-colony-stimulating factor, granulocyte/macrophage-colony-stimulating factor and macrophage-colony-stimulating factor against tumor cells in the presence of various monoclonal antibodies. Cancer Immunol Immunother, 1994. 39 (4): p. 254-62.
54. Ragnhammar, P., Anti-tumoral effect of GM-CSF with or without cytokines and monoclonal antibodies in solid tumors. Med Oncol, 1996. 13 (3): p. 167-76.
55. Lin, E. Y., et al., Colony-stimulating factor 1 promotes progression of mammary tumors to malignancy. J Exp Med, 2001. 193 (6): p. 727-40.
56. Aharinejad, S., et al., Colony-stimulating factor-1 blockade by antisense oligonucleotides and small interfering RNAs suppresses growth of human mammary tumor xenografts in mice. Cancer Res, 2004. 64 (15): p. 5378-84.
57. Lin, E. Y., et al., Macrophages regulate the angiogenic switch in a mouse model of breast cancer. Cancer Res, 2006. 66 (23): p. 11238-46.
58. Lin, E. Y. and J. W. Pollard, Tumor-associated macrophages press the angiogenic switch in breast cancer. Cancer Res, 2007. 67 (11): p. 5064-6.
59. Zhang, W. J., et al., Hypoxia-inducible factor-1 alpha Correlates with Tumor-Associated Macrophages Infiltration, Influences Survival of Gastric Cancer Patients. J Cancer, 2017. 8 (10): p. 1818-1825.
60. Yuan, X., et al., Prognostic significance of tumor-associated macrophages in ovarian cancer: A meta-analysis. Gynecol Oncol, 2017. 147 (1): p. 181-187.
61. Ma, C., et al., CD163-positive cancer cells are potentially associated with high malignant potential in clear cell renal cell carcinoma. Med Mol Morphol, 2017.
62. Shi, Y., et al., Tumour-associated macrophages secrete pleiotrophin to promote PTPRZ1 signalling in glioblastoma stem cells for tumour growth. Nat Commun, 2017. 8: p. 15080.
63. Zhao, X., et al., Prognostic significance of tumor-associated macrophages in breast cancer: a meta-analysis of the literature. Oncotarget, 2017. 8 (18): p. 30576-30586.
64. Pearce, O. M., et al., Inverse hormesis of cancer growth mediated by narrow ranges of tumor-directed antibodies. Proc Natl Acad Sci USA, 2014. 111 (16): p. 5998-6003.
65. Pander, J., et al., Activation of tumor-promoting type 2 macrophages by EGFR-targeting antibody cetuximab. Clin Cancer Res, 2011. 17 (17): p. 5668-73.
66. Clynes, R. A., et al., Inhibitory Fc receptors modulate in vivo cytotoxicity against tumor targets. Nat Med, 2000. 6 (4): p. 443-6.
67. Pricop, L., et al., Differential modulation of stimulatory and inhibitory Fc gamma receptors on human monocytes by Th1 and Th2 cytokines. J Immunol, 2001. 166 (1): p. 531-7.
68. Tridandapani, S., et al., Regulated expression and inhibitory function of Fcgamma RIIb in human monocytic cells. J Biol Chem, 2002. 277 (7): p. 5082-9.
69. Joshi, T., et al., Molecular analysis of expression and function of hFcgammaRIIbl and b2 isoforms in myeloid cells. Mol Immunol, 2006. 43 (7): p. 839-50.
70. Wijngaarden, S., et al., A shift in the balance of inhibitory and activating Fcgamma receptors on monocytes toward the inhibitory Fcgamma receptor IIb is associated with prevention of monocyte activation in rheumatoid arthritis. Arthritis Rheum, 2004. 50 (12): p. 3878-87.
71. Butchar, J. P., et al., Reciprocal regulation of activating and inhibitory Fc{gamma} receptors by TLR7/8 activation: implications for tumor immunotherapy. Clin Cancer Res, 2010. 16 (7): p. 2065-75.
72. Fatehchand, K., et al., Toll-like Receptor 4 Ligands Down-regulate Fcgamma Receptor IIb (FcgammaRIIb) via MARCH3 Protein-mediated Ubiquitination. J Biol Chem, 2016. 291 (8): p. 3895-904.
73. Ghochikyan, A., et al., Targeting TLR-4 with a novel pharmaceutical grade plant derived agonist, Immunomax®, as a therapeutic strategy for metastatic breast cancer. J Transl Med, 2014. 12: p. 322.
74. Oronsky, B., et al., RRx-001: a systemically non-toxic M2-to-M1 macrophage stimulating and prosensitizing agent in Phase II clinical trials. Expert Opin Investig Drugs, 2017. 26 (1): p. 109-119.
75. Lee, C., et al., Melittin suppresses tumor progression by regulating tumor-associated macrophages in a Lewis lung carcinoma mouse model. Oncotarget, 2017. 8 (33): p. 54951-54965.
76. Zhang, Q., et al., Clinical Effects of CpG-Based Treatment on the Efficacy of Hepatocellular Carcinoma by Skewing Polarization Toward M1 Macrophage from M2. Cancer Biother Radiopharm, 2017. 32 (6): p. 215-219.
77. Sato, T., et al., Intrapulmonary Delivery of CpG Microparticles Eliminates Lung Tumors. Mol Cancer Ther, 2015. 14 (10): p. 2198-205.
78. Chiang, C. F., et al., Metformin-treated cancer cells modulate macrophage polarization through AMPK-NF-kappaB signaling. Oncotarget, 2017. 8 (13): p. 20706-20718.
79. Kang, H., et al., Puerarin inhibits M2 polarization and metastasis of tumor-associated macrophages from NSCLC xenograft model via inactivating MEK/ERK 1/2 pathway. Int J Oncol, 2017. 50 (2): p. 545-554.
80. Jia, X., et al., Emodin suppresses pulmonary metastasis of breast cancer accompanied with decreased macrophage recruitment and M2 polarization in the lungs. Breast Cancer Res Treat, 2014. 148 (2): p. 291-302.
81. Xue, N., et al., Chlorogenic acid inhibits glioblastoma growth through repolarizating macrophage from M2 to M1 phenotype. Sci Rep, 2017. 7: p. 39011.
82. Sloan, E. K., et al., The sympathetic nervous system induces a metastatic switch in primary breast cancer. Cancer Res, 2010. 70 (18): p. 7042-52.
83. Liu, B., et al., Polarization of M1 tumor associated macrophage promoted by the activation of TLR3 signal pathway. Asian Pac J Trop Med, 2016. 9 (5): p. 484-8.
84. Liu, Q., et al., NMAAP1 Expressed in BCG-Activated Macrophage Promotes M1 Macrophage Polarization. Mol Cells, 2015. 38 (10): p. 886-94.
85. Liu, Y., et al., Polysaccharide Agaricus blazei Murill stimulates myeloid derived suppressor cell differentiation from M2 to M1 type, which mediates inhibition of tumour immune-evasion via the Toll-like receptor 2 pathway. Immunology, 2015. 146 (3): p. 379-91.
86. Yang, Y., et al., LPS converts Gr-1 (+) CD115 (+) myeloid-derived suppressor cells from M2 to M1 via P38 MAPK. Exp Cell Res, 2013. 319 (12): p. 1774-83.
87. Sanchez-Quesada, C., A. Lopez-Biedma, and J. J. Gaforio, Maslinic Acid enhances signals for the recruitment of macrophages and their differentiation to m1 state. Evid Based Complement Alternat Med, 2015. 2015: p. 654721.
88. Dominguez-Soto, A., et al., Intravenous immunoglobulin promotes antitumor responses by modulating macrophage polarization. J Immunol, 2014. 193 (10): p. 5181-9.
89. Yin, Y., et al., Phosphatidylserine-targeting antibody induces M1 macrophage polarization and promotes myeloid-derived suppressor cell differentiation. Cancer Immunol Res, 2013. 1 (4): p. 256-68.
90. Deng, R., et al., Dimethyl Sulfoxide Suppresses Mouse 4T1 Breast Cancer Growth by Modulating Tumor-Associated Macrophage Differentiation. J Breast Cancer, 2014. 17 (1): p. 25-32.
91. Mitsuhashi, A., et al., Surfactant protein A suppresses lung cancer progression by regulating the polarization of tumor-associated macrophages. Am J Pathol, 2013. 182 (5): p. 1843-53.
92. Coscia, M., et al., Zoledronic acid repolarizes tumour-associated macrophages and inhibits mammary carcinogenesis by targeting the mevalonate pathway. J Cell Mol Med, 2010. 14 (12): p. 2803-15.
93. Eriksson, F., et al., Tumor-specific bacteriophages induce tumor destruction through activation of tumor-associated macrophages. J Immunol, 2009. 182 (5): p. 3105-11.
94. Steinman, R. M. and Z. A. Cohn, Identification of a novel cell type in peripheral lymphoid organs of mice. I. Morphology, quantitation, tissue distribution. J Exp Med, 1973. 137 (5): p. 1142-62.
95. Banchereau, J. and R. M. Steinman, Dendritic cells and the control of immunity. Nature, 1998. 392 (6673): p. 245-52.
96. Trombetta, E. S. and I. Mellman, Cell biology of antigen processing in vitro and in vivo. Annu Rev Immunol, 2005. 23: p. 975-1028.
97. Itano, A. A. and M. K. Jenkins, Antigen presentation to naive CD4 T cells in the lymph node. Nat Immunol, 2003. 4 (8): p. 733-9.
98. Tjoa, B. A., et al., Evaluation of phase I/II clinical trials in prostate cancer with dendritic cells and PSMA peptides. Prostate, 1998. 36 (1): p. 39-44.
99. Murphy, G. P., et al., Infusion of dendritic cells pulsed with HLA-A2-specific prostate-specific membrane antigen peptides: a phase II prostate cancer vaccine trial involving patients with hormone-refractory metastatic disease. Prostate, 1999. 38 (1): p. 73-8.
100. Lodge, P. A., et al., Dendritic cell-based immunotherapy of prostate cancer: immune monitoring of a phase II clinical trial. Cancer Res, 2000. 60 (4): p. 829-33.
101. Burch, P. A., et al., Priming tissue-specific cellular immunity in a phase I trial of autologous dendritic cells for prostate cancer. Clin Cancer Res, 2000. 6 (6): p. 2175-82.
102. Nestle, F. O., et al., Vaccination of melanoma patients with peptide- or tumor lysate-pulsed dendritic cells. Nat Med, 1998. 4 (3): p. 328-32.
103. Chakraborty, N. G., et al., Immunization with a tumor-cell-lysate-loaded autologous-antigen-presenting-cell-based vaccine in melanoma. Cancer Immunol Immunother, 1998. 47 (1): p. 58-64.
104. Wang, F., et al., Phase I trial of a MART-1 peptide vaccine with incomplete Freund's adjuvant for resected high-risk melanoma. Clin Cancer Res, 1999. 5 (10): p. 2756-65.
105. Thurner, B., et al., Vaccination with mage-3A1 peptide-pulsed mature, monocyte-derived dendritic cells expands specific cytotoxic T cells and induces regression of some metastases in advanced stage IV melanoma. J Exp Med, 1999. 190 (11): p. 1669-78.
106. Thomas, R., et al., Immature human monocyte-derived dendritic cells migrate rapidly to draining lymph nodes after intradermal injection for melanoma immunotherapy. Melanoma Res, 1999. 9 (5): p. 474-81.
107. Mackensen, A., et al., Phase I study in melanoma patients of a vaccine with peptide-pulsed dendritic cells generated in vitro from CD34 (+) hematopoietic progenitor cells. Int J Cancer, 2000. 86 (3): p. 385-92.
108. Panelli, M. C., et al., Phase 1 study in patients with metastatic melanoma of immunization with dendritic cells presenting epitopes derived from the melanoma-associated antigens MART-1 and gp100. J Immunother, 2000. 23 (4): p. 487-98.
109. Schuler-Thurner, B., et al., Mage-3 and influenza-matrix peptide-specific cytotoxic T cells are inducible in terminal stage HLA-A2.1+ melanoma patients by mature monocyte-derived dendritic cells. J Immunol, 2000. 165 (6): p. 3492-6.
110. Lau, R., et al., Phase I trial of intravenous peptide-pulsed dendritic cells in patients with metastatic melanoma. J Immunother, 2001. 24 (1): p. 66-78.
111. Banchereau, J., et al., Immune and clinical responses in patients with metastatic melanoma to CD34 (+) progenitor-derived dendritic cell vaccine. Cancer Res, 2001. 61 (17): p. 6451-8.
112. Schuler-Thurner, B., et al., Rapid induction of tumor-specific type 1 T helper cells in metastatic melanoma patients by vaccination with mature, cryopreserved, peptide-loaded monocyte-derived dendritic cells. J Exp Med, 2002. 195 (10): p. 1279-88.
113. Palucka, A. K., et al., Single injection of CD34+ progenitor-derived dendritic cell vaccine can lead to induction of T-cell immunity in patients with stage IV melanoma. J Immunother, 2003. 26 (5): p. 432-9.
114. Bedrosian, I., et al., Intranodal administration of peptide-pulsed mature dendritic cell vaccines results in superior CD8+ T-cell function in melanoma patients. J Clin Oncol, 2003. 21 (20): p. 3826-35.
115. Slingluff, C. L., Jr., et al., Clinical and immunologic results of a randomized phase II trial of vaccination using four melanoma peptides either administered in granulocyte-macrophage colony-stimulating factor in adjuvant or pulsed on dendritic cells. J Clin Oncol, 2003. 21 (21): p. 4016-26.
116. Hersey, P., et al., Phase I/II study of treatment with dendritic cell vaccines in patients with disseminated melanoma. Cancer Immunol Immunother, 2004. 53 (2): p. 125-34.
117. Vilella, R., et al., Pilot study of treatment of biochemotherapy-refractory stage IV melanoma patients with autologous dendritic cells pulsed with a heterologous melanoma cell line lysate. Cancer Immunol Immunother, 2004. 53 (7): p. 651-8.
118. Palucka, A. K., et al., Spontaneous proliferation and type 2 cytokine secretion by CD4+ T cells in patients with metastatic melanoma vaccinated with antigen-pulsed dendritic cells. J Clin Immunol, 2005. 25 (3): p. 288-95.
119. Banchereau, J., et al., Immune and clinical outcomes in patients with stage IV melanoma vaccinated with peptide-pulsed dendritic cells derived from CD34+ progenitors and activated with type I interferon. J Immunother, 2005. 28 (5): p. 505-16.
120. Trakatelli, M., et al., A new dendritic cell vaccine generated with interleukin-3 and interferon-beta induces CD8+ T cell responses against NA17-A2 tumor peptide in melanoma patients. Cancer Immunol Immunother, 2006. 55 (4): p. 469-74.
121. Salcedo, M., et al., Vaccination of melanoma patients using dendritic cells loaded with an allogeneic tumor cell lysate. Cancer Immunol Immunother, 2006. 55 (7): p. 819-29.
122. Linette, G. P., et al., Immunization using autologous dendritic cells pulsed with the melanoma-associated antigen gp100-derived G280-9V peptide elicits CD8+ immunity. Clin Cancer Res, 2005. 11 (21): p. 7692-9.
123. Escobar, A., et al., Dendritic cell immunizations alone or combined with low doses of interleukin-2 induce specific immune responses in melanoma patients. Clin Exp Immunol, 2005. 142 (3): p. 555-68.
124. Tuettenberg, A., et al., Induction of strong and persistent MelanA/MART-1-specific immune responses by adjuvant dendritic cell-based vaccination of stage II melanoma patients. Int J Cancer, 2006. 118 (10): p. 2617-27.
125. Schadendorf, D., et al., Dacarbazine (DTIC) versus vaccination with autologous peptide-pulsed dendritic cells (DC) in first-line treatment of patients with metastatic melanoma: a randomized phase III trial of the DC study group of the DeCOG. Ann Oncol, 2006. 17 (4): p. 563-70.
126. Di Pucchio, T., et al., Immunization of stage IV melanoma patients with Melan-A/MART-1 and gp100 peptides plus IFN-alpha results in the activation of specific CD8 (+) T cells and monocyte/dendritic cell precursors. Cancer Res, 2006. 66 (9): p. 4943-51.
127. Nakai, N., et al., Vaccination of Japanese patients with advanced melanoma with peptide, tumor lysate or both peptide and tumor lysate-pulsed mature, monocyte-derived dendritic cells. J Dermatol, 2006. 33 (7): p. 462-72.
128. Palucka, A. K., et al., Dendritic cells loaded with killed allogeneic melanoma cells can induce objective clinical responses and MART-1 specific CD8+T-cell immunity. J Immunother, 2006. 29 (5): p. 545-57.
129. Lesimple, T., et al., Immunologic and clinical effects of injecting mature peptide-loaded dendritic cells by intralymphatic and intranodal routes in metastatic melanoma patients. Clin Cancer Res, 2006. 12 (24): p. 7380-8.
130. Guo, J., et al., Intratumoral injection of dendritic cells in combination with local hyperthermia induces systemic antitumor effect in patients with advanced melanoma. Int J Cancer, 2007. 120 (11): p. 2418-25.
131. O'Rourke, M. G., et al., Dendritic cell immunotherapy for stage IV melanoma. Melanoma Res, 2007. 17 (5): p. 316-22.
132. Bercovici, N., et al., Analysis and characterization of antitumor T-cell response after administration of dendritic cells loaded with allogeneic tumor lysate to metastatic melanoma patients. J Immunother, 2008. 31 (1): p. 101-12.
133. Hersey, P., et al., Phase I/II study of treatment with matured dendritic cells with or without low dose IL-2 in patients with disseminated melanoma. Cancer Immunol Immunother, 2008. 57 (7): p. 1039-51.
134. von Euw, E. M., et al., A phase I clinical study of vaccination of melanoma patients with dendritic cells loaded with allogeneic apoptotic/necrotic melanoma cells. Analysis of toxicity and immune response to the vaccine and of IL-10-1082 promoter genotype as predictor of disease progression. J Transl Med, 2008. 6: p. 6.
135. Carrasco, J., et al., Vaccination of a melanoma patient with mature dendritic cells pulsed with MAGE-3 peptides triggers the activity of nonvaccine anti-tumor cells. J Immunol, 2008. 180 (5): p. 3585-93.
136. Redman, B. G., et al., Phase Ib trial assessing autologous, tumor-pulsed dendritic cells as a vaccine administered with or without IL-2 in patients with metastatic melanoma. J Immunother, 2008. 31 (6): p. 591-8.
137. Daud, A. I., et al., Phenotypic and functional analysis of dendritic cells and clinical outcome in patients with high-risk melanoma treated with adjuvant granulocyte macrophage colony-stimulating factor. J Clin Oncol, 2008. 26 (19): p. 3235-41.
138. Engell-Noerregaard, L., et al., Review of clinical studies on dendritic cell-based vaccination of patients with malignant melanoma: assessment of correlation between clinical response and vaccine parameters. Cancer Immunol Immunother, 2009. 58 (1): p. 1-14.
139. Nakai, N., et al., Immunohistological analysis of peptide-induced delayed-type hypersensitivity in advanced melanoma patients treated with melanoma antigen-pulsed mature monocyte-derived dendritic cell vaccination. J Dermatol Sci, 2009. 53 (1): p. 40-7.
140. Dillman, R. O., et al., Phase II trial of dendritic cells loaded with antigens from self-renewing, proliferating autologous tumor cells as patient-specific antitumor vaccines in patients with metastatic melanoma: final report. Cancer Biother Radiopharm, 2009. 24 (3): p. 311-9.
141. Chang, J. W., et al., Immunotherapy with dendritic cells pulsed by autologous dactinomycin-induced melanoma apoptotic bodies for patients with malignant melanoma. Melanoma Res, 2009. 19 (5): p. 309-15.
142. Trepiakas, R., et al., Vaccination with autologous dendritic cells pulsed with multiple tumor antigens for treatment of patients with malignant melanoma: results from a phase I/II trial. Cytotherapy, 2010. 12 (6): p. 721-34.
143. Jacobs, J. F., et al., Dendritic cell vaccination in combination with anti-CD25 monoclonal antibody treatment: a phase I/II study in metastatic melanoma patients. Clin Cancer Res, 2010. 16 (20): p. 5067-78.
144. Ribas, A., et al., Multicenter phase II study of matured dendritic cells pulsed with melanoma cell line lysates in patients with advanced melanoma. J Transl Med, 2010. 8: p. 89.
145. Ridolfi, L., et al., Unexpected high response rate to traditional therapy after dendritic cell-based vaccine in advanced melanoma: update of clinical outcome and subgroup analysis. Clin Dev Immunol, 2010. 2010: p. 504979.
146. Cornforth, A. N., et al., Resistance to the proapoptotic effects of interferon-gamma on melanoma cells used in patient-specific dendritic cell immunotherapy is associated with improved overall survival. Cancer Immunol Immunother, 2011. 60 (1): p. 123-31.
147. Lesterhuis, W. J., et al., Wild-type and modified gp100 peptide-pulsed dendritic cell vaccination of advanced melanoma patients can lead to long-term clinical responses independent of the peptide used. Cancer Immunol Immunother, 2011. 60 (2): p. 249-60.
148. Bjoern, J., et al., Changes in peripheral blood level of regulatory T cells in patients with malignant melanoma during treatment with dendritic cell vaccination and low-dose IL-2. Scand J Immunol, 2011. 73 (3): p. 222-33.
149. Steele, J. C., et al., Phase I/II trial of a dendritic cell vaccine transfected with DNA encoding melan A and gp100 for patients with metastatic melanoma. Gene Ther, 2011. 18 (6): p. 584-93.
150. Kim, D. S., et al., Immunotherapy of malignant melanoma with tumor lysate-pulsed autologous monocyte-derived dendritic cells. Yonsei Med J, 2011. 52 (6): p. 990-8.
151. Ellebaek, E., et al., Metastatic melanoma patients treated with dendritic cell vaccination, Interleukin-2 and metronomic cyclophosphamide: results from a phase II trial. Cancer Immunol Immunother, 2012. 61 (10): p. 1791-804.
152. Dillman, R. O., et al., Tumor stem cell antigens as consolidative active specific immunotherapy: a randomized phase II trial of dendritic cells versus tumor cells in patients with metastatic melanoma. J Immunother, 2012. 35 (8): p. 641-9.
153. Dannull, J., et al., Melanoma immunotherapy using mature DCs expressing the constitutive proteasome. J Clin Invest, 2013. 123 (7): p. 3135-45.
154. Finkelstein, S. E., et al., Combination of external beam radiotherapy (EBRT) with intratumoral injection of dendritic cells as neo-adjuvant treatment of high-risk soft tissue sarcoma patients. Int J Radiat Oncol Biol Phys, 2012. 82 (2): p. 924-32.
155. Stift, A., et al., Dendritic cell vaccination in medullary thyroid carcinoma. Clin Cancer Res, 2004. 10 (9): p. 2944-53.
156. Kuwabara, K., et al., Results of a phase I clinical study using dendritic cell vaccinations for thyroid cancer. Thyroid, 2007. 17 (1): p. 53-8.
157. Bachleitner-Hofmann, T., et al., Pilot trial of autologous dendritic cells loaded with tumor lysate(s) from allogeneic tumor cell lines in patients with metastatic medullary thyroid carcinoma. Oncol Rep, 2009. 21 (6): p. 1585-92.
158. Yu, J. S., et al., Vaccination of malignant glioma patients with peptide-pulsed dendritic cells elicits systemic cytotoxicity and intracranial T-cell infiltration. Cancer Res, 2001. 61 (3): p. 842-7.
159. Yamanaka, R., et al., Vaccination of recurrent glioma patients with tumour lysate-pulsed dendritic cells elicits immune responses: results of a clinical phase I/II trial. Br J Cancer, 2003. 89 (7): p. 1172-9.
160. Yu, J. S., et al., Vaccination with tumor lysate-pulsed dendritic cells elicits antigen-specific, cytotoxic T-cells in patients with malignant glioma. Cancer Res, 2004. 64 (14): p. 4973-9.
161. Yamanaka, R., et al., Tumor lysate and IL-18 loaded dendritic cells elicits Th1 response, tumor-specific CD8+ cytotoxic T cells in patients with malignant glioma. J Neurooncol, 2005. 72 (2): p. 107-13.
162. Yamanaka, R., et al., Clinical evaluation of dendritic cell vaccination for patients with recurrent glioma: results of a clinical phase I/II trial. Clin Cancer Res, 2005. 11 (11): p. 4160-7.
163. Liau, L. M., et al., Dendritic cell vaccination in glioblastoma patients induces systemic and intracranial T-cell responses modulated by the local central nervous system tumor microenvironment. Clin Cancer Res, 2005. 11 (15): p. 5515-25.
164. Walker, D. G., et al., Results of a phase I dendritic cell vaccine trial for malignant astrocytoma: potential interaction with adjuvant chemotherapy. J Clin Neurosci, 2008. 15 (2): p. 114-21.
165. Leplina, O. Y., et al., Use of interferon-alpha-induced dendritic cells in the therapy of patients with malignant brain gliomas. Bull Exp Biol Med, 2007. 143 (4): p. 528-34.
166. De Vleeschouwer, S., et al., Postoperative adjuvant dendritic cell-based immunotherapy in patients with relapsed glioblastoma multiforme. Clin Cancer Res, 2008. 14 (10): p. 3098-104.
167. Ardon, H., et al., Adjuvant dendritic cell-based tumour vaccination for children with malignant brain tumours. Pediatr Blood Cancer, 2010. 54 (4): p. 519-25.
168. Prins, R. M., et al., Gene expression profile correlates with T-cell infiltration and relative survival in glioblastoma patients vaccinated with dendritic cell immunotherapy. Clin Cancer Res, 2011. 17 (6): p. 1603-15.
169. Okada, H., et al., Induction of CD8+ T-cell responses against novel glioma-associated antigen peptides and clinical activity by vaccinations with {alpha}-type 1 polarized dendritic cells and polyinosinic-polycytidylic acid stabilized by lysine and carboxymethylcellulose in patients with recurrent malignant glioma. J Clin Oncol, 2011. 29 (3): p. 330-6.
170. Fadul, C. E., et al., Immune response in patients with newly diagnosed glioblastoma multiforme treated with intranodal autologous tumor lysate-dendritic cell vaccination after radiation chemotherapy. J Immunother, 2011. 34 (4): p. 382-9.
171. Chang, C. N., et al., A phase I/II clinical trial investigating the adverse and therapeutic effects of a postoperative autologous dendritic cell tumor vaccine in patients with malignant glioma. J Clin Neurosci, 2011. 18 (8): p. 1048-54.
172. Cho, D. Y., et al., Adjuvant immunotherapy with whole-cell lysate dendritic cells vaccine for glioblastoma multiforme: a phase II clinical trial. World Neurosurg, 2012. 77 (5-6): p. 736-44.
173. Iwami, K., et al., Peptide-pulsed dendritic cell vaccination targeting interleukin-13 receptor alpha2 chain in recurrent malignant glioma patients with HLA-A*24/A*02 allele. Cytotherapy, 2012. 14 (6): p. 733-42.
174. Fong, B., et al., Monitoring of regulatory T cell frequencies and expression of CTLA-4 on T cells, before and after DC vaccination, can predict survival in GBM patients. PLOS One, 2012. 7 (4): p. e32614.
175. De Vleeschouwer, S., et al., Stratification according to HGG-IMMUNO RPA model predicts outcome in a large group of patients with relapsed malignant glioma treated by adjuvant postoperative dendritic cell vaccination. Cancer Immunol Immunother, 2012. 61 (11): p. 2105-12.
176. Phuphanich, S., et al., Phase I trial of a multi-epitope-pulsed dendritic cell vaccine for patients with newly diagnosed glioblastoma. Cancer Immunol Immunother, 2013. 62 (1): p. 125-35.
177. Akiyama, Y., et al., alpha-type-1 polarized dendritic cell-based vaccination in recurrent high-grade glioma: a phase I clinical trial. BMC Cancer, 2012. 12: p. 623.
178. Prins, R. M., et al., Comparison of glioma-associated antigen peptide-loaded versus autologous tumor lysate-loaded dendritic cell vaccination in malignant glioma patients. J Immunother, 2013. 36 (2): p. 152-7.
179. Shah, A. H., et al., Dendritic cell vaccine for recurrent high-grade gliomas in pediatric and adult subjects: clinical trial protocol. Neurosurgery, 2013. 73 (5): p. 863-7.
180. Reichardt, V. L., et al., Idiotype vaccination using dendritic cells after autologous peripheral blood stem cell transplantation for multiple myeloma—a feasibility study. Blood, 1999. 93 (7): p. 2411-9.
181. Lim, S. H. and R. Bailey-Wood, Idiotypic protein-pulsed dendritic cell vaccination in multiple myeloma. Int J Cancer, 1999. 83 (2): p. 215-22.
182. Motta, M. R., et al., Generation of dendritic cells from CD14+ monocytes positively selected by immunomagnetic adsorption for multiple myeloma patients enrolled in a clinical trial of anti-idiotype vaccination. Br J Haematol, 2003. 121 (2): p. 240-50.
183. Reichardt, V. L., et al., Idiotype vaccination of multiple myeloma patients using monocyte-derived dendritic cells. Haematologica, 2003. 88 (10): p. 1139-49.
184. Guardino, A. E., et al., Production of myeloid dendritic cells (DC) pulsed with tumor-specific idiotype protein for vaccination of patients with multiple myeloma. Cytotherapy, 2006. 8 (3): p. 277-89.
185. Lacy, M. Q., et al., Idiotype-pulsed antigen-presenting cells following autologous transplantation for multiple myeloma may be associated with prolonged survival. Am J Hematol, 2009. 84 (12): p. 799-802.
186. Yi, Q., et al., Optimizing dendritic cell-based immunotherapy in multiple myeloma: intranodal injections of idiotype-pulsed CD40 ligand-matured vaccines led to induction of type-1 and cytotoxic T-cell immune responses in patients. Br J Haematol, 2010. 150 (5): p. 554-64.
187. Rollig, C., et al., Induction of cellular immune responses in patients with stage-I multiple myeloma after vaccination with autologous idiotype-pulsed dendritic cells. J Immunother, 2011. 34 (1): p. 100-6.
188. Zahradova, L., et al., Efficacy and safety of Id-protein-loaded dendritic cell vaccine in patients with multiple myeloma—phase II study results. Neoplasma, 2012. 59 (4): p. 440-9.
189. Timmerman, J. M., et al., Idiotype-pulsed dendritic cell vaccination for B-cell lymphoma: clinical and immune responses in 35 patients. Blood, 2002. 99 (5): p. 1517-26.
190. Maier, T., et al., Vaccination of patients with cutaneous T-cell lymphoma using intranodal injection of autologous tumor-lysate-pulsed dendritic cells. Blood, 2003. 102 (7): p. 2338-44.
191. Di Nicola, M., et al., Vaccination with autologous tumor-loaded dendritic cells induces clinical and immunologic responses in indolent B-cell lymphoma patients with relapsed and measurable disease: a pilot study. Blood, 2009. 113 (1): p. 18-27.
192. Hus, I., et al., Allogeneic dendritic cells pulsed with tumor lysates or apoptotic bodies as immunotherapy for patients with early-stage B-cell chronic lymphocytic leukemia. Leukemia, 2005. 19 (9): p. 1621-7.
193. Li, L., et al., Immunotherapy for patients with acute myeloid leukemia using autologous dendritic cells generated from leukemic blasts. Int J Oncol, 2006. 28 (4): p. 855-61.
194. Roddie, H., et al., Phase I/II study of vaccination with dendritic-like leukaemia cells for the immunotherapy of acute myeloid leukaemia. Br J Haematol, 2006. 133 (2): p. 152-7.
195. Litzow, M. R., et al., Testing the safety of clinical-grade mature autologous myeloid DC in a phase I clinical immunotherapy trial of CML. Cytotherapy, 2006. 8 (3): p. 290-8.
196. Westermann, J., et al., Vaccination with autologous non-irradiated dendritic cells in patients with bcr/abl+ chronic myeloid leukaemia. Br J Haematol, 2007. 137 (4): p. 297-306.
197. Hus, I., et al., Vaccination of B-CLL patients with autologous dendritic cells can change the frequency of leukemia antigen-specific CD8+ T cells as well as CD4+CD25+FoxP3+ regulatory T cells toward an antileukemia response. Leukemia, 2008. 22 (5): p. 1007-17.
198. Palma, M., et al., Development of a dendritic cell-based vaccine for chronic lymphocytic leukemia. Cancer Immunol Immunother, 2008. 57 (11): p. 1705-10.
199. Van Tendeloo, V. F., et al., Induction of complete and molecular remissions in acute myeloid leukemia by Wilms' tumor 1 antigen-targeted dendritic cell vaccination. Proc Natl Acad Sci USA, 2010. 107 (31): p. 13824-9.
200. Iwashita, Y., et al., A phase I study of autologous dendritic cell-based immunotherapy for patients with unresectable primary liver cancer. Cancer Immunol Immunother, 2003. 52 (3): p. 155-61.
201. Lee, W. C., et al., Vaccination of advanced hepatocellular carcinoma patients with tumor lysate-pulsed dendritic cells: a clinical trial. J Immunother, 2005. 28 (5): p. 496-504.
202. Butterfield, L. H., et al., A phase I/II trial testing immunization of hepatocellular carcinoma patients with dendritic cells pulsed with four alpha-fetoprotein peptides. Clin Cancer Res, 2006. 12 (9): p. 2817-25.
203. Palmer, D. H., et al., A phase II study of adoptive immunotherapy using dendritic cells pulsed with tumor lysate in patients with hepatocellular carcinoma. Hepatology, 2009. 49 (1): p. 124-32.
204. El Ansary, M., et al., Immunotherapy by autologous dendritic cell vaccine in patients with advanced HCC. J Cancer Res Clin Oncol, 2013. 139 (1): p. 39-48.
205. Tada, F., et al., Phase I/II study of immunotherapy using tumor antigen-pulsed dendritic cells in patients with hepatocellular carcinoma. Int J Oncol, 2012. 41 (5): p. 1601-9.
206. Ueda, Y., et al., Dendritic cell-based immunotherapy of cancer with carcinoembryonic antigen-derived, HLA-A24-restricted CTL epitope: Clinical outcomes of 18 patients with metastatic gastrointestinal or lung adenocarcinomas. Int J Oncol, 2004. 24 (4): p. 909-17.
207. Hirschowitz, E. A., et al., Autologous dendritic cell vaccines for non-small-cell lung cancer. J Clin Oncol, 2004. 22 (14): p. 2808-15.
208. Chang, G. C., et al., A pilot clinical trial of vaccination with dendritic cells pulsed with autologous tumor cells derived from malignant pleural effusion in patients with late-stage lung carcinoma. Cancer, 2005. 103 (4): p. 763-71.
209. Yannelli, J. R., et al., The large scale generation of dendritic cells for the immunization of patients with non-small cell lung cancer (NSCLC). Lung Cancer, 2005. 47 (3): p. 337-50.
210. Ishikawa, A., et al., A phase I study of alpha-galactosylceramide (KRN7000)-pulsed dendritic cells in patients with advanced and recurrent non-small cell lung cancer. Clin Cancer Res, 2005. 11 (5): p. 1910-7.
211. Antonia, S. J., et al., Combination of p53 cancer vaccine with chemotherapy in patients with extensive stage small cell lung cancer. Clin Cancer Res, 2006. 12 (3 Pt 1): p. 878-87.
212. Perrot, I., et al., Dendritic cells infiltrating human non-small cell lung cancer are blocked at immature stage. J Immunol, 2007. 178 (5): p. 2763-9.
213. Hirschowitz, E. A., et al., Immunization of NSCLC patients with antigen-pulsed immature autologous dendritic cells. Lung Cancer, 2007. 57 (3): p. 365-72.
214. Baratelli, F., et al., Pre-clinical characterization of GMP grade CCL21-gene modified dendritic cells for application in a phase I trial in non-small cell lung cancer. J Transl Med, 2008. 6: p. 38.
215. Hegmans, J. P., et al., Consolidative dendritic cell-based immunotherapy elicits cytotoxicity against malignant mesothelioma. Am J Respir Crit Care Med, 2010. 181 (12): p. 1383-90.
216. Um, S. J., et al., Phase I study of autologous dendritic cell tumor vaccine in patients with non-small cell lung cancer. Lung Cancer, 2010. 70 (2): p. 188-94.
217. Chiappori, A. A., et al., INGN-225: a dendritic cell-based p53 vaccine (Ad.p53-DC) in small cell lung cancer: observed association between immune response and enhanced chemotherapy effect. Expert Opin Biol Ther, 2010. 10 (6): p. 983-91.
218. Perroud, M. W., Jr., et al., Mature autologous dendritic cell vaccines in advanced non-small cell lung cancer: a phase I pilot study. J Exp Clin Cancer Res, 2011. 30: p. 65.
219. Skachkova, O. V., et al., Immunological markers of anti-tumor dendritic cells vaccine efficiency in patients with non-small cell lung cancer. Exp Oncol, 2013. 35 (2): p. 109-13.
220. Hernando, J. J., et al., Vaccination with autologous tumour antigen-pulsed dendritic cells in advanced gynaecological malignancies: clinical and immunological evaluation of a phase I trial. Cancer Immunol Immunother, 2002. 51 (1): p. 45-52.
221. Rahma, O. E., et al., A gynecologic oncology group phase II trial of two p53 peptide vaccine approaches: subcutaneous injection and intravenous pulsed dendritic cells in high recurrence risk ovarian cancer patients. Cancer Immunol Immunother, 2012. 61 (3): p. 373-84.
222. Chu, C. S., et al., Phase I/II randomized trial of dendritic cell vaccination with or without cyclophosphamide for consolidation therapy of advanced ovarian cancer in first or second remission. Cancer Immunol Immunother, 2012. 61 (5): p. 629-41.
223. Kandalaft, L. E., et al., A Phase I vaccine trial using dendritic cells pulsed with autologous oxidized lysate for recurrent ovarian cancer. J Transl Med, 2013. 11: p. 149.
224. Lepisto, A. J., et al., A phase I/II study of a MUC1 peptide pulsed autologous dendritic cell vaccine as adjuvant therapy in patients with resected pancreatic and biliary tumors. Cancer Ther, 2008. 6 (B): p. 955-964.
225. Rong, Y., et al., A phase I pilot trial of MUC1-peptide-pulsed dendritic cells in the treatment of advanced pancreatic cancer. Clin Exp Med, 2012. 12 (3): p. 173-80.
226. Endo, H., et al., Phase I trial of preoperative intratumoral injection of immature dendritic cells and OK-432 for resectable pancreatic cancer patients. J Hepatobiliary Pancreat Sci, 2012. 19 (4): p. 465-75.
227. Zumwalt, T. J., et al., Active secretion of CXCL10 and CCL5 from colorectal cancer microenvironments associates with GranzymeB+CD8+ T-cell infiltration. Oncotarget, 2015. 6 (5): p. 2981-91.
228. Ochsenbein, A. F., Principles of tumor immunosurveillance and implications for immunotherapy. Cancer Gene Ther, 2002. 9 (12): p. 1043-55.
229. Ochsenbein, A. F., et al., Roles of tumour localization, second signals and cross priming in cytotoxic T-cell induction. Nature, 2001. 411 (6841): p. 1058-64.
230. Buhtoiarov, I. N., et al., CD40 ligation activates murine macrophages via an IFN-gamma-dependent mechanism resulting in tumor cell destruction in vitro. J Immunol, 2005. 174 (10): p. 6013-22.
231. Egilmez, N. K., et al., Human CD4+ effector T cells mediate indirect interleukin-12- and interferon-gamma-dependent suppression of autologous HLA-negative lung tumor xenografts in severe combined immunodeficient mice. Cancer Res, 2002. 62 (9): p. 2611-7.
232. Pace, J. L., et al., Recombinant mouse gamma interferon induces the priming step in macrophage activation for tumor cell killing. J Immunol, 1983. 130 (5): p. 2011-3.
233. Heusinkveld, M., et al., M2 macrophages induced by prostaglandin E2 and IL-6 from cervical carcinoma are switched to activated M1 macrophages by CD4+Th1 cells. J Immunol, 2011. 187 (3): p. 1157-65.
234. Hu, G. and S. Wang, Tumor-infiltrating CD45RO+ Memory T Lymphocytes Predict Favorable Clinical Outcome in Solid Tumors. Sci Rep, 2017. 7 (1): p. 10376.
235. Lohneis, P., et al., Cytotoxic tumour-infiltrating T lymphocytes influence outcome in resected pancreatic ductal adenocarcinoma. Eur J Cancer, 2017. 83: p. 290-301.
236. Liu, S., et al., Role of Cytotoxic Tumor-Infiltrating Lymphocytes in Predicting Outcomes in Metastatic HER2-Positive Breast Cancer: A Secondary Analysis of a Randomized Clinical Trial. JAMA Oncol, 2017: p. e172085.
237. Berntsson, J., et al., The clinical impact of tumour-infiltrating lymphocytes in colorectal cancer differs by anatomical subsite: A cohort study. Int J Cancer, 2017. 141 (8): p. 1654-1666.
238. Xu, Y., et al., Higher Numbers of T-Bet+ Tumor-Infiltrating Lymphocytes Associate with Better Survival in Human Epithelial Ovarian Cancer. Cell Physiol Biochem, 2017. 41 (2): p. 475-483.
239. Melief, S. M., et al., Long-term Survival and Clinical Benefit from Adoptive T-cell Transfer in Stage IV Melanoma Patients Is Determined by a Four-Parameter Tumor Immune Signature. Cancer Immunol Res, 2017. 5 (2): p. 170-179.
240. Scurr, M. J., et al., Low-dose cyclophosphamide induces anti-tumor T-cell responses which associate with survival in metastatic colorectal cancer. Clin Cancer Res, 2017.
241. Wang, L., et al., Arsenic trioxide is an immune adjuvant in liver cancer treatment. Mol Immunol, 2017. 81: p. 118-126.
242. Ouyang, Z., et al., Regulatory T cells in the immunotherapy of melanoma. Tumour Biol, 2016. 37 (1): p. 77-85.
243. Dimeloe, S., et al., Human regulatory T cells lack the cyclophosphamide-extruding transporter ABCB1 and are more susceptible to cyclophosphamide-induced apoptosis. Eur J Immunol, 2014. 44 (12): p. 3614-20.
244. Camisaschi, C., et al., Effects of cyclophosphamide and IL-2 on regulatory CD4+ T cell frequency and function in melanoma patients vaccinated with HLA-class I peptides: impact on the antigen-specific T cell response. Cancer Immunol Immunother, 2013. 62 (5): p. 897-908.
245. Kan, S., et al., Suppressive effects of cyclophosphamide and gemcitabine on regulatory T-cell induction in vitro. Anticancer Res, 2012. 32 (12): p. 5363-9.
246. Farsam, V., et al., Antitumor and immunomodulatory properties of artemether and its ability to reduce CD4+CD25+FoxP3+ T reg cells in vivo. Int Immunopharmacol, 2011. 11 (11): p. 1802-8.
247. Lawrence, H. S. and A. M. Pappenheimer, Jr., Transfer of delayed hypersensitivity to diphtheria toxin in man. J Exp Med, 1956. 104 (3): p. 321-35.
248. Rosenfeld, S. and D. Dressler, Transfer factor: a subcellular component that transmits information for specific immune responses. Proc Natl Acad Sci USA, 1974. 71 (6): p. 2473-7.
249. Dressler, D. and S. Rosenfeld, On the chemical nature of transfer factor. Proc Natl Acad Sci USA, 1974. 71 (11): p. 4429-34.
250. Shifrine, M. and R. Scibienski, Transfer factor-hypotheses for its structure and function. Oncology, 1975. 32 (5-6): p. 269-74.
251. Kirkpatrick, C. H., Properties and activities of transfer factor. J Allergy Clin Immunol, 1975. 55 (6): p. 411-21.
252. Burger, D. R., et al., Human transfer factor: fractionation and biologic activity. J Immunol, 1976. 117 (3): p. 789-96.
253. Berron-Perez, R., et al., Indications, usage, and dosage of the transfer factor. Rev Alerg Mex, 2007. 54 (4): p. 134-9.
254. Alexandrescu, D. T., C. A. Dasanu, and C. L. Kauffman, Acute scurvy during treatment with interleukin-2. Clin Exp Dermatol, 2009. 34 (7): p. 811-4.
255. Anthony, H. M. and C. J. Schorah, Severe hypovitaminosis C in lung-cancer patients: the utilization of vitamin C in surgical repair and lymphocyte-related host resistance. Br J Cancer, 1982. 46 (3): p. 354-67.
256. McMurray, D. N., Cell-mediated immunity in nutritional deficiency. Prog Food Nutr Sci, 1984. 8 (3-4): p. 193-228.
257. highbeam.com/doc/1G1-186526887.html.
258. Marcus, S. L., et al., Severe hypovitaminosis C occurring as the result of adoptive immunotherapy with high-dose interleukin 2 and lymphokine-activated killer cells. Cancer Res, 1987. 47 (15): p. 4208-12.
259. Marcus, S. L., et al., Hypovitaminosis C in patients treated with high-dose interleukin 2 and lymphokine-activated killer cells. Am J Clin Nutr, 1991. 54 (6 Suppl): p. 1292S-1297S.
260. Yeom, C. H., G. C. Jung, and K. J. Song, Changes of terminal cancer patients' health-related quality of life after high dose vitamin C administration. J Korean Med Sci, 2007. 22 (1): p. 7-11.
261. Murata, A., F. Morishige, and H. Yamaguchi, Prolongation of survival times of terminal cancer patients by administration of large doses of ascorbate. Int J Vitam Nutr Res Suppl, 1982. 23: p. 103-13.
262. Cameron, E. and A. Campbell, The orthomolecular treatment of cancer. II. Clinical trial of high-dose ascorbic acid supplements in advanced human cancer. Chem Biol Interact, 1974. 9 (4): p. 285-315.
263. Riordan, N. H., et al., Intravenous ascorbate as a tumor cytotoxic chemotherapeutic agent. Med Hypotheses, 1995. 44 (3): p. 207-13.
264. Deubzer, B., et al., H(2)O(2)-mediated cytotoxicity of pharmacologic ascorbate concentrations to neuroblastoma cells: potential role of lactate and ferritin. Cell Physiol Biochem. 25 (6): p. 767-74.
265. Gilloteaux, J., et al., Cell damage and death by autoschizis in human bladder (RT4) carcinoma cells resulting from treatment with ascorbate and menadione. Ultrastruct Pathol. 34 (3): p. 140-60.
266. Cullen, J. J., Ascorbate induces autophagy in pancreatic cancer. Autophagy. 6 (3): p. 421-2.
267. Takemura, Y., et al., High dose of ascorbic acid induces cell death in mesothelioma cells. Biochem Biophys Res Commun. 394 (2): p. 249-53.
268. Verrax, J., et al., In situ modulation of oxidative stress: a novel and efficient strategy to kill cancer cells. Curr Med Chem, 2009. 16 (15): p. 1821-30.
269. Fromberg, A., et al., Ascorbate exerts anti-proliferative effects through cell cycle inhibition and sensitizes tumor cells towards cytostatic drugs. Cancer Chemother Pharmacol. 270. Pollard, H. B., et al., Pharmacological ascorbic acid suppresses syngeneic tumor growth and metastases in hormone-refractory prostate cancer. In Vivo. 24 (3): p. 249-55.
271. Chen, Q., et al., Pharmacologic doses of ascorbate act as a prooxidant and decrease growth of aggressive tumor xenografts in mice. Proc Natl Acad Sci USA, 2008. 105 (32): p. 11105-9.
272. Padayatty, S. J., et al., Intravenously administered vitamin C as cancer therapy: three cases. CMAJ, 2006. 174 (7): p. 937-42.
273. Mikirova, N. A., T. E. Ichim, and N. H. Riordan, Anti-angiogenic effect of high doses of ascorbic acid. J Transl Med, 2008. 6: p. 50.
274. Ashino, H., et al., Novel function of ascorbic acid as an angiostatic factor. Angiogenesis, 2003. 6 (4): p. 259-69.
275. Mikirova, N. A., J. J. Casciari, and N. H. Riordan, Ascorbate inhibition of angiogenesis in aortic rings ex vivo and subcutaneous Matrigel plugs in vivo. J Angiogenes Res. 2: p. 2.
276. Yeom, C. H., et al., High dose concentration administration of ascorbic acid inhibits tumor growth in BALB/C mice implanted with sarcoma 180 cancer cells via the restriction of angiogenesis. J Transl Med, 2009. 7: p. 70.
277. Muellner, M. K., et al., Vitamin C inhibits NO-induced stabilization of HIF-1alpha in HUVECs. Free Radic Res. 44 (7): p. 783-91.
278. Horak, P., et al., Negative feedback control of HIF-1 through REDDI-regulated ROS suppresses tumorigenesis. Proc Natl Acad Sci USA. 107 (10): p.
4675-80.
279. Gao, P., et al., HIF-dependent antitumorigenic effect of antioxidants in vivo. Cancer Cell, 2007. 12 (3): p. 230-8.
280. Kuiper, C., et al., Low ascorbate levels are associated with increased hypoxia-inducible factor-1 activity and an aggressive tumor phenotype in endometrial cancer. Cancer Res. 70 (14): p. 5749-58.
281. clinicaltrials.gov/ct2/show/NCT00441207.
282. clinicaltrials.gov/ct2/show/NCT01080352.
283. clinicaltrials.gov/ct2/show/NCT00626444.
284. clinicaltrials.gov/ct2/show/NCT01125449.
285. clinicaltrials.gov/ct2/show/NCT01050621.
286. clinicaltrials.gov/ct2/show/NCT00954525.
287. Tatla, S., et al., The role of reactive oxygen species in triggering proliferation and IL-2 secretion in T cells. Free Radic Biol Med, 1999. 26 (1-2): p. 14-24.
288. Williams, M. S. and J. Kwon, T cell receptor stimulation, reactive oxygen species, and cell signaling. Free Radic Biol Med, 2004. 37 (8): p. 1144-51.
289. Schwager, J. and J. Schulze, Influence of ascorbic acid on the response to mitogens and interleukin production of porcine lymphocytes. Int J Vitam Nutr Res, 1997. 67 (1): p. 10-6.
290. Eylar, E., et al., Sustained levels of ascorbic acid are toxic and immunosuppressive for human T cells. P R Health Sci J, 1996. 15 (1): p. 21-6.
291. Huwyler, T., A. Hirt, and A. Morell, Effect of ascorbic acid on human natural killer cells. Immunol Lett, 1985. 10 (3-4): p. 173-6.
292. Tan, P. H., et al., Inhibition of NF-kappa B and oxidative pathways in human dendritic cells by antioxidative vitamins generates regulatory T cells. J Immunol, 2005. 174 (12): p. 7633-44.
293. Chen, S., L. Yang, and Y. Li, TCR zeta chain expression in T cells from patients with CML. Hematology, 2009. 14 (2): p. 95-100.
294. Kulkarni, D. P., et al., Mechanisms involved in the down-regulation of TCR zeta chain in tumor versus peripheral blood of oral cancer patients. Int J Cancer, 2009. 124 (7): p. 1605-13.
295. Gruber, I. V., et al., Down-regulation of CD28, TCR-zeta (zeta) and up-regulation of FAS in peripheral cytotoxic T-cells of primary breast cancer patients. Anticancer Res, 2008. 28 (2A): p. 779-84.
296. Pignataro, L., et al., Down-regulation of zeta chain and zeta-associated protein 70 (Zap 70) expression in circulating T lymphocytes in laryngeal squamous cell carcinoma. Anal Quant Cytol Histol, 2007. 29 (1): p. 57-62.
297. Zehbe, I., et al., Different T-cell receptor (TCR) zeta chain expression in cervical cancer and its precursor lesions. Zentralbl Gynakol, 2006. 128 (5): p. 266-70.
298. Ciszak, L., et al., Alterations in the expression of signal-transducing CD3 zeta chain in T cells from patients with chronic inflammatory/autoimmune diseases. Arch Immunol Ther Exp (Warsz), 2007. 55 (6): p. 373-86.
299. Baniyash, M., TCR zeta-chain downregulation: curtailing an excessive inflammatory immune response. Nat Rev Immunol, 2004. 4 (9): p. 675-87.
300. Pitcher, L. A. and N. S. van Oers, T-cell receptor signal transmission: who gives an ITAM? Trends Immunol, 2003. 24 (10): p. 554-60.
301. Gastman, B. R., et al., Tumor-induced apoptosis of T lymphocytes: elucidation of intracellular apoptotic events. Blood, 2000. 95 (6): p. 2015-23.
302. Boussiotis, V. A., et al., Differential association of protein tyrosine kinases with the T cell receptor is linked to the induction of anergy and its prevention by B7 family-mediated costimulation. J Exp Med, 1996. 184 (2): p. 365-76.
303. Kim, C. W., et al., Alteration of signal-transducing molecules and phenotypical characteristics in peripheral blood lymphocytes from gastric carcinoma patients. Pathobiology, 1999. 67 (3): p. 123-8.
304. Reichert, T. E., et al., Absent or low expression of the zeta chain in T cells at the tumor site correlates with poor survival in patients with oral carcinoma. Cancer Res, 1998. 58 (23): p. 5344-7.
305. Zea, A. H., et al., Alterations in T cell receptor and signal transduction molecules in melanoma patients. Clin Cancer Res, 1995. 1 (11): p. 1327-35.
306. Healy, C. G., et al., Impaired expression and function of signal-transducing zeta chains in peripheral T cells and natural killer cells in patients with prostate cancer. Cytometry, 1998. 32 (2): p. 109-19.
307. Mulder, W. M., et al., T cell receptor-zeta and granzyme B expression in mononuclear cell infiltrates in normal colon mucosa and colon carcinoma. Gut, 1997. 40 (1): p. 113-9.
308. Muller, D., et al., [The expression of zeta-chain of the T cell receptor as prognostic marker for patients with head and neck cancer]. Laryngorhinootologie, 2002. 81 (7): p. 516-20.
309. Whiteside, T. L., Down-regulation of zeta-chain expression in T cells: a biomarker of prognosis in cancer? Cancer Immunol Immunother, 2004. 53 (10): p. 865-78.
310. Eleftheriadis, T., et al., Decreased CD3+CD16+ natural killer-like T-cell percentage and zeta-chain expression accompany chronic inflammation in haemodialysis patients. Nephrology (Carlton), 2009. 14 (5): p. 471-5.
311. Eleftheriadis, T., et al., Chronic inflammation and CD16+ natural killer cell zeta-chain downregulation in hemodialysis patients. Blood Purif, 2008. 26 (4): p. 317-21.
312. Nambiar, M. P., et al., TCR zeta-chain abnormalities in human systemic lupus erythematosus. Methods Mol Med, 2004. 102: p. 49-72.
313. Takeuchi, T., et al., T cell abnormalities in systemic lupus erythematosus. Autoimmunity, 2005. 38 (5): p. 339-46.
314. Berg, L., et al., Down-regulation of the T cell receptor CD3 zeta chain in rheumatoid arthritis (RA) and its influence on T cell responsiveness. Clin Exp Immunol, 2000. 120 (1): p. 174-82.
315. Maurice, M. M., et al., Defective TCR-mediated signaling in synovial T cells in rheumatoid arthritis. J Immunol, 1997. 159 (6): p. 2973-8.
316. Ammirati, E., et al., Expansion of T-cell receptor zeta dim effector T cells in acute coronary syndromes. Arterioscler Thromb Vasc Biol, 2008. 28 (12): p. 2305-11.
317. Sikora, J., et al., The role of monocytes/macrophages in TCR-zeta chain downregulation and apoptosis of T lymphocytes in malignant pleural effusions. J Biol Regul Homeost Agents, 2004. 18 (1): p. 26-32.
318. Markiewski, M. M., et al., Modulation of the antitumor immune response by complement. Nat Immunol, 2008. 9 (11): p. 1225-35.
319. Corzo, C. A., et al., Mechanism regulating reactive oxygen species in tumor-induced myeloid-derived suppressor cells. J Immunol, 2009. 182 (9): p. 5693-701.
320. Choi, J. Y., J. A. Oughton, and N. I. Kerkvliet, Functional alterations in CD11b (+) Gr-1 (+) cells in mice injected with allogeneic tumor cells and treated with 2,3,7,8-tetrachlorodibenzo-p-dioxin. Int Immunopharmacol, 2003. 3 (4): p. 553-70.
321. Makarenkova, V. P., et al., CD11b+/Gr-1+ myeloid suppressor cells cause T cell dysfunction after traumatic stress. J Immunol, 2006. 176 (4): p. 2085-94.
322. Ezernitchi, A. V., et al., TCR zeta down-regulation under chronic inflammation is mediated by myeloid suppressor cells differentially distributed between various lymphatic organs. J Immunol, 2006. 177 (7): p. 4763-72.
323. Schmielau, J. and O. J. Finn, Activated granulocytes and granulocyte-derived hydrogen peroxide are the underlying mechanism of suppression of t-cell function in advanced cancer patients. Cancer Res, 2001. 61 (12): p. 4756-60.
324. Nambiar, M. P., et al., Oxidative stress is involved in the heat stress-induced downregulation of TCR zeta chain expression and TCR/CD3-mediated [Ca(2+)](i) response in human T-lymphocytes. Cell Immunol, 2002. 215 (2): p. 151-61.

Each of the references above-listed or otherwise mentioned in this specification is hereby incorporated by reference in its entirety and for all purposes.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

### Example 1: Exemplary Combination Immunotherapy Protocol #1

Female C57/BL6 mice were inoculated with 500,000 B16 melanoma cells intradermally. Cells were purchased from ATCC and grown in complete DMEM media.

After 5 days of tumor growth mice were administered: a) Poly IC (antigen nonspecific immune stimulant 100 ng/mouse, days 5, 6 and 7 after tumor inoculation (labeled “Control”); b) Poly IC plus B16 melanoma lysate (sonicated lysate of 1 million cells per mouse, at 5 days after tumor inoculation) (Group B); c) low dose cyclophosphamide (50 mg/kg) on days 5, 6 and 7 after tumor inoculation) (Group C) and d) Poly IC plus B16 melanoma lysate (sonicated lysate of 1 million cells per mouse, at 5 days after tumor inoculation plus cyclophosphamide) (Group D).

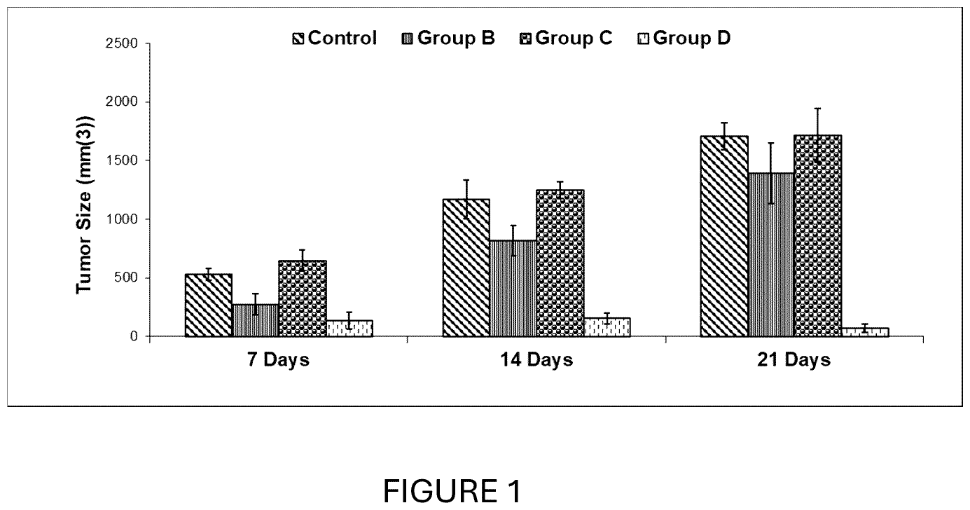

The below Table 1, Table 2 and Table 3 present results at day 14, 21, and 28 respectively, which are also illustrated in FIG. 1.

TABLE 1
day 14

| | Group A | Group B | Group C | Group D |
|---|---|---|---|---|
| 1.0 | 545.0 | 435.0 | 746.0 | 103.0 |
| 2.0 | 546.0 | 332.0 | 773.0 | 112.0 |
| 3.0 | 547.0 | 364.0 | 674.0 | 124.0 |
| 4.0 | 544.0 | 143.0 | 657.0 | 101.0 |
| 5.0 | 586.0 | 214.0 | 546.0 | 32.0 |
| 6.0 | 456.0 | 215.0 | 764.0 | 144.0 |
| 7.0 | 436.0 | 223.0 | 645.0 | 122.0 |
| 8.0 | 525.0 | 254.0 | 557.0 | 142.0 |
| 9.0 | 578.0 | 332.0 | 574.0 | 146.0 |
| 10.0 | 532.0 | 231.0 | 547.0 | 321.0 |
| Average | 529.5 | 274.3 | 648.3 | 134.7 |
| STDEV | 48.0 | 88.1 | 90.6 | 73.4 |

TABLE 2
day 21

| | Group A | Group B | Group C | Group D |
|---|---|---|---|---|
| 1.0 | 956.0 | 954.0 | 1123.0 | 113.0 |
| 2.0 | 1002.0 | 885.0 | 1214.0 | 221.0 |
| 3.0 | 1213.0 | 765.0 | 1325.0 | 164.0 |
| 4.0 | 1002.0 | 876.0 | 1121.0 | 137.0 |
| 5.0 | 1246.0 | 785.0 | 1363.0 | 135.0 |
| 6.0 | 1124.0 | 784.0 | 1214.0 | 121.0 |
| 7.0 | 1141.0 | 734.0 | 1242.0 | 154.0 |
| 8.0 | 1251.0 | 756.0 | 1215.0 | 124.0 |
| 9.0 | 1215.0 | 886.0 | 1125.0 | 253.0 |
| 10.0 | 1521.0 | 756.0 | 1543.0 | 125.0 |
| Average | 1167.1 | 818.1 | 1248.5 | 154.7 |
| STDEV | 164.8 | 75.1 | 131.9 | 46.6 |

TABLE 3
Day 28

| | Group A | Group B | Group C | Group D |
|---|---|---|---|---|
| 1.0 | 1842.0 | 1942.0 | 1131.0 | 101.0 |
| 2.0 | 1536.0 | 1434.0 | 1756.0 | 112.0 |
| 3.0 | 1756.0 | 1435.0 | 1867.0 | 97.0 |
| 4.0 | 1752.0 | 1453.0 | 1886.0 | 23.0 |
| 5.0 | 1557.0 | 1243.0 | 1947.0 | 26.0 |
| 6.0 | 1584.0 | 1216.0 | 1976.0 | 12.0 |
| 7.0 | 1875.0 | 1216.0 | 1768.0 | 77.0 |
| 8.0 | 1675.0 | 1532.0 | 1467.0 | 78.0 |
| 9.0 | 1764.0 | 1215.0 | 1576.0 | 75.0 |
| 10.0 | 1747.0 | 1215.0 | 1754.0 | 87.0 |
| Average | 1708.8 | 1390.1 | 1712.8 | 68.8 |
| STDEV | 117.2 | 230.0 | 258.7 | 35.6 |

### Example 2: Exemplary Combination Immunotherapy Protocol #2

Female C57/BL6 mice were inoculated with 500,000 B16 melanoma cells intradermally. Cells were purchased from ATCC and grown in complete DMEM media.

After 5 days of tumor growth mice were administered: a) Poly IC (antigen nonspecific immune stimulant 100 ng/mouse, days 5, 6 and 7 after tumor inoculation) (labeled Control); b) Poly IC plus B16 melanoma lysate (sonicated lysate of 1 million cells per mouse, at 5 days after tumor inoculation) (labelled Group B); c) Ozonized syngeneic blood (500 microliters of blood treated with 5 micrograms of ozone per ml) on days 5, 6 and 7 after tumor inoculation) (labelled Group C) and d) Poly IC plus B16 melanoma lysate (sonicated lysate of 1 million cells per mouse, at 5 days after tumor inoculation plus ozone) (Labelled Group D).

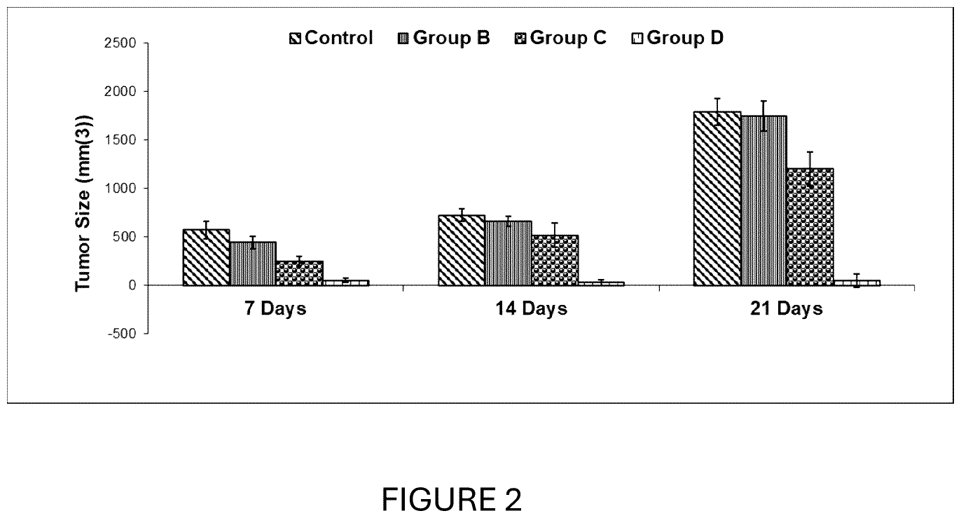

The below Table 4, Table 5, and Table 6 present results at day 14, 21, and 28 respectively, which are also illustrated in FIG. 2.

TABLE 4
Day 14

| | Group A | Group B | Group C | Group D |
|---|---|---|---|---|
| 1.0 | 536.0 | 434.0 | 243.0 | 77.0 |
| 2.0 | 645.0 | 443.0 | 223.0 | 46.0 |
| 3.0 | 745.0 | 453.0 | 254.0 | 76.0 |
| 4.0 | 643.0 | 543.0 | 216.0 | 76.0 |
| 5.0 | 553.0 | 436.0 | 234.0 | 75.0 |
| 6.0 | 563.0 | 475.0 | 243.0 | 53.0 |
| 7.0 | 453.0 | 424.0 | 326.0 | 23.0 |
| 8.0 | 576.0 | 465.0 | 326.0 | 56.0 |
| 9.0 | 432.0 | 426.0 | 324.0 | 43.0 |
| 10.0 | 573.0 | 346.0 | 123.0 | 23.0 |
| Average | 571.9 | 444.5 | 251.2 | 54.8 |
| STDEV | 91.8 | 49.2 | 62.7 | 21.2 |

TABLE 5
Day 21

| | Group A | Group B | Group C | Group D |
|---|---|---|---|---|
| 1.0 | 767.0 | 674.0 | 453.0 | 12.0 |
| 2.0 | 787.0 | 676.0 | 556.0 | 12.0 |
| 3.0 | 746.0 | 354.0 | 545.0 | 115.0 |
| 4.0 | 634.0 | 756.0 | 536.0 | 22.0 |
| 5.0 | 765.0 | 565.0 | 547.0 | 24.0 |
| 6.0 | 635.0 | 746.0 | 435.0 | 21.0 |
| 7.0 | 776.0 | 654.0 | 575.0 | 25.0 |
| 8.0 | 754.0 | 635.0 | 453.0 | 23.0 |
| 9.0 | 632.0 | 776.0 | 563.0 | 25.0 |
| 10.0 | 764.0 | 746.0 | 537.0 | 43.0 |
| Average | 726.0 | 658.2 | 520.0 | 32.2 |
| STDEV | 64.7 | 125.3 | 51.9 | 30.3 |

TABLE 6
Day 28

| | Group A | Group B | Group C | Group D |
|---|---|---|---|---|
| 1.0 | 1746.0 | 1547.0 | 1212.0 | 112.0 |
| 2.0 | 1875.0 | 1767.0 | 1114.0 | 132.0 |
| 3.0 | 1546.0 | 1589.0 | 1008.0 | 198.0 |
| 4.0 | 1775.0 | 1865.0 | 1213.0 | 9.0 |
| 5.0 | 1877.0 | 1578.0 | 1125.0 | 6.0 |
| 6.0 | 1967.0 | 1587.0 | 1242.0 | 9.0 |
| 7.0 | 1565.0 | 1875.0 | 1346.0 | 12.0 |
| 8.0 | 1787.0 | 1657.0 | 1008.0 | 14.0 |
| 9.0 | 1875.0 | 1985.0 | 1535.0 | 12.0 |
| 10.0 | 1887.0 | 2007.0 | 1214.0 | 14.0 |
| Average | 1790.0 | 1745.7 | 1201.7 | 51.8 |
| STDEV | 139.5 | 177.1 | 157.2 | 69.3 |

### Other Considerations

The foregoing description of various embodiments known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y or Z,” unless specifically stated otherwise, is otherwise understood with the context as used in general to present that an item, term, etc., may be either X, Y or Z, or any combination thereof (such as X, Y and/or Z). Thus, such disjunctive language is not generally intended to, and should not, imply that certain embodiments require at least one of X, at least one of Y or at least one of Z to each be present.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (such as a measurement value) is close to a targeted value, where close can mean, for example, the result is within 80% of the value, within 90% of the value, within 95% of the value, or within 99% of the value.

Unless otherwise explicitly stated, articles such as “a” or “an” should generally be interpreted to include one or more described items.

While the above detailed description has shown, described, and pointed out novel features as applied to illustrative embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

The scope of the present disclosure is not intended to be limited by the specific disclosures of examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method of treating cancer comprising the steps of:
a) assessing a cancer patient for sensitivity of the cancer to a variety of therapeutic approaches;
b) reducing tumor hypoxia and/or tumor acidosis in the cancer;
c) altering a tumor microenvironment of the cancer; and
d) administering said cancer therapeutic approaches.

2. The method of claim 1, wherein the method further comprises administering an antigen-specific immune stimulatory means or an antigen-nonspecific stimulatory means.

3. The method of claim 1, wherein assessing a cancer patient for sensitivity to therapeutic approaches comprises assessing cancer samples ex vivo for sensitivity to chemotherapy.

4. The method of claim 1, wherein reducing tumor hypoxia and/or tumor acidosis comprises administering sodium bicarbonate, ozone therapy or hydrogen peroxide.

5. The method of claim 1, wherein altering the tumor microenvironment comprises inducing localized hyperthermia, administering localized high intensity ultrasound, administering localized radiation therapy, localized administration of an immunogenic substance, localized administration of a vaccine adjuvant, localized administration of a vaccine adjuvant, administering low dose chemotherapy, or administering immunogenic chemotherapy.

6. The method of claim 5, wherein the vaccine adjuvant is selected from the group consisting of: Poly IC, incomplete Freunds Adjuvant, and BCG.

7. The method of claim 2, wherein the antigen-specific immune stimulatory means comprises autologous tumor immunization.

8. The method of claim 7, wherein said autologous tumor immunization comprises administration of tumor lysate, tumor exosome, tumor lysate pulsed dendritic cells, tumor exosomes pulsed dendritic cells, or allogeneic tumor cell lines.

9. The method of claim 7, wherein the allogeneic tumor cell lines are made immunogenic by transfection with one or more immunogenic genes.

10. The method of claim 9, wherein said immunogenic genes are selected from the group consisting of: CD40, CD80, CD86, and GM-CSF.

11. The method of claim 3, wherein assessing sensitivity to chemotherapy comprises assessing cancer cell death.

12. The method of claim 11, wherein cancer cell death is assessed by mitochondrial depolarization or by increased membrane permeability to molecules which normally reside outside of said membrane.

13. The method of claim 11, wherein said cancer cell death comprises apoptosis, necrosis, or immunogenic cell death.

14. The method of claim 13, wherein said immunogenic cell death comprises ferroptosis, pyroptosis, or necroptosis.

15. The method of claim 1, wherein assessing the cancer patient for sensitivity to therapeutic approaches comprises assessing cancer samples ex vivo for sensitivity to immunotherapy, metabolic therapy, nucleic acid therapy, electromagnetic therapy, radiation therapy, cryotherapy, hyperthermia, or treatment.

16. A method of predicting a cancer patient's response to therapy comprising the steps of: a) obtaining a tumor sample from said cancer patient; b) exposing said tumor samples to a matrix replicating conditions found inside said tumor microenvironment; c) exposing said tumor sample in said conditions replicating said cancer microenvironment to various cancer treatments being contemplated for us in said cancer patients; d) observing effects of said treatments on said cancer cells; and e) recommending to caregiver of said patient which treatments have evoked an in vitro response.

17. The method of claim 16, wherein the tumor sample is obtained as circulating tumor cells from peripheral blood, and wherein the circulating tumor cells are selected based on expression of PECAM, CD133, CD34, stem cell factor receptor, c-met, TGF-beta receptor, interleukin-1 receptor, interleukin-17 receptor, mucin-1, LIF receptor, IL-10 receptor, based on ability to export rhodamine 231, or based on enhanced binding to lectins.

18. The method of claim 16, wherein the tumor sample comprises a tumor-derived microvesicle, wherein the tumor derived microvesicles are concentrated and exposed to a fibroblast or fibroblast like-population.

19. The method of claim 18, wherein the fibroblast or fibroblast like-population is transfected with one or more oncogenes.

20. The method of claim 16, wherein the tumor sample comprises cancer cells and wherein the method comprises growing cancer cells under conditions of stem cell supernatant.
